(19) United States
(12) Patent Application Publication
(10) Pub. No.: US 2024/0150270 A1
(43) Pub. Date: May 9, 2024

(54) METHOD FOR SELECTIVE RECOVERY OF LIPOPHILIC COMPOUNDS

(71) Applicant: GROW GROUP PLC, London (GB)

(72) Inventors: Christopher James Cordier, London (GB); Sadaf Saad Anjum, London (GB); Benjamin Thomas Langley, London (GB); Ian Joseph Atkinson, London (GB)

(73) Assignee: GROW GROUP PLC, London (GB)

(21) Appl. No.: 18/507,409

(22) Filed: Nov. 13, 2023

Related U.S. Application Data
(63) Continuation of application No. PCT/GB2021/051156, filed on May 13, 2021.

(51) Int. Cl. C07C 37/82; A61K 31/00; A61K 31/05; A61K 36/185; B01D 15/20; B01D 15/32

(52) U.S. Cl. CPC C07C 37/82; A61K 31/05; A61K 31/658; A61K 36/185; B01D 15/203; B01D 15/327; A61K 2236/35; A61K 2236/53

(57) ABSTRACT

A method of selectively recovering a lipophilic target substance. An insoluble adsorbent is exposed to a solution of the target substance in a lipophilic solvent. Adsorption of the adsorbent with the target substance is facilitated by addition of hydrophilic solvent, which is less hydrophobic than the lipophilic solvent, to the solution during or after exposure of the solution to the insoluble adsorbent, lowering the temperature of the solution or evaporating a portion of the lipophilic solvent. The adsorbent Is isolated from the solution. For desorbing and recovering the target substance, a hydrophobic dissociation fluid may be combined with the adsorbent, a temperature of the adsorbent may be increased or a portion of the hydrophilic solvent may be evaporated. A solution may be exposed to one or more adsorbents for recovering additional lipophilic target substances, recovering hydrophilic target substances or removing unwanted substances.

### FIELD

The present disclosure relates to selective recovery of lipophilic compounds.

### BACKGROUND

A significant portion of all drugs prescribed worldwide are natural products sourced from plants. Of 252 drugs described as essential by the World Health Organisation, 11% are exclusively from plant origin and a significant number of the rest are synthetic compounds prepared from naturally occurring precursors. Herbal medicines are treated differently in medicine, pharmacology, and regulatory guidelines than drugs based on isolation and administration of purified active pharmaceutical ingredients (“API”) from plant sources. Clinical trials involving medicines derived from a single API provide health care practitioners with the confidence to prescribe drugs for which there is evidence of efficacy, safety, and reproducible results. The comparative dearth of scientific verification of many herbal medicines may, in the worst-case scenario, be harmful to health, potentially through complications with existing medicines. These concerns may curtail the potential of using whole plant material, or crude extracts, as medicines and hence constrict the therapeutic potential of pharmacological interventions using herbal medicines, despite certain herbal treatments undergoing some degree of clinical certification for efficacy.

One hurdle in bridging the divide between whole plant medicines and contemporary pharmacological standards is the identification of active chemical components of the plant. Extraction and purification of bioactive substances from plant matter has been an active area of investigation since before the introduction of current pharmacological standards and remains a crucial tool in the pharmacologist toolkit today. This approach has allowed the introduction of many widely prescribed essential medicines that are based on a single chemical component of plant origin. There are instances, however, in which no single active chemical component can be identified for a given herbal medicine, even though evidence exists demonstrating the crude whole plant extract is effective in treating a specific disease state. In such cases, thorough analysis of plant extract fractions has shown that more than one active ingredient is required to elicit the desired pharmacological response either in vitro or in vivo.

Cannabis-based medicines are an example of synergy in treatments involving plant derived bioactive substances. Cannabis sativa produces over 140 structurally distinct phytocannabinoids comprising approximately a dozen subclasses based on structural similarity, in addition to a variety of flavanones, terpenoids and other minor constituents that are known to act in concert. This synergistic response to multiple bioactive substances is commonly referred to as the ‘entourage effect’ and is absent when a single phytocannabinoid is administered alone. Administration of a single API such as delta-9-tetrahydrocannabinol (“THC”) can in certain indications lead to an unsatisfying therapeutic outcome with adverse effects including inebriation and short-term memory impairment. As such, deployment of THC in combination with another phytocannabinoid, cannabidiol (“CBD”), has been shown to improve the therapeutic outcome for some conditions and to ameliorate some unwanted side effects.

While the assumption that a single active chemical compound should be responsible for a given therapeutic response has been substantially overturned, the complexity of synergies possible when administering plant-based medicines is enormous when such a large variety of chemical constituents are biosynthesized as by C. sativa. To capitalize on medicinal benefits of this synergy, two approaches have arisen toward broadening the application of cannabis-derived medicines: reconstituting a mixture of active components using purified samples of each constituent, and accurately measuring the content of all bioactive ingredients in a whole plant cannabis extract.

As alluded to above, one ab initio tactic involves attempting to recreate a mixture of APIs in relative quantities that either replicate their natural abundance or can be modified at will to improve outcome or patient experience. A clear advantage of this method is that the therapeutic response can be definitively attributed to the phytocannabinoids added to the reconstituted mixture. As such, regulatory compliance can be navigated through straightforward adaption of current single-API approaches. However, current barriers to full implementation of this approach include: access to all phytocannabinoids in highly purified form is practically very challenging; limited understanding of the relevance of minor bioactive constituents often present in very low quantities below easily detectable levels; high commercial costs of available phytocannabinoids; and the vast range of permutations that exist when attempting to combine these ingredients to simulate the synergistic effect. Future developments that will enable this goal to be realized will include the development of highly efficient extraction technologies that provide the medical and pharmacological communities with cost-effective analytically pure samples of all phytocannabinoids, flavanones, phenylpropanoids and terpenoids.

The alternative approach involves utilizing the broad range of bioactive constituents present in cannabis extracts accessed using current extraction technologies. The effects of administered medicines can be attributed to the unique chemical fingerprint of the extract. This strategy has the benefit that many minor phytocannabinoids can be administered that cannot be easily accessed in sufficient quantities using conventional extraction methods. Conversely, administration of such a broad range of potentially bioactive ingredients presents a complicated challenge that regulatory authorities need to navigate to ensure safe and effective medicines are introduced to the market. Crucial barriers to implementation of this strategy include a lack of authentic samples for analytical confirmation of minor phytocannabinoid content, batch-to-batch variability of metabolite quantities based on plant genetics, growing conditions, harvesting times and extraction method, and inability to detect and accurately measure the levels of minor components that can have either a beneficial influence on synergistic behaviour or a deleterious effect on therapeutic outcome.

In many cases, the regulatory requirements pertaining to analysis of whole plant extracts of C. sativa focus on inebriating phytocannabinoids such as THC, and analysis laboratories do not always possess suitable authentic samples for complete phytocannabinoid profiling. The expense associated with full metabolite profiling leads to wide variability of many minor phytocannabinoids and is often overlooked entirely and results in low patient confidence and low physician confidence in medical utility. As with the ab initio approach to synergistic medical administrations, key technological improvements that will improve the whole plant extract (“WPE”) strategy include the introduction of analytical standard samples or analytical standards of all phytocannabinoids in highly purified form such that in-depth analysis of whole plant extracts can be accurate, low-cost, and routine.

Natural product extraction from botanic sources remains a vibrant area of investigation for pharmacological, pharmaceutical, and medicinal researchers. Natural products chemical architectures provide researchers with a wealth of privileged scaffolds for use in drug discovery, starting points for chemical diversification toward novel libraries, and inspiration for de novo synthesis of natural product-like libraries. To continue and expand the benefit of natural products in the development of new medicines, new approaches to the extraction, isolation, and purification of naturally occurring small molecules is crucial and often requires techniques, protocols, and strategies to be tailored to the target compound of interest.

Previous approaches to extraction of phytocannabinoids and other metabolites found in C. sativa have involved the use of conventional organic solvents, supercritical fluid, butane and related volatile organic media. Deep eutectic solvents and ionic liquids have been used for the extraction of plant metabolites from plant matter, including from C. sativa. Each of the above approaches provide an extraction technique that can be somewhat tailored to the isolation of certain plant metabolite subclasses from other natural product classes but also presents unique challenges. Selectivity for phytocannabinoids of interest, isolation or removal of fatty acids and waxes, infrastructural requirements, pre-extraction requirements such as drying and wax winterization procedures, or post-extraction treatments such as solvent removal operations all require bespoke solutions.

A common challenge to most conventional extraction methods is the necessity to dry plant material prior to extraction using a non-aqueous solvent. Drying is a hugely costly process since it requires reducing water content from the harvested plant, which can be 70-90% humidity, to closer to 5-15% water content. Disruptive new technology will use innovative solutions that bypass this energy and cost-intensive impediment. One strategy that circumvents the necessity for arduous drying protocols, is water-steam distillations, that have found some utility in the extraction of plant metabolites in some specific applications. For example, isolation of essentials oils and terpenoids have benefited from steam distillation techniques but such approaches have not been widely functional for more structurally complex or chemically sensitive compounds that are sensitive to heat or suffer from bespoke challenges associated with physicochemical tolerance to water environments.

Ethanol extracts of cannabis have the benefit of using a sustainable, renewable solvent that is generally regarded as safe even if trace quantities are present when consumed. This solvent can be effective at removing many plant metabolites from the whole plant biomass but comes at the cost of requiring multiple processing operations downstream of the initial extraction. For an organic solvent, ethanol is highly polar and as such removes not only the highly lipophilic components such as phytocannabinoids extracted from cannabis but also unwanted water-soluble compounds such as chlorophyll. Post extraction processing is generally more extensive and labour intensive than extractions using alternative protocols and solvents. Ethanol has a notably higher boiling point compared with shorter hydrocarbons such as butane or propane and this property makes solvent removal slower, more difficult, and costly.

Supercritical fluid technology, particularly using carbon dioxide, is an area of continuing improvement within the field of natural product extraction from plant material. This method of extraction has been particularly well utilized within the cannabis industry for the isolation of phytocannabinoids, flavonoids, phenylpropanoids, terpenoids, and other constituents of value for patients and consumers. Supercritical carbon dioxide extraction systems have the advantage of being non-flammable, and can be performed at temperatures that can be attenuated for the isolation of temperature-sensitive plant metabolites. A major disadvantage of supercritical fluid extraction is the high barrier to entry by means of investment in costly infrastructure requirements and the maintenance of said instruments by technically skilled personnel. The use of such a setup does not negate any of the pre- and post-extraction operations mentioned above for ethanol extraction. Supercritical fluid extraction functions as a wide-net capture method without providing highly selective operating conditions for the isolation of specific plant metabolites. In addition, while the use of supercritical methods may be described as a solvent-free extraction approach, a solvent is frequently required during post-extraction operations when unwanted extracted compounds are removed in order to prepare pharmaceutical-grade active components.

Isolation of plant metabolites using highly lipophilic solvents such as hydrocarbons presents an attractive option when target molecules demonstrate non-optimal solubility in more polar media. A major disadvantage with this technology is the use of highly flammable material that represents an explosion risk. In addition, hydrocarbon-based extracts are more likely to contain trace solvent residues. In applications to the cannabis industry, this drawback can lead to flavour anomalies and potentially as lung irritation.

Deep eutectic solvents (“DES”), natural deep eutectic solvents (“NADES”), and ionic liquids (“L”) have been used for the extraction of plant metabolites from plant material. Within this context, such solvents have been used in the extraction of phytocannabinoids from C. sativa plant matter. These solvents are non-trivial to remove and as such isolation of purified samples of phytocannabinoids from these solvents post-extraction from the plant matter presents a barrier to their application.

There exists a number of peer-reviewed literature articles pertaining to the application of deep eutectic solvents and ionic liquids for the extraction of plant metabolites, including phytocannabinoids and specific applications for the isolation of broader classes of phenolic compounds from various plant sources.

In one approach to circumvent solvent removal challenges when performing extractions using DESs, NADESs, or ILs, such solvent mixtures have been used in conjunction with macroporous polymeric capturing devices to remove extracted natural products from these non-volatile solvents. Macroporous resins have been used in extraction protocols more generally, including the isolation of flavonoids from plant material following supercritical fluid extraction.

### SUMMARY

Plant-derived medicinal products are crucial tools to complement pharmacological treatments utilising single component APIs, as well as being a source and starting point for new APIs. Treatment using herbal extracts often benefit from synergistic behaviours of multiple active species produced by the plant but suffer from limitations regarding formulations, dosing irregularities, and chemovar variabilities. Comprehensive chemical analysis of whole plant extracts is often expensive and complicated by a lack of accuracy in determining levels of minor constituents that may be relevant to the overall therapeutic outcome. Access to standard analytical samples of minor plant constituents may greatly improve the regulatory oversight for these treatments and provide medical practitioners with greater confidence when prescribing whole plant medicines. Efforts to isolate each bioactive component with a view to consistently providing an accurately prepared mixture of active ingredients may overcome extract irregularities but demands commercial access to all active species in highly purified form in order to reconstitute that which is observed in the crude plant extract.

Contemporary approaches to extraction of plant metabolites, such as phytocannabinoids, have involved the use of conventional alcohols, organic solvents, supercritical fluids, hydrocarbons and related volatile organic media. Less conventional approaches include employing deep eutectic solvents and ionic liquids for extraction of plant metabolites from plant biomass and have also been applied to phytocannabinoid isolation. The above approaches do not provide a highly tunable approach to the separation of various plant metabolites subclasses from unwanted natural product classes, contaminants and impurities, or for the isolation of specific compounds from said mixtures. Non-specific extractions of this kind can capture a wide variety of compound classes from the plant, such as fatty acids, waxes, and chlorophyll; crucially, these techniques do not benefit from chemical structure-based specificity. Often the strategies used in extractions require high infrastructure costs, highly flammable solvents, and expensive pre- and post-extraction treatments. Such approaches may ultimately provide insufficient quantities of minor phytocannabinoids at highly inflated prices. The pharmacological understanding and expansion of plant-based medicines requires new extraction technologies that can be tailored to specific metabolites of interest that are easy to operate, provides bioactive compounds in high purity, increase efficiency and substantially reduces costs.

In view of the shortcomings in extraction technology, there is motivation to produce an approach to capturing plant metabolites, such as polyphenolics, phytocannabinoids, terpenoids, or other plant metabolites that utilizes and expands upon the known guest-host molecular interactions governing encapsulation into adsorbents.

The method provided herein provides solid phase adsorption of hydrophobic target compounds from liquid phase solutions by application of one or more adsorbents. Adsorbents have guest-host molecular interaction that are defined by a chemical structure, macromolecular structure, supramolecular structure, nanostructure, or microstructure that imparts selective or specific affinities for individual components of a mixed composition. Where two or more adsorbents are applied, one of the adsorbents may be directed to adsorption of other lipophilic target compounds or to adsorption of hydrophilic target compounds.

Adsorbents may display selectivity for classes of compounds, or specificity for individual compounds, according to, for example, surface chemistry, pore size, cavity dimensions, stereoelectronic environment, or complimentary polarity of the adsorbent material matrix, and may be selected based on these features, or other structural and physicochemical properties, with or without further surface or chemical modification, to bind a target of interest or range of targets.

Some examples of adsorbents that may be applied to adsorption of lipophilic compounds include cyclic polysaccharides, which may include cyclodextrins, which may include α-cyclodextrin, β-cyclodextrin or γ-cyclodextrin. Additional examples of adsorbents that may be applied to adsorption of lipophilic compounds include silica gel, cyclodextrin-IPI, cyclodextrin-MPI, PTFE Granules, oligosaccharides, non-cyclic polysaccharides, amylose-HDI, Merrifield PVB/DVB resin, cyclodextrin-TDI, maltodextrin-HDI, cyclodextrin-HDI, and cyclodextrin-HDI with brine. Additional examples of adsorbents that may be applied to adsorption of lipophilic compounds include biopolymers, with or without synthetic modification, such as branched and linear polysaccharides, oligosaccharides, acetylated cellulose, peptides, proteins, polymerized adducts of amino acids (e.g. melanin, etc.), polyphenolic scaffolds (e.g. lignin, suberin, etc.), polymeric isoprenes (e.g. rubber, etc.), and fatty acid polyesters (cutin, cutan, etc.), including after grafting, cross-linking, blending or coating to impart selected solubility properties. Additional examples of adsorbents that may be applied to adsorption of lipophilic compounds include alumina, zeolitic molecular sieves and silicon dioxide.

Some examples of adsorbents that may be applied to adsorption of hydrophilic compounds include sand, Amberlite® XAD-4 neutral resin, vermiculite, cellulosic fibres, silicon-coated cellulosic fibres, Fuller's earth, nanoclay hydrophilic bentonite, clay mineral blend, wood pulp, 3 Å molecular sieves, Celite®, and Dowex® 1×8 strongly basic resin. Some examples of porous adsorbents that may be applied to adsorption of hydrophilic compounds include silica gel, diatomaceous earth, bleaching earth, activated clays, activated carbons and charcoals, magnesium oxide, alumina, activated alumina, zeolitic molecular sieves, bauxites and silicon dioxide. Some examples of non-porous adsorbents that may be applied to adsorption of hydrophilic compounds include sodium sulphate and magnesium sulphate. Some examples of adsorbents that may be applied to adsorption of hydrophilic compounds include branched and linear polysaccharides, oligosaccharides, peptides and proteins.

Polysaccharide mixtures have been used to improve the water solubility of phytocannabinoid and whole plant extracts of cannabis, allowing greater control over dosing and formulation. Applications of cyclic polysaccharides, such as cyclodextrins, have shown structure-dependent guest-host molecular interactions between the sugar (host) and the phytocannabinoid (guest) in resulting inclusion complexes, conceptually similar to a lock and key. Silica-bound cyclodextrins and derivatives, have been utilized previously for their ability to selectively bind small molecules in a structure-dependent manner when constructed into chromatography columns but have not been optimized for phytocannabinoid extraction applications. In addition, polymeric cyclic polysaccharides, such as cyclodextrins, have been used to remove phenolic compounds during water purification, including with selectivity for certain phenols based on chemical structures but have not been utilized as a tool for plant metabolite extraction. Cyclodextrins contained within polymeric matrices used for phenol decontamination from water have not been utilized in the extraction of plant metabolites from plant matter.

The method provided herein applies structure-specific guest-host molecular interactions between lipophilic compounds, such as phytocannabinoids or other plant metabolites, and the polysaccharide. Cyclodextrins, derivatives, or similar cyclic polysaccharides are applied as a polymeric framework that permits capture, release, and hence purification of plant metabolites from a solution of whole plant extract. In the method disclosed herein, cyclodextrins, including α-cyclodextrin, β-cyclodextrin and γ-cyclodextrins, or similar cyclic polysaccharides, may be incorporated into polymeric frameworks that permit facile separation of bound target compounds from unwanted plant debris or undesired plant metabolites. Different cavity sizes may be applied for the capture of plant metabolites based on guest-host compatibility due to molecular size and shape.

Distinct insoluble adsorbents may be applied simultaneously or sequentially during exposure to an extractant solution or other mixture including a lipophilic target compound. Adsorbents may demonstrate non-selective binding modest affinity, or high target specificity for certain plant metabolites. In other cases, one adsorbent may demonstrate strong coordination to contaminants such as metal salts, while a different adsorbent may strongly bind to unwanted coloured impurities such as pigments and chlorophyllic compounds, while another adsorbent may display effective adhesion to all lipophilic compounds. By combining different adsorbents serially or simultaneously, defined binding patterns and purification pathways may be applied.

The adsorbent may be embedded onto a chromatography medium or other insoluble matrix, which may be used in a slurry, coated to a surface such as silica gel, embedded within a chromatography device or otherwise applied to selective recovery of lipophilic compounds. The chromatography medium may be applied in a chromatography column for use with instrument including high-pressure liquid chromatography, supercritical fluid chromatography or manual chromatography applications. This chromatography medium may be used in direct substitution with conventional silica gel or contained within chromatography devices such as a chromatography column for use with instrument including high-performance liquid chromatography (“HPLC”), supercritical fluid chromatography, manual chromatography applications or related approaches, for chromatographic separation of plant metabolites from unwanted plant material or metabolites, or from the solvent mixture itself. Elution using solvents that perturb the binding affinity of the target molecule to the capture device allowing release of target molecules in highly purified or enriched form. Where chromatography is applied with cyclic polysaccharides, the insoluble cyclic polysaccharides may be provided with at least two cyclic polysaccharide molecules for each subunit that is attached to the immobile phase. Single or greater numbers of molecules for each subunit at each attachment point to the immobile phase may be applied for other adsorbents.

Adsorbents may be bound, such as by covalent attachment, to a fibrous chromatography matrix such as glass fibre, cellulosic material or alternative matrices. Fibrous material of this kind can be used to pack chromatography columns, or used as a polymer inclusion membrane or alternatives for recovery of lipophilic target substances from whole plant extracts derived from conventional organic solvents, water, deep eutectic solvents, ionic liquids or a mixture thereof, following filtration of plant debris, with elution of solution-phase unwanted plant metabolites. Upon sequestering of the adsorbents bound to the lipophilic target substances from the unwanted plant metabolites that remain in solution, elution from the matrix may be promoted by addition of lipophilic solvents, such as conventional organic solvents, aqueous mixtures, deep eutectic solvents, natural deep eutectic solvents or ionic liquids, or the application of heat, to disrupt the guest-host environment and promote release of metabolites of interest into solution in purified or enriched quantities.

Insoluble adsorbents may be contained within a water-permeable immersion filter with a mesh size smaller than the adsorbent particle size, or groups of adsorbents may be housed together within a plurality of immersion filters. The adsorbents may be covalently bound with the permeable mesh of the immersion filter or simply sequestered inside by having an insoluble adsorbent with a particle size greater than the mesh size of the permeable material. Immersion filters may be distinguished from each other by suitable means, for example by tethering to a fixed location, barcoding, or incorporation of a unique radiofrequency identification (RFID) tag. Multiple immersion filters may be contacted with an extractant solution throughout the entire process, or may be added and withdrawn at different points according to a selected order for removing different target compounds.

Where solvent switches, or renewal of adsorbents is applied to a system with multiple immersion filters, an adsorbent having bound a contaminant, impurity or other secondary target compound may be removed from the solution and replaced with an immersion filter having the same adsorbent or different adsorbents.

Target compounds may be adsorbed onto, or dissociated from, the adsorbents by modification of solvated/liquid conditions to provide a driving force for target adsorption onto fresh adsorbent, withdrawal, drying, and elution, providing purified target compounds. Driving forces may include solvent evaporation to dryness to provide a solventless residue that may be redissolved in a different solvent. Similar solvent transitions can be achieved by first addition of excess adsorbent to the solution, whether housed within an immersion filter or as a free-flowing solid, facilitating binding of the target compound to the adsorbent. Subsequently, addition of excess insolubilizing solvent, addition of solutes (e.g. alkali metal halide salts, alkaline metal halide salts, alkali nitrates, alkaline metal nitrates, alkali sulphates, alkaline metal sulphates and alkali phosphates, alkaline metal phosphates, NaCl, KCl, MgCl₂, CaCl₂, Na₂SO₄, K₂SO₄, MgSO₄, CaSO₄, etc.), lowering temperatures to decrease solubility or evaporating lipophilic solvent to decrease solubility may further drive the lipophilic target compounds onto the adsorbent. Addition of solubilising solvents to the adsorbent, raising temperatures, or evaporating insolubilizing solvent, facilitates release of adsorbed target compounds and removal of adsorbent sets the stage for subsequent repetitions of the above cycles.

Following attenuation of liquid phase conditions to lower solubility as described above, a particular compound may approach the saturation point and bind onto the surface of the adsorbent to which greatest affinity exists, as dictated by the structural and chemical properties of both small molecule guest and adsorbent host. Where immersion filters are applied, some or all of the immersion filters, selected based on which adsorbent is on which filter, may be removed, replaced or substituted with new immersion filters containing the same or different adsorbents. The removed immersion filters may be dissociated in a dissociation solvent, exposed to high temperature to facilitate dissociation or otherwise exposed to conditions that change solubility.

For storage of active ingredients, target bound adsorbents may be dried to a free-flowing powder for ease of handling and purged with an inert gas to prevent aerobic degradation. Post-desorption, adsorbent recycling may involve excessive rinsing with varied solvent mixtures or surfactants that may disrupt surface interactions to remove trace materials. After use, adsorbents may be recovered and used again.

Adsorbents may be applied as an insoluble polymeric material, ground to a fine powder, milled into beads, appended to magnetic nanoparticles or insoluble magnetic beads. Such polymers may be added to crude extracts of plant material derived from conventional organic solvents, water, deep eutectic solvents, ionic liquids, or a mixture thereof, following filtration of plant debris. Attenuation of the solvent mixture promotes capture of compounds of interest, and filtration or other recovery of the insoluble polysaccharide permits physical exclusion of target metabolites from the solvent. Suspension of metabolite-bound polymers in a dissociation solvent or other dissociation fluid, application of heat or other changes in the solvent mixture promotes release of captured lipophilic target compounds as purified compounds.

Solvents that interrupt the non-covalent binding interactions between lipophilic target substances and a given adsorbent host may be applied to recover the lipophilic target substances. When these interactions are interfered with, the binding affinity of the lipophilic target substance with the adsorbent is substantially reduced and the encapsulation process is overturned, releasing target compounds into solution, or in vapour form if applying heat, thereby allowing recovery of the lipophilic target substance. Such dissociation fluids may include conventional organic solvents, supercritical fluids, water, deep eutectic solvents, ionic liquids, or a mixture thereof.

Upon release of target molecules bound to adsorbent, a cleaning solvent may be used to strip away any compounds still bound to the polymeric matrix. At this stage, the adsorbent offers the same binding motifs that were present prior to any capturing and as such can be reused multiple times using a sequence of capture, release, and cleaning, based on the solvent applied.

In a first aspect, herein provided a method of selectively recovering a lipophilic target substance. An insoluble adsorbent is exposed to a solution of the target substance in a lipophilic solvent. Adsorption of the adsorbent with the target substance is facilitated by addition of hydrophilic solvent, which is less hydrophobic than the lipophilic solvent, to the solution during or after exposure of the solution to the insoluble adsorbent, lowering the temperature of the solution or evaporating a portion of the lipophilic solvent. The adsorbent is isolated from the solution. For desorbing and recovering the target substance, a hydrophobic dissociation fluid may be combined with the adsorbent, a temperature of the adsorbent may be increased or a portion of the hydrophilic solvent may be evaporated. A solution may be exposed to one or more adsorbents for recovering additional lipophilic target substances, recovering hydrophilic target substances or removing unwanted substances.

In a further aspect, herein provided is a method of selectively recovering a lipophilic target substance, the method comprising: providing a solution comprising the target substance in a lipophilic solvent; combining an adsorbent with the solution; combining an adsorption solvent with the solution, the adsorption solvent being less hydrophobic than the lipophilic solvent to facilitate binding of the adsorbent with the target substance; and isolating the adsorbent from the solution; wherein the adsorbent is insoluble in the solution; and the adsorbent is selected from the group consisting of amylose, maltodextrin, silica gel, polytetrafluoroethylene granules, chloromethylpolystyrene-divinylbenzene resin, polyvinyl butyral resin and acetylated cellulose.

In some embodiments, providing the solution comprises combining bulk plant material with the lipophilic solvent and separating the bulk plant material from the lipophilic solvent. In some embodiments, the bulk plant material comprises material from Cannabis sativa and the target substance comprises a phytocannabinoid. In some embodiments, the lipophilic solvent comprises an organic solvent. In some embodiments, the organic solvent is selected from the group consisting of acetone, acetonitrile, tetrahydrofuran, glycerol, DMSO, dichloromethane and chloroform. In some embodiments, the organic solvent comprises an alcohol. In some embodiments, the alcohol is selected from the group consisting of methanol, ethanol, n-propyl alcohol and isopropyl alcohol. In some embodiments, the organic solvent comprises a hydrocarbon. In some embodiments, the hydrocarbon is selected from the group consisting of n-hexane, butane and propane. In some embodiments, the lipophilic solvent comprises a eutectic solvent. In some embodiments, the eutectic solvent is selected from the group consisting of glucose syrup, and acetic acid mixed with menthol. In some embodiments, the lipophilic solvent comprises an ionic liquid. In some embodiments, the ionic liquid comprises 1-butyl-3-methylimidazolium tetrafluoroborate. In some embodiments, the adsorption solvent comprises water. In some embodiments, the adsorption solvent comprises a chelating agent. In some embodiments, combining the adsorption solvent with the solution comprises evaporating at least a portion of the lipophilic solvent prior to combining the adsorption solvent with the solution. In some embodiments, the adsorbent comprises amylose or maltodextrin and is cross-linked with a cross-linker selected from the group consisting of hexamethylene diisocyanate, isophorone diisocyanate, 4,4′-methylenebis(phenyl isocyanate) and tolylene-2,4-diisocyanate. In some embodiments, the adsorbent comprises beads that are insoluble in the solution and isolating the adsorbent from the solution comprises filtering the beads out of the solution. In some embodiments, the beads comprise a magnetic substance and isolating the adsorbent from the solution comprises magnetically attracting the magnetic substance. In some embodiments, the adsorbent comprises nanoparticles of a magnetic substance and isolating the adsorbent from the solution comprises magnetically attracting the magnetic substance. In some embodiments, the adsorbent comprises a powder that is insoluble in the solution. In some embodiments, the adsorbent comprises a gel matrix that is insoluble in the solution. In some embodiments, the adsorbent is sequestered within a permeable material. In some embodiments, the permeable material comprises an immersion filter; and isolating the adsorbent from the solution comprises removing the immersion filter from the solution. In some embodiments, the immersion filter carries an identifiable feature for identifying the immersion filter. In some embodiments, the identifiable feature is selected from the group consisting of a radio frequency identification signal, a physical tag, a barcode, colour-coding of the immersion filters, and other visual labelling. In some embodiments, the adsorbent is added to the solution before combining the adsorption solvent with the solution. In some embodiments, the adsorbent is added to the solution after combining the adsorption solvent with the solution. In some embodiments, the target substance is ionizable in the solution, and further comprising combining a solute with the solution for competing with the secondary target substance for binding on the secondary adsorbent to dissociate the secondary target substance from the secondary adsorbent into the solution, for recovering the target substance. In some embodiments, the method comprises increasing a temperature of the adsorbent for dissociating the target substance from the adsorbent and recovering the target substance. In some embodiments, the method comprises evaporating at least a portion of the adsorption solvent for dissociating the target substance from the adsorbent and recovering the target substance. In some embodiments, the method comprises combining a dissociation fluid with the adsorbent for dissociating the target substance from the adsorbent and recovering the target substance. In some embodiments, the dissociation fluid comprises a fluid selected from the group consisting of methanol, ethanol, n-propyl alcohol and isopropyl alcohol, other alcohols, acetone, acetonitrile, tetrahydrofuran, glycerol, DMSO, dichloromethane, chloroform, other organic solvents, n-hexane, butane, propane, other hydrocarbons, glucose syrup, acetic acid mixed with menthol, other eutectic solvents, 1-butyl-3-methylimidazolium tetrafluoroborate, other ionic liquids, a heated gas, a pressurized gas, subcritical CO2, other subcritical fluids, supercritical CO2 or other supercritical fluids. In some embodiments, the dissociation fluid has a lower volume than the lipophilic solvent for concentrating the target substance relative to the concentration of the target substance in the solution. In some embodiments, the dissociation fluid is more hydrophobic than the lipophilic solvent. In some embodiments, combining a secondary adsorbent with the solution for binding of the secondary adsorbent with a secondary target substance; combining a secondary adsorption solvent with the solution to facilitate binding of the secondary adsorbent with the secondary target substance; and isolating the secondary adsorbent from the solution; wherein the secondary adsorbent is insoluble in the solution; and the secondary adsorbent carries a structural property or physicochemical property corresponding to a structural property or physiochemical property of the secondary target substance to preferentially adsorb to the secondary target substance over the target substance. In some embodiments, the structural property or physicochemical property is selected from the group consisting of surface chemistry, pore size, cavity dimension, stereoelectronic environment and polarity. In some embodiments, the secondary target substance is a secondary lipophilic target substance, the secondary adsorption solvent is more hydrophilic than the solution and the secondary adsorbent is selected from the group consisting of a cyclic polysaccharide, amylose, maltodextrin, silica gel, polytetrafluoroethylene granules, chloromethylpolystyrene-divinylbenzene resin, polyvinyl butyral resin, acetylated cellulose, branched polysaccharides, linear polysaccharides, oligosaccharides, peptides, proteins, polymerized adducts of amino acids, polyphenolic scaffolds, polymeric isoprenes, fatty acid polyesters, alumina, zeolitic molecular sieves and silicon dioxide. In some embodiments, the secondary adsorbent comprises a cyclic polysaccharide and the cyclic polysaccharide comprises a secondary cyclodextrin. In some embodiments, the secondary cyclodextrin is selected from the group consisting of α-cyclodextrin, β-cyclodextrin and γ-cyclodextrin. In some embodiments, the secondary adsorbent comprises amylose or maltodextrin. In some embodiments, the secondary adsorbent is cross-linked with a cross-linker selected from the group consisting of hexamethylene diisocyanate, isophorone diisocyanate, 4,4′-methylenebis(phenyl isocyanate) and tolylene-2,4-diisocyanate. In some embodiments, the secondary target substance is a hydrophilic target substance, the secondary adsorption solvent is more hydrophobic than the solution and the secondary adsorbent is selected from the group consisting of sand, Amberlite® XAD-4 neutral resin, vermiculite, cellulosic fibres, silicon-coated cellulosic fibres, Fuller's earth, nanoclay hydrophilic bentonite, clay mineral blend, wood pulp, 3 Å molecular sieves, Celite®, Dowex® 1×8 strongly basic resin, silica gel, diatomaceous earth, bleaching earth, activated clays, activated carbons and charcoals, magnesium oxide, alumina, activated alumina, zeolitic molecular sieves, bauxites, silicon dioxide, sodium sulphate, magnesium sulphate, branched and linear polysaccharides, oligosaccharides, peptides and proteins. In some embodiments, the secondary adsorbent comprises beads that are insoluble in the solution and isolating the secondary adsorbent from the solution comprises filtering the beads out of the solution. In some embodiments, the beads comprise a magnetic substance and isolating the secondary adsorbent from the solution comprises magnetically attracting the magnetic substance. In some embodiments, the secondary adsorbent comprises nanoparticles of a magnetic substance and isolating the secondary adsorbent from the solution comprises magnetically attracting the magnetic substance. In some embodiments, the secondary adsorbent comprises a powder that is insoluble in the solution. In some embodiments, the secondary adsorbent comprises a gel matrix that is insoluble in the solution. In some embodiments, the secondary adsorbent is sequestered within a secondary permeable material. In some embodiments, the secondary permeable material comprises a secondary immersion filter; and isolating the secondary adsorbent from the solution comprises removing the secondary immersion filter from the solution. In some embodiments, the secondary immersion filter carries an identifiable feature for identifying the secondary immersion filter. In some embodiments, the identifiable feature is selected from the group consisting of a radio frequency identification signal, a physical tag, a barcode, colour-coding of the immersion filters, and other visual labelling. In some embodiments, combining the adsorbent with the solution takes place sequentially with combining the secondary adsorbent with the solution. In some embodiments, combining the secondary adsorbent with the solution takes place following combining the adsorbent with the solution. In some embodiments, combining the secondary adsorbent with the solution takes place prior to combining the adsorbent with the solution. In some embodiments, combining the adsorbent with the solution takes place simultaneously with combining the secondary adsorbent with the solution. In some embodiments, the secondary target substance is ionizable in the solution, and further comprising combining a solute with the solution for competing with the secondary target substance for binding on the secondary adsorbent to dissociate the secondary target substance from the secondary adsorbent into the solution, for recovering the target substance. In some embodiments, further comprising increasing a temperature of the secondary adsorbent for dissociating the secondary target substance from the secondary adsorbent and recovering the target substance. In some embodiments, the method comprises evaporating at least a portion of the secondary adsorption solvent for dissociating the secondary target substance from the secondary adsorbent and recovering the secondary target substance. In some embodiments, the method comprises combining a secondary dissociation fluid with the secondary adsorbent for recovering the secondary target substance, wherein the secondary target substance is more soluble in the secondary dissociation fluid than in the solution.

In a further aspect, herein provided is a method of selectively recovering a lipophilic target substance comprising: providing a solution comprising the target substance in a lipophilic solvent; combining an adsorption solvent with the solution, the adsorption solvent being less hydrophobic than the lipophilic solvent to facilitate binding of the target substance with an adsorbent; exposing the solution to a chromatography medium, the chromatography medium comprising the adsorbent for binding to the target substance; and exposing a dissociation fluid to the chromatography medium for eluting the target substance in an eluted solution; wherein the adsorbent is bound with the chromatography medium; and the adsorbent is selected from the group consisting of amylose, maltodextrin, silica gel, polytetrafluoroethylene granules, chloromethylpolystyrene-divinylbenzene resin, polyvinyl butyral resin and acetylated cellulose.

In some embodiments, providing the solution comprises combining bulk plant material with the lipophilic solvent and separating the bulk plant material from the lipophilic solvent. In some embodiments, the bulk plant material comprises material from Cannabis sativa and the target substance comprises a phytocannabinoid. In some embodiments, the lipophilic solvent comprises an organic solvent. In some embodiments, the organic solvent is selected from the group consisting of acetone, acetonitrile, tetrahydrofuran, glycerol, DMSO, dichloromethane and chloroform. In some embodiments, the organic solvent comprises an alcohol. In some embodiments, the alcohol is selected from the group consisting of methanol, ethanol, n-propyl alcohol and isopropyl alcohol. In some embodiments, the lipophilic solvent comprises a hydrocarbon. In some embodiments, the hydrocarbon is selected from the group consisting of n-hexane, butane and propane. In some embodiments, the lipophilic solvent comprises a eutectic solvent. In some embodiments, the eutectic solvent is selected from the group consisting of glucose syrup, and acetic acid mixed with menthol. In some embodiments, the lipophilic solvent comprises an ionic liquid. In some embodiments, the ionic liquid comprises 1-butyl-3-methylimidazolium tetrafluoroborate. In some embodiments, the adsorption solvent comprises water. In some embodiments, the adsorption solvent comprises a chelating agent. In some embodiments, the adsorbent is cross-linked with a cross-linker selected from the group consisting of hexamethylene diisocyanate, isophorone diisocyanate, 4,4′-methylenebis(phenyl isocyanate) and tolylene-2,4-diisocyanate. In some embodiments, the adsorbent is covalently bound with the chromatography medium. In some embodiments, the adsorbent is cross-linked with the chromatography medium by a cross-linking agent. In some embodiments, the adsorbent comprises amylose or maltodextrin and the cross-linking agent is selected from the group consisting of hexamethylene diisocyanate, isophorone diisocyanate, 4,4′-methylenebis(phenyl isocyanate) and tolylene-2,4-diisocyanate. In some embodiments, the chromatography medium comprises media selected from the group consisting of cellulose, other carbohydrates and silica. In some embodiments, the dissociation fluid is more hydrophobic than the solution comprising the lipophilic solvent and the adsorption solvent. In some embodiments, the dissociation fluid comprises a fluid selected from the group consisting of methanol, ethanol, n-propyl alcohol and isopropyl alcohol, other alcohols, acetone, acetonitrile, tetrahydrofuran, glycerol, DMSO, dichloromethane, chloroform, other organic solvents, n-hexane, butane, propane, other hydrocarbons, glucose syrup, acetic acid mixed with menthol, other eutectic solvents, 1-butyl-3-methylimidazolium tetrafluoroborate, other ionic liquids, a heated gas, a pressurized gas, subcritical CO2, other subcritical fluids, supercritical CO2 or other supercritical fluids. In some embodiments, the dissociation fluid has a lower volume than the lipophilic solvent for concentrating the target substance relative to the concentration of the target substance in the solution. In some embodiments, the method includes exposing the solution to a secondary chromatography medium, the secondary chromatography medium comprising as secondary adsorbent for binding to as secondary target substance; wherein the secondary adsorbent carries a structural property or physicochemical property corresponding to a structural property or physiochemical property of the secondary target substance to preferentially adsorb to the secondary target substance over the target substance. In some embodiments, the structural property or physicochemical property is selected from the group consisting of surface chemistry, pore size, cavity dimension, stereoelectronic environment and polarity. In some embodiments, the secondary target substance is a secondary lipophilic target substance and the secondary adsorbent is selected from the group consisting of a cyclic polysaccharide, amylose, maltodextrin, silica gel, polytetrafluoroethylene granules, chloromethylpolystyrene-divinylbenzene resin, polyvinyl butyral resin, acetylated cellulose, branched polysaccharides, linear polysaccharides, oligosaccharides, peptides, proteins, polymerized adducts of amino acids, polyphenolic scaffolds, polymeric isoprenes, fatty acid polyesters, alumina, zeolitic molecular sieves and silicon dioxide. In some embodiments, the secondary adsorbent comprises a cyclic polysaccharide and the cyclic polysaccharide comprises a secondary cyclodextrin. In some embodiments, the secondary cyclodextrin is selected from the group consisting of α-cyclodextrin, β-cyclodextrin and γ-cyclodextrin. In some embodiments, the secondary adsorbent comprises amylose or maltodextrin. In some embodiments, the secondary adsorbent is cross-linked with a cross-linker selected from the group consisting of hexamethylene diisocyanate, isophorone diisocyanate, 4,4′-methylenebis(phenyl isocyanate) and tolylene-2,4-diisocyanate. In some embodiments, the secondary target substance is a hydrophilic target substance and the secondary adsorbent is selected from the group consisting of sand, Amberlite® XAD-4 neutral resin, vermiculite, cellulosic fibres, silicon-coated cellulosic fibres, Fuller's earth, nanoclay hydrophilic bentonite, clay mineral blend, wood pulp, 3 Å molecular sieves, Celite®, Dowex® 1×8 strongly basic resin, silica gel, diatomaceous earth, bleaching earth, activated clays, activated carbons and charcoals, magnesium oxide, alumina, activated alumina, zeolitic molecular sieves, bauxites, silicon dioxide, sodium sulphate, magnesium sulphate, branched and linear polysaccharides, oligosaccharides, peptides and proteins. In some embodiments, exposing the solution to the secondary chromatography medium exposing the solution to the chromatography medium. In some embodiments, the method comprises exposing a secondary dissociation fluid with the chromatography medium for eluting the secondary target substance in a secondary eluted solution.

In a further aspect, herein provided is a method of selectively recovering a phytocannabinoid, the method comprising: providing a solution comprising the phytocannabinoid in an organic solvent; combining an adsorbent with the solution, the adsorbent being insoluble in the solution; combining a hydrophilic solvent with the solution to facilitate binding of the adsorbent with the phytocannabinoid; and isolating the adsorbent from the solution; wherein the adsorbent is selected from the group consisting of amylose, maltodextrin, silica gel, polytetrafluoroethylene granules, chloromethylpolystyrene-divinylbenzene resin, polyvinyl butyral resin and acetylated cellulose.

In a further aspect, herein provided is aa method of selectively recovering a lipophilic target substance, the method comprising: providing a solution comprising the target substance in a lipophilic solvent; combining an adsorbent with the solution, the adsorbent being insoluble in the solution; combining a solute with the solution for decreasing the hydrophobicity of the solution to facilitate binding of the adsorbent with the target substance; and isolating the adsorbent bound with the target substance from the solution; wherein the adsorbent is selected from the group consisting of amylose, maltodextrin, silica gel, polytetrafluoroethylene granules, chloromethylpolystyrene-divinylbenzene resin, polyvinyl butyral resin and acetylated cellulose.

In some embodiments, the solute comprises a substance selected from the group consisting of a sugar, a salt, an acid and a base. In some embodiments, the solute comprises a sugar selected from the group consisting of glucose, fructose, galactose, sucrose, lactose and maltose. In some embodiments, the solute comprises a salt selected from the group consisting of alkali metal halide salts, alkaline metal halide salts, alkali nitrates, alkaline metal nitrates, alkali sulphates, alkaline metal sulphates and alkali phosphates, alkaline metal phosphates. In some embodiments, the solute comprises a acid selected from the group consisting of hydrochloric acid, nitric acid, sulfuric acid, phosphoric acid and conjugate ammonium salt acids. In some embodiments, the solute comprises a base selected from the group consisting of alkali metal hydroxides, alkaline metal hydroxides, alkali metal alkoxides, alkaline metal alkoxides, alkali metal carbonates, alkaline metal carbonates, alkali metal carboxylates, alkaline metal carboxylates and amine bases.

In a further aspect, herein provided is a method of selectively recovering a lipophilic target substance, the method comprising: providing a solution comprising the target substance in a lipophilic solvent; combining an adsorbent with the solution, the adsorbent being insoluble in the solution; decreasing a temperature of the solution for associating the target substance with the adsorbent to facilitate binding of the adsorbent with the target substance; and isolating the adsorbent bound with the target substance from the solution; wherein the adsorbent is selected from the group consisting of a cyclic polysaccharide, amylose, maltodextrin, silica gel, polytetrafluoroethylene granules, chloromethylpolystyrene-divinylbenzene resin, polyvinyl butyral resin and acetylated cellulose.

In a further aspect, herein provided is a method of selectively recovering a lipophilic target substance, the method comprising: providing a solution comprising the target substance in a lipophilic solvent; combining an adsorbent with the solution, the adsorbent being insoluble in the solution; evaporating at least a portion of the lipophilic solvent for associating the target substance with the adsorbent to facilitate binding of the adsorbent with the target substance; and isolating the adsorbent bound with the target substance from the solution; wherein the adsorbent is selected from the group consisting of a cyclic polysaccharide, amylose, maltodextrin, silica gel, polytetrafluoroethylene granules, chloromethylpolystyrene-divinylbenzene resin, polyvinyl butyral resin and acetylated cellulose.

In a further aspect, herein provided is a method of selectively recovering a lipophilic target substance, the method comprising: providing a solution comprising the target substance in a lipophilic solvent; providing an adsorption solvent with an adsorbent; combining the solution with the adsorption solvent and with the adsorbent to facilitate binding of the target substance with the adsorbent; and isolating the adsorbent from the solution; wherein the adsorption solvent is less hydrophobic than the lipophilic solvent; the adsorbent is insoluble in the adsorption solvent; the adsorbent is insoluble in the solution; and the adsorbent is selected from the group consisting of a cyclic polysaccharide, amylose, maltodextrin, silica gel, polytetrafluoroethylene granules, chloromethylpolystyrene-divinylbenzene resin, polyvinyl butyral resin and acetylated cellulose.

In some embodiments, providing the solution comprises combining bulk plant material with the lipophilic solvent and separating the bulk plant material from the lipophilic solvent. In some embodiments, combining the bulk plant material with the lipophilic solvent and separating the bulk plant material from the lipophilic solvent comprises: adding the bulk plant material to a distillation apparatus; adding a mixture of the lipophilic solvent and the adsorption solvent to the distillation apparatus; heating the distillation apparatus to evaporate the lipophilic solvent; condensing the lipophilic solvent; and passing the lipophilic solvent through the bulk plant material and into the adsorption solvent. In some embodiments, the bulk plant material comprises material from Cannabis sativa and the target substance comprises a phytocannabinoid. In some embodiments, the lipophilic solvent comprises an organic solvent. In some embodiments, the organic solvent is selected from the group consisting of acetone, acetonitrile, tetrahydrofuran, glycerol, DMSO, dichloromethane and chloroform. In some embodiments, the organic solvent comprises an alcohol. In some embodiments, the alcohol is selected from the group consisting of methanol, ethanol, n-propyl alcohol and isopropyl alcohol. In some embodiments, the organic solvent comprises a hydrocarbon. In some embodiments, the hydrocarbon is selected from the group consisting of n-hexane, butane and propane. In some embodiments, the lipophilic solvent comprises a eutectic solvent. In some embodiments, the eutectic solvent is selected from the group consisting of glucose syrup, and acetic acid mixed with menthol. In some embodiments, the lipophilic solvent comprises an ionic liquid. In some embodiments, the ionic liquid comprises 1-butyl-3-methylimidazolium tetrafluoroborate. In some embodiments, the adsorption solvent comprises water. In some embodiments, the adsorption solvent comprises a chelating agent. In some embodiments, providing the adsorption solvent with the adsorbent comprises combining the adsorption solvent with the adsorbent. In some embodiments, combining the solution with the adsorption solvent and with the adsorbent comprises evaporating at least a portion of the lipophilic solvent prior to combining the adsorption solvent with the solution. In some embodiments, the adsorbent comprises a cyclic polysaccharide and the cyclic polysaccharide comprises cyclodextrin. In some embodiments, the cyclodextrin is selected from the group consisting of α-cyclodextrin, β-cyclodextrin and γ-cyclodextrin. In some embodiments, the adsorbent comprises amylose or maltodextrin. In some embodiments, the adsorbent is cross-linked with a cross-linker selected from the group consisting of hexamethylene diisocyanate, isophorone diisocyanate, 4,4′-methylenebis(phenyl isocyanate) and tolylene-2,4-diisocyanate. In some embodiments, the adsorbent comprises beads that are insoluble in the solution and isolating the adsorbent from the solution comprises filtering the beads out of the solution. In some embodiments, the beads comprise a magnetic substance and isolating the adsorbent from the solution comprises magnetically attracting the magnetic substance. In some embodiments, wherein the adsorbent comprises nanoparticles of a magnetic substance and isolating the adsorbent from the solution comprises magnetically attracting the magnetic substance. In some embodiments, the adsorbent comprises a powder that is insoluble in the solution. In some embodiments, the adsorbent comprises a gel matrix that is insoluble in the solution. In some embodiments, the adsorbent is sequestered within a permeable material. In some embodiments, the permeable material comprises an immersion filter; and isolating the adsorbent from the solution comprises removing the immersion filter from the solution. In some embodiments, the immersion filter carries an identifiable feature for identifying the immersion filter. In some embodiments, the identifiable feature is selected from the group consisting of a radio frequency identification signal, a physical tag, a barcode, colour-coding of the immersion filters, and other visual labelling. In some embodiments, the target substance is ionizable in the solution, and further comprising combining a solute with the solution for competing with the secondary target substance for binding on the secondary adsorbent to dissociate the secondary target substance from the secondary adsorbent into the solution, for recovering the target substance. In some embodiments, the method further comprising increasing a temperature of the adsorbent for dissociating the target substance from the adsorbent and recovering the target substance. In some embodiments, the method further comprising evaporating at least a portion of the adsorption solvent for dissociating the target substance from the adsorbent and recovering the target substance. In some embodiments, the method further comprising combining a dissociation fluid with the adsorbent for dissociating the target substance from the adsorbent and recovering the target substance. In some embodiments, the dissociation fluid comprises a fluid selected from the group consisting of methanol, ethanol, n-propyl alcohol and isopropyl alcohol, other alcohols, acetone, acetonitrile, tetrahydrofuran, glycerol, DMSO, dichloromethane, chloroform, other organic solvents, n-hexane, butane, propane, other hydrocarbons, glucose syrup, acetic acid mixed with menthol, other eutectic solvents, 1-butyl-3-methylimidazolium tetrafluoroborate, other ionic liquids, a heated gas, a pressurized gas, subcritical CO2, other subcritical fluids, supercritical CO2 or other supercritical fluids. In some embodiments, the dissociation fluid has a lower volume than the lipophilic solvent for concentrating the target substance relative to the concentration of the target substance in the solution. In some embodiments, the dissociation fluid is more hydrophobic than the lipophilic solvent. In some embodiments, the method further comprising combining a secondary adsorbent with the solution for binding of the secondary adsorbent with a secondary target substance; combining a secondary adsorption solvent with the solution to facilitate binding of the secondary adsorbent with the secondary target substance; and isolating the secondary adsorbent from the solution; wherein the secondary adsorbent is insoluble in the solution; and the secondary adsorbent carries a structural property or physicochemical property corresponding to a structural property or physiochemical property of the secondary target substance to preferentially adsorb to the secondary target substance over the target substance. In some embodiments, the structural property or physicochemical property is selected from the group consisting of surface chemistry, pore size, cavity dimension, stereoelectronic environment and polarity. In some embodiments, the secondary target substance is a secondary lipophilic target substance, the secondary adsorption solvent is more hydrophilic than the solution and the secondary adsorbent is selected from the group consisting of a cyclic polysaccharide, amylose, maltodextrin, silica gel, polytetrafluoroethylene granules, chloromethylpolystyrene-divinylbenzene resin, polyvinyl butyral resin, acetylated cellulose, branched polysaccharides, linear polysaccharides, oligosaccharides, peptides, proteins, polymerized adducts of amino acids, polyphenolic scaffolds, polymeric isoprenes, fatty acid polyesters, alumina, zeolitic molecular sieves and silicon dioxide. In some embodiments, the secondary adsorbent comprises a cyclic polysaccharide and the cyclic polysaccharide comprises a secondary cyclodextrin. In some embodiments, the secondary cyclodextrin is selected from the group consisting of α-cyclodextrin, β-cyclodextrin and γ-cyclodextrin. In some embodiments, the secondary adsorbent comprises amylose or maltose. In some embodiments, the secondary adsorbent is cross-linked with a cross-linker selected from the group consisting of hexamethylene diisocyanate, isophorone diisocyanate, 4,4′-methylenebis(phenyl isocyanate) and tolylene-2,4-diisocyanate. In some embodiments, the secondary target substance is a hydrophilic target substance, the secondary adsorption solvent is more hydrophobic than the solution and the secondary adsorbent is selected from the group consisting of sand, Amberlite® XAD-4 neutral resin, vermiculite, cellulosic fibres, silicon-coated cellulosic fibres, Fuller's earth, nanoclay hydrophilic bentonite, clay mineral blend, wood pulp, 3 Å molecular sieves, Celite®, Dowex® 1×8 strongly basic resin, silica gel, diatomaceous earth, bleaching earth, activated clays, activated carbons and charcoals, magnesium oxide, alumina, activated alumina, zeolitic molecular sieves, bauxites, silicon dioxide, sodium sulphate, magnesium sulphate, branched and linear polysaccharides, oligosaccharides, peptides and proteins. In some embodiments, the secondary adsorbent comprises beads that are insoluble in the solution and isolating the secondary adsorbent from the solution comprises filtering the beads out of the solution. In some embodiments, the beads comprise a magnetic substance and isolating the secondary adsorbent from the solution comprises magnetically attracting the magnetic substance. In some embodiments, the secondary adsorbent comprises nanoparticles of a magnetic substance and isolating the secondary adsorbent from the solution comprises magnetically attracting the magnetic substance. In some embodiments, the secondary adsorbent comprises a powder that is insoluble in the solution. In some embodiments, the secondary adsorbent comprises a gel matrix that is insoluble in the solution. In some embodiments, the secondary adsorbent is sequestered within a secondary permeable material. In some embodiments, the secondary permeable material comprises a secondary immersion filter; and isolating the secondary adsorbent from the solution comprises removing the secondary immersion filter from the solution. In some embodiments, the secondary immersion filter carries an identifiable feature for identifying the secondary immersion filter. In some embodiments, the identifiable feature is selected from the group consisting of a radio frequency identification signal, a physical tag, a barcode, colour-coding of the immersion filters, and other visual labelling. In some embodiments, combining the adsorbent with the solution takes place sequentially with combining the secondary adsorbent with the solution. In some embodiments, combining the secondary adsorbent with the solution takes place following combining the adsorbent with the solution. In some embodiments, combining the secondary adsorbent with the solution takes place prior to combining the adsorbent with the solution. In some embodiments, combining the adsorbent with the solution takes place simultaneously with combining the secondary adsorbent with the solution. In some embodiments, the secondary target substance is ionizable in the solution, and further comprising combining a solute with the solution for competing with the secondary target substance for binding on the secondary adsorbent to dissociate the secondary target substance from the secondary adsorbent into the solution, for recovering the target substance. In some embodiments, the method further comprising increasing a temperature of the secondary adsorbent for dissociating the secondary target substance from the secondary adsorbent and recovering the target substance. In some embodiments, the method further comprising evaporating at least a portion of the secondary adsorption solvent for dissociating the secondary target substance from the secondary adsorbent and recovering the secondary target substance. In some embodiments, the method further comprising combining a secondary dissociation fluid with the secondary adsorbent for recovering the secondary target substance, wherein the secondary target substance is more soluble in the secondary dissociation fluid than in the solution.

In a further aspect, herein provided is a method of selectively recovering a phytocannabinoid, the method comprising: providing a solution comprising the phytocannabinoid in an organic solvent; providing a hydrophilic solvent with an adsorbent; combining the solution with the hydrophilic solvent and with the adsorbent to facilitate binding of the adsorbent with the phytocannabinoid; isolating the adsorbent from the solution; and wherein the adsorbent is insoluble in the adsorption solvent; the adsorbent is insoluble in the solution; and the adsorbent is selected from the group consisting of a cyclic polysaccharide, amylose, maltodextrin, silica gel, polytetrafluoroethylene granules, chloromethylpolystyrene-divinylbenzene resin, polyvinyl butyral resin and acetylated cellulose.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures, in which reference numerals sharing a common final two digits have equivalent meaning across multiple figures (e.g. the lipophilic solvent vessel 30, 130, 230, 330, 430, etc.).

FIG. 1 is a schematic diagram of the molecular structure of insoluble cyclodextrin polymers;

FIG. 2 is a schematic diagram of a lipophilic compound recovery system;

FIG. 3 is a schematic diagram of the system of FIG. 2 with an insoluble polysaccharide added to the system;

FIG. 4 is a schematic diagram of the system of FIG. 2 with a sample added to the system;

FIG. 5 is a schematic diagram of the system of FIG. 2 with additional lipophilic solvent added to the system;

FIG. 6 is a schematic diagram of the system of FIG. 2 with hydrophilic solvent added to the system;

FIG. 7 is a schematic diagram of the system of FIG. 2 while filtering a binding slurry to recover a lipophilic target compounds;

FIG. 8 is a schematic diagram of the system of FIG. 2 while rinsing a filter with both hydrophobic and hydrophilic solvents;

FIG. 9 is a schematic diagram of the system of FIG. 2 while rinsing the filter with hydrophilic solvent;

FIG. 10 is a schematic diagram of the system of FIG. 2 while flowing lipophilic solvent over the filter to dissolve the lipophilic target compounds;

FIG. 11 is a schematic diagram of the system of FIG. 2 with the contents of a recovery vessel added to the system to repeat the process of FIGS. 5 to 10;

FIG. 12 is a schematic diagram of an insoluble polysaccharide including an insoluble polymer;

FIG. 13 is a schematic diagram of an insoluble polysaccharide including an insoluble polymer;

FIG. 14 is a schematic diagram of an insoluble polysaccharide including a magnetic bead;

FIG. 15 is a schematic diagram of an insoluble polysaccharide including a magnetic bead;

FIG. 16 is a schematic diagram of an insoluble polysaccharide including a magnetic nanoparticle;

FIG. 17 is the molecular structure of an insoluble polysaccharide of including a magnetic nanoparticle;

FIG. 18 is a schematic diagram showing processing of recovered target compounds into downstream products;

FIG. 19 is a schematic diagram of a lipophilic compound recovery system;

FIG. 20 is a schematic diagram of the system of FIG. 19 with an insoluble polysaccharide added to the system;

FIG. 21 is a schematic diagram of the system of FIG. 19 with a sample added to the system;

FIG. 22 is a schematic diagram of the system of FIG. 19 with hydrophilic solvent added to the system;

FIG. 23 is a schematic diagram of the system of FIG. 19 while emptying a binding slurry into a column filter;

FIG. 24 is a schematic diagram of the system of FIG. 19 while washing a loaded column filter with hydrophobic and hydrophilic solvent;

FIG. 25 is a schematic diagram of the system of FIG. 19 while rinsing the loaded column filter with hydrophilic solvent;

FIG. 26 is a schematic diagram of the system of FIG. 19 while rinsing the loaded column filter with hydrophilic and lipophilic solvent;

FIG. 27 is a schematic diagram of the system of FIG. 19 while eluting lipophilic target compounds from the loaded column filter with lipophilic solvent;

FIG. 28 is a schematic diagram of the system of FIG. 19 with the contents of a recovery vessel added to the system to repeat the process of FIGS. 23 to 27;

FIG. 29 is a schematic diagram of a lipophilic compound recovery system;

FIG. 30 is a schematic diagram of the system of FIG. 29 with a polysaccharide added to the system in an immersion filter;

FIG. 31 is a schematic diagram of the system of FIG. 29 with a sample added to the system;

FIG. 32 is a schematic diagram of the system of FIG. 29 with additional lipophilic solvent added to the system;

FIG. 33 is a schematic diagram of the system of FIG. 29 with hydrophilic solvent added to the system;

FIG. 34 is a schematic diagram of the system of FIG. 29 while draining the binding solvent into a storage vessel and moving the immersion filter to a wash tank;

FIG. 35 is a schematic diagram showing storage of the immersion filter of the system of FIG. 29;

FIG. 36 is a schematic diagram of a lipophilic compound recovery system with a plurality of immersion filters;

FIG. 37 is a schematic diagram of a column chromatography system;

FIG. 38 is a schematic diagram of the system of FIG. 37 with a stationary phase added to the system;

FIG. 39 is a schematic diagram of the system of FIG. 37 with a sample added to the system;

FIG. 40 is a schematic diagram of the system of FIG. 37 with hydrophilic solvent added to the system;

FIG. 41 is a schematic diagram of the system of FIG. 37 while eluting the sample with hydrophobic and hydrophilic solvent;

FIG. 42 is a schematic diagram of the system of FIG. 37 while eluting lipophilic target compounds with lipophilic solvent;

FIG. 43 is a schematic diagram of the system of FIG. 37 while eluting lipophilic target compounds;

FIG. 44 is a schematic diagram of the system of FIG. 37 with the contents of a recovery vessel added to the system to repeat the process of FIGS. 41 to 43;

FIG. 45 is a schematic diagram of the molecular structure of a polysaccharide bound to a carbohydrate matrix;

FIG. 46 is a schematic diagram of the molecular structure of a polysaccharide bound to a carbohydrate matrix;

FIG. 47 is a schematic diagram of the molecular structure of a polysaccharide bound to a silica gel;

FIG. 48 is a schematic diagram of the molecular structure of a polysaccharide bound to a silica gel;

FIG. 49 shows mg of CBD captured over time in Example 1;

FIG. 50 shows percent of total CBD captured over time in Example 1;

FIG. 51 shows the percentage of CBD release over time in Example 2;

FIG. 52 shows the percentage of CBD captured over time in Example 3;

FIG. 53 shows the percentage of CBD and CBG captured over time in Example 4;

FIG. 54 shows the percentage of CBD and CBG captured over time when CBD and CBG are in competition in Example 4;

FIG. 55 shows the percentage of vanillin and olivetol captured over time in Example 5;

FIG. 56 shows the percentage capture of vanillin, olivetol, and CBD in Example 5;

FIG. 57 shows percentage capture of CBD in Example 6;

FIG. 58 shows percentage capture of CBD in Example 6;

FIG. 59 shows percentage capture of CBD in Example 6;

FIG. 60 shows percentage capture CBD in Example 6;

FIG. 61 shows the percentage capture of CBD in Example 7;

FIG. 62 shows the percentage capture of CBD in Example 8;

FIG. 63 shows percentage capture over time of CBD in Example 9;

FIG. 64 shows percentage capture of CBD in Example 10;

FIG. 65 shows CBD captured and released in Example 10;

FIG. 66 shows the mg of CBD captured and released in Example 11;

FIG. 67 shows percentage of CBD captured in Example 12;

FIG. 68 shows percentage capture of CBD in Example 13;

FIG. 69 shows mg of CBD captured in Example 13;

FIG. 70 shows percentage CBD captured in Example 14;

FIG. 71 shows mg CBD captured in Example 14;

FIG. 72 shows percentage of CBD captured in Example 15;

FIG. 73 shows mg of CBD captured in Example 15;

FIG. 74 shows percentage of CBD and CBG captured in Example 16;

FIG. 75 shows mg of CBD and CBG captured in Example 16;

FIG. 76 shows mg of CBD captured and released in Example 17;

FIG. 77 shows mg of CBD captured and released in Example 18;

FIG. 78 shows mg of CBD captured, released and released into ethanol versus mg of polymer in Example 19;

FIG. 79 shows mg of CBD captured and released in extract of hops in Example 20;

FIG. 80 shows mg of CBD captured and released in Example 21;

FIG. 81 shows mg of CBD captured and released in Example 22;

FIG. 82 shows mg of CBD captured and released in Example 23;

FIG. 83 shows mg CBD and CBG captured and released in Example 24;

FIG. 84 shows mg CBD captured and released in Example 25;

FIG. 85 shows mg CBD captured and released in Example 26;

FIG. 86 shows mg CBD captured and released in Example 27;

FIG. 87 shows mg CBD released in Example 28;

FIG. 88 shows mg CBD captured and released in Example 29;

FIG. 89 shows mg CBD captured versus eluent in Example 30;

FIG. 90 shows mg CBD captured in Example 31;

FIG. 91 shows mg CBD captured and released in Example 32;

FIG. 92 shows mg CBD captured and released in Example 33;

FIG. 93 shows mg CBD recoverable and recovered in Example 34;

FIG. 94 shows mg CBD captured and released in Example 35;

FIG. 95 shows mg CBD in solution at different time points in Examples 36 and 43;

FIG. 96 shows mg total CBD recovered in Examples 37 and 38;

FIG. 97 shows mg/ml CBD recovered in Example 39;

FIG. 98 shows mg CBD captured and released in Example 40;

FIG. 99 shows mg CBD captured and released in Example 41;

FIG. 100 shows mg CBD captured and released in Example 42;

FIG. 101 shows mg/ml phytocannabinoids captured and released in Example 46;

FIG. 102 shows mg/ml CBD captured and released in Examples 47 and 48;

FIG. 103 shows the chemical structure of xanthumol;

FIG. 104 shows the chemical structure of flavanone;

FIG. 105 shows a time-course HPLC resolution with UV absorption of the reaction mixture before the addition of the cross-linked polymer in Example 50;

FIG. 106 shows a time-course HPLC resolution with UV absorption of the reaction mixture after filtration and flushing of the cross-linked polymer Example 50.

FIG. 107 shows mg/ml CBD captured and released in Example 51;

FIG. 108 is a schematic diagram of a system with hydrophilic solvent and the adsorbent added to the system;

FIG. 109 is a schematic diagram of the system of FIG. 108 with a sample and hydrophilic solvent added to the system;

FIG. 110 is a schematic diagram of the system of FIG. 108 while filtering a binding slurry to recover a lipophilic target compounds;

FIG. 111 is a schematic diagram of the system of FIG. 108 while rinsing a filter with both hydrophobic and hydrophilic solvents;

FIG. 112 is a schematic diagram of the system of FIG. 108 while rinsing the filter with hydrophilic solvent;

FIG. 113 is a schematic diagram of the system of FIG. 108 while flowing lipophilic solvent over the filter to dissolve the lipophilic target compounds; and

FIG. 114 is a schematic diagram of the system of FIG. 108 with the contents of a recovery vessel added to the system to repeat the process of FIGS. 108 to 113.

### DETAILED DESCRIPTION

Generally, the present disclosure provides a method for selective recovery of lipophilic compounds through application of insoluble adsorbents. The method includes capturing and releasing lipophilic target substances. The lipophilic target substances may include natural product classes from plant matter, including polyphenolics, terpenoids and phytocannabinoids. The method may include multiple insoluble adsorbents for other lipophilic compounds, or insoluble adsorbents for hydrophilic compounds. Physical separation of insoluble adsorbents, such as through immersion filters, may facilitate recovery of the compounds. Adsorption and dissociation drivers may include addition or evaporation of lipophilic or hydrophilic solvents, addition of solutes, changes in temperature or other suitable drivers.

In view of the previously described work and related shortcomings, there is motivation to provide an improved approach to capturing plant metabolites. The method and system provided herein applies and expands upon the potential for capturing lipophilic target compounds by adsorption with insoluble adsorbents through molecular interactions between guest lipophilic small molecules and the adsorbent host macromolecular scaffold. Cyclodextrins contained within polymeric matrices have been used for phenol decontamination from water but have not been utilized in the extraction of plant metabolites from plant matter. The method described herein applies molecular recognition and non-covalent bonding interactions within guest-host inclusion complexes of adsorbents and lipophilic compounds.

The method and system provided herein may involve multiple adsorbents, which may be physically separated adsorbents that bind to specific components within a plant extract, and providing the flexibility and control of removal, addition, or substitution of individual adsorbents. The use of a plurality of adsorbents housed in individual immersion filters, each with an affinity for different target compounds, may provide a means to simultaneously bind, separate, and thus purify compound mixtures.

Porous substances with structural or surface characteristics may be tailored to afford selective or specific affinity toward the physicochemical, structural, or molecular weight features of certain compounds, or classes of compounds, within a cannabis extract. High binding specificity may be demonstrated for a range of phytocannabinoids beyond those described herein. For example, adsorbents with a high affinity for specific phytocannabinoids or families of phytocannabinoids, may be applied to polymers with distinct physicochemical, structural or molecular weight properties that suited to that of certain compounds, or classes of compounds, within a cannabis extract. Principles of combinatorial chemistry may be adapted to create a gradient of adsorbents for solid-phase gradients, or dissociation conditions for liquid-phase gradients, or of other variables for gradient capture. For example, an array of wells or multiple immersion filters may be contacted with a solution comprising a target substance. The wells in the array of wells or the multiple immersion filters may each contain or house the same of different adsorbent.

Filtration of insoluble polymeric material such as cellulose and related biopolymers may be applied to a plant extract, leaving the target molecule in an organic solvent. The adsorbent may be added to the solvent to facilitate capture of a target molecule. Slow addition of hydrophilic solvents, such as water, gradually decreases the solubility of the lipophilic target compounds and provides a driving force for entry into the adsorbent cavity due to low solubility in the resultant mixture of organic solvent and hydrophilic solvent.

The choice of initial organic solvent may be tailored to accommodate the solubility of a given natural product class to be extracted as well as the insolubilities of various unwanted plant materials such that unwanted material remains within the plant biomass and is separated during the filtration process. The choice of initial solvent may be governed not only by the ability to exclude plant metabolites from biomass but also due to the physicochemical properties of a resulting solvent-water mixture that are crucial for highly specific encapsulation into the polymer cavities.

Deep eutectic solvents or ionic liquids may be used to solubilize lipophilic target compounds from plant biomass and biopolymers. Upon addition of water, intermolecular forces governing the properties of these unique solvents may be heavily disrupted, and their ability to solubilize target molecules lowered, driving lipophilic target compounds into the cyclic polysaccharide cavity or otherwise binding with the adsorbent. A solvent-water mixture may include an aqueous solution with an agent that destroys the chemical structure of unwanted plant metabolites or constituents such as chlorophyll. For instance, aqueous-soluble chelating agents can bind to the magnesium atom of chlorophyll architecture thereby essentially denaturing the chlorophyll, transforming the molecular structure from one that may bind to a particular adsorbent host into one that does not compete for encapsulation with the target molecule of interest.

Dissociation fluids may include any solvent capable of disrupting the intermolecular forces responsible for tight guest-host binding of the target compound within the adsorbent and of solubilizing the plant metabolite of interest upon release. Dissociation fluids for recovering the lipophilic target substances may include volatile non-toxic solvents such as ethanol that can be easily removed, non-volatile solvents such as dimethyl sulfoxide (“DMSO”) that can be used directly as vehicles for delivery of plant metabolites into cell line assays, supercritical fluids such as carbon dioxide, or the application of heat with concomitant trapping of vaporized plant metabolites, such that solvent-free isolates can be attained following return to atmospheric pressure and removal of the gaseous medium.

Solvents used during extraction or release protocols may be recycled by means of closed-loop systems that restrict solvent evaporation and permit re-entry and re-use of solvents for subsequent extraction procedures, thereby reducing waste and cost.

Following an extraction-release protocol, device cleaning protocols may be used to remove unremoved plant metabolites from tanks, columns or other capturing devices and apparatus. This procedure permits re-use of the capturing device and of the adsorbent during multiple extraction cycles.

Adsorbents for Hydrophobic and Hydrophilic Target Compounds

Adsorbents may display selectivity for classes of compounds, or specificity for individual compounds, according to, for example, surface chemistry, pore size, cavity dimensions, stereoelectronic environment, or complimentary polarity of the adsorbent material matrix, and may be selected based on these features, or other structural and physicochemical properties, with or without further surface or chemical modification, to bind a target of interest or range of targets.

Some examples of adsorbents that may be applied to adsorption of lipophilic compounds include cyclic polysaccharides, which may include cyclodextrins, which may include α-cyclodextrin, β-cyclodextrin or γ-cyclodextrin. Additional examples of adsorbents that may be applied to adsorption of lipophilic compounds include silica gel, cyclodextrin-IPI, cyclodextrin-MPI, PTFE Granules, oligosaccharides, non-cyclic polysaccharides, amylose-HDI, Merrifield PVB/DVB resin, cyclodextrin-TDI, maltodextrin-HDI, cyclodextrin-HDI, and cyclodextrin-HDI with brine. Additional examples of adsorbents that may be applied to adsorption of lipophilic compounds include biopolymers, with or without synthetic modification, such as branched and linear polysaccharides, oligosaccharides, acetylated cellulose, peptides, proteins, polymerized adducts of amino acids (e.g. melanin, etc.), polyphenolic scaffolds (e.g. lignin, suberin, etc.), polymeric isoprenes (e.g. rubber, etc.), and fatty acid polyesters (cutin, cutan, etc.), including after grafting, cross-linking, blending or coating to impart selected solubility properties. Additional examples of adsorbents that may be applied to adsorption of lipophilic compounds include alumina, zeolitic molecular sieves and silicon dioxide.

Some examples of adsorbents that may be applied to adsorption of hydrophilic compounds include sand, Amberlite® XAD-4 neutral resin, vermiculite, cellulosic fibres, silicon-coated cellulosic fibres, Fuller's earth, nanoclay hydrophilic bentonite, clay mineral blend, wood pulp, 3 Å molecular sieves, Celite®, and Dowex® 1×8 strongly basic resin. Some examples of porous adsorbents that may be applied to adsorption of hydrophilic compounds include silica gel, diatomaceous earth, bleaching earth, activated clays, activated carbons and charcoals, magnesium oxide, alumina, activated alumina, zeolitic molecular sieves, bauxites and silicon dioxide. Some examples of non-porous adsorbents that may be applied to adsorption of hydrophilic compounds include sodium sulphate and magnesium sulphate. Some examples of adsorbents that may be applied to adsorption of hydrophilic compounds include branched and linear polysaccharides, oligosaccharides, peptides and proteins.

Biopolymers may be applied to use as insoluble adsorbents in their native form if insoluble or rendered insoluble by, for example, grafting, cross-linking, blending, or coating, including their use within composites of synthetic origin, such as polysaccharide and oligosaccharide structures or lignin architectures, biocompatible excipient-type materials, or proteins.

Conventional applications of non-polymeric cyclodextrins within the pharmaceutical industry have centered on their ability to form inclusion complexes with lipophilic drugs. Cyclodextrin-based inclusion complexes facilitate preparing aqueous soluble powdered forms of otherwise highly insoluble drug molecules. These formulations may enhance shelf life or prolongs their stability in vivo during drug administration. Cyclodextrin-inclusion complexes are applied as vehicles for drug delivery whereby powdered material can be pressed into tablets to provide reliably dosed drugs that are not soluble in aqueous solutions or that may otherwise only be available in liquid form. Challenges associated with administration of liquid drugs include dosing difficulties, shelf life irregularities, and limitations associated with administration method.

Cyclodextrins are a family of cyclic oligosaccharides comprised of repeating glucose subunits joined by α-1,4-glucosidic bonds. Cyclic oligosaccharides may include different repeating subunits or alternative linking bonds. For instance, cyclic oligosaccharides comprised of the same monosaccharide, alternating different monosaccharides, or completely distinct monosaccharides contained within a cyclic architecture that is either comprised of glycosidic bonds or formed by means of alternative cyclisation modes known in synthetic organic chemistry. The parent α-1,4-glucose-based cyclodextrins may be formed using six, seven, or eight repeating sugars subunits and are described as α-, β-, and γ-cyclodextrins, respectively.

The macromolecular scaffold cyclodextrins and other cyclic oligosaccharides may be represented as a cone shaped architecture whereby the 6-position hydroxyl groups of glucose subunits are directed toward the narrow region of the cone and the 2-, and 3-hydroxyl groups are positioned near the broader opening. The inner rim of the cyclic oligosaccharide is notably more lipophilic than the outer rim. The inner and outer rims of the cone or torus shaped host are more hydrophilic in behaviour. Structural analysis of complexes between adsorbents and lipophilic target substances may be applied to rationalize why lipophilic molecules prefer to occupy the inner portion of these host scaffolds with the hydroxyl groups contributing to hydrogen-bonding networks with water-dominant solvent molecules to confer aqueous solubility of the guest-host inclusion complex. Physicochemical characterization of inclusion complexes in solid state using X-ray data, or in solution using nuclear magnetic resonance (“NMR”) or other spectral analyses, demonstrates that guest molecules may offer hydrogen-bonding, dipole-dipole, and Van der Waals interactions with the host polysaccharide, thereby driving complex formation and energetically supporting continued complexation when present in aqueous media.

Polysaccharide mixtures have been used to improve the water solubility of phytocannabinoids and whole plant extracts of cannabis. Applications of cyclic polysaccharides, such as cyclodextrins, within the context of phytocannabinoid chemistry, have also focused on the solubilisation of such compounds. Varied solubilisation efficacies have been noted for certain phytocannabinoid-cyclodextrin partners, demonstrating some structure-dependent cyclodextrin-phytocannabinoid interactions. In the case of a THC-β-cyclodextrin adduct, the non-covalent interactions responsible for efficient encapsulation into the cyclodextrin core have been partially studied by means of NMR analysis, demonstrating that modifications to said non-covalent interactions may ameliorate this molecular recognition and that tuning of the cyclodextrin architecture may provide selectivity for phytocannabinoids, flavanones, other classes of polyphenolics, or various other metabolites of interest.

Monosaccharides and polysaccharides other than cyclodextrin have been shown to influence of the aqueous solubility of phytocannabinoids and whole plant extracts, demonstrating unique molecular interactions between oligosaccharides and phytocannabinoids responsible for the solubilising behaviour.

Cyclodextrin-based polymers have been used for the capture and removal of phenolic compounds, with some selectivity for certain phenol derivatives, from aqueous media, lipophilic media, and from plant material.

Cyclodextrins have been widely employed as vehicles for administration of small molecules in food additives or in the pharmaceutical industry. However, cyclodextrins have had limited application as capture agents for plant metabolites, including in chromatography for bulk selective recovery of lipophilic target compounds. Silica-bound cyclodextrins have found application in analytical technologies on monolayers. In the method provided herein, cyclodextrins are used for selective recovery of phytocannabinoids and other compounds from broadly inclusive plant extracts. Polymer-bound cyclodextrins, related cyclic polysaccharides or other adsorbents facilitate selective recovery and physical separation of specific plant metabolites from a large mixture of plant constituents, such as would be found in an initial crude plant extract following common extraction methods. In a chromatography column for example, polymeric material facilitates fast flow rates and extraction from wet plant matter.

Cyclodextrin-containing polymers have not been previously employed in chromatographic applications for recovery and purification of the lipophilic compounds, for instance the use of such polymeric material to pack chromatography columns. Previous applications in chromatography have been limited to HPLC or other analytic techniques. In such applications, mesoporous cyclodextrin-containing material has been created by covalently appending monomeric cyclodextrins to silica gel.

The inside diameter (“ID”) of cyclic polysaccharides or other adsorbents applied in the method provided herein define the upper size limit of target molecules that can be encapsulated, hence physically separated and isolated. α-cyclodextrin (ID=0.45 nm), β-cyclodextrin (ID=0.60 nm), and γ-cyclodextrin (ID=0.75 nm) each present distinct size restrictions to target molecules, such as specific plant metabolites within a whole plant extract. Target molecules can be divided by their ability to enter and remain within the cavity of the host polysaccharide according to molecular size.

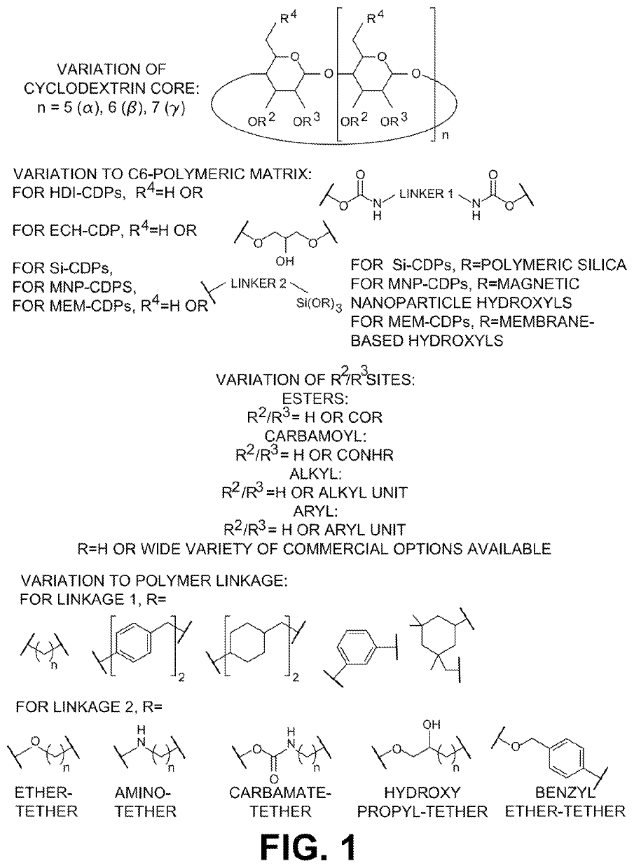

The adsorbent may be applied as insoluble polymeric material, such as where the cyclic polysaccharide or other sugar is reacted with a cross-linking agent. The cross-linking agent may include diisocyanates depicted in FIG. 1, such as hexamethylene diisocyanate (“HDI”). Such polymers can be added to crude extracts of plant material derived from conventional organic solvents, water, deep eutectic solvents, ionic liquids, or a mixture thereof, following filtration of plant debris. Attenuation of the hydrophobicity of the solvent mixture promotes selective retention of lipophilic target compounds in the adsorbent, for example by the slow addition of water to an ethanol plant extract, and filtration of the insoluble polysaccharide permits physical exclusion of target metabolites from the solvent. Suspension of metabolite-bound polymers in more user-friendly solvent mixtures, such as the use of a supercritical carbon dioxide system, or the application of heat, promotes selective release of captured lipophilic target substances from the adsorbent and into a hydrophobic recovery solvent.

The adsorbents may be appended to magnetic nanoparticles, insoluble magnetic beads or powders that can be added to crude extracts of plant material derived from conventional organic solvents, water, deep eutectic solvents, ionic liquids, or a mixture thereof, following filtration of plant debris. The magnetic nanoparticle or magnetic bead can be attached to the adsorbent using a variety of synthetic methods or alternative approaches applied in chemical elaboration of nanoparticles or functional magnetic material preparation. The adsorbent may be separated from the solvent mixture by magnetic separation. Suspension of metabolite-bound magnetic nanoparticles in a more user-friendly solvent mixture, or the application of heat, promotes release of captured metabolites in highly purified or enriched form.

The adsorbent may be bound to a chromatography medium or coated onto a surface such as silica gel. The silica-bound adsorbent may be prepared using synthetic methods or using alternative methods applied in preparation of silica-bound organic substrates. The chromatography medium may be used in substitution of conventional silica gel for the purpose of chromatographic separation of target molecules, such as hydrophobic plant metabolites, from each other, from unwanted plant material, or from the solvent mixture itself.

The adsorbent may be embedded in a chromatography medium such as a chromatography column for use with high-pressure liquid chromatography (“HPLC”), supercritical fluid chromatography, or related techniques. The adsorbent may be used in an analogous manner to conventional chromatography columns by adding a solvent including the target molecule and other compounds to the column and eluting with a gradient, or step-gradient, elution of varying polarity to remove unwanted compounds, such as plant metabolites that adhere less strongly to the adsorbent and retaining the target molecules that bind most strongly to the adsorbent. Elution using a solvent that disrupts this balance of polymer-bound vs solution-phase occupancy permits selective elution and capture of target molecules.

Use of Multiple Adsorbents

Some adsorbents may be applied for adsorbing phytocannabinoids, terpenophenolics or other lipophilic target compounds, and other adsorbents may be applied for adsorbing contaminants or impurities. adsorbents with a high affinity for hydrophobic, phenolic, non-polar, phytocannabinoids or other terpenophenolic compounds may be used for isolating particular lipophilic target compounds. Hydrophilic, metal-coordinating, polar, and pigment-binding adsorbents may be used for decontamination and impurity removal purposes, or other targeting of hydrophilic compounds.

Principles of combinatorial chemistry may be adapted to create a gradient for gradient capture. For example, an array of wells or multiple immersion filters with different adsorbents may be contacted with a solution comprising a target substance. The wells in the array of wells or the multiple immersion filters may each contain or house the same of different adsorbent.

Immersion Filters

An immersion filter containing an adsorbent may be added to, or removed from, the modified extractant solution once adsorption has occurred to an adsorbent in contact with the solution phase. Additional adsorbents may be structurally different from those previously exposed to the solution or may be of the same kind previously used comprising either reused or unused material. Multiple immersion filters may be applied to provide multiple different adsorbents for different hydrophilic and hydrophobic target compounds. One or more of the immersion filters may be easily moved from a solution, temperature, pressure or other set of drivers that facilitates adsorption to a solution, temperature, pressure or other set of drivers facilitates desorption.

The method and system may be applied to adsorb a first target compound with a first adsorbent and a second target compound with a second adsorbent. For example, a first adsorbent may be a polysaccharide derivative, for example a polyurethane derived from beta-cyclodextrin, and may be bound to or enclosed within a first immersion filter. The first immersion filter may be exposed to a solution including the first target compound, the second target compound and other target compounds. After binding of the first target compound with the first adsorbent, the first immersion filter may be withdrawn from the solution. Subsequently, a second immersion filter including a second adsorbent bound to or enclosed within the second immersion filter may be applied to the solution. The second immersion filter may also include the first adsorbent to adsorb and recover additional amounts of the first target compound. The second immersion filter may alternatively or additionally include a second adsorbent that is directed to recovering a second target molecule. The second adsorbent may be a distinct polysaccharide derivative or may be an adsorbent other than a polysaccharide. Additional immersion filters may also be used beyond the first and second immersion filters. The approach of using multiple immersion filters may also be applied to a system with multiple adsorbents added to the same adsorption tank, or by passing the solution through multiple chromatography columns with immobilized adsorbents.

Adsorption and Dissociation Drivers

Solvent mixtures may be used to attenuate the solubility and relative affinity for binding to the adsorbent of lipophilic target substances from a heterogeneous mixture, such as a particular hydrophobic plant metabolite from a whole plant extract. Some solvents will assist in the binding and selective isolation of particular plant metabolites from the plant biomass while leaving unwanted material in solution or degrading unwanted molecules to prevent binding to the adsorbent that has been deployed for the isolation of the lipophilic target substances. Examples of such degradation include addition of ethylenediaminetetraacetic acid (“EDTA”), ethylene glycol-bis(2-aminoethylether)-tetraacetic acid (“EGTA”) or other chelating agents to the solution to bind Mg²⁺ coordinated to chlorophyll, or other metal ions coordinated to other molecules, degrading the chemical structure of such molecules and limiting binding of such molecules to the adsorbent. Solvent mixtures may be applied whereby an organic solvent, deep eutectic solvent or ionic liquid is applied to dissolve the lipophilic target compound. Lowering the hydrophobicity of the solvent, such as by adding water, adding a hydrophilic solvent, or adding a salt, favours binding of the target molecule to the adsorbent rather than staying in solution, facilitating selective retention of the target compounds.

Adsorbents may be used in conjunction with subcritical or supercritical fluids, including heated liquids, to either capture target compounds from solution under such conditions or used to elute target compounds substances from adsorbent-target complexes such that conventional solvents, and residues thereof, may be avoided in purification.

Adsorbents may be used in the workflow of near-critical fluids to adsorb, separate, or purify target compounds from a plant extract. Such workflows may comprise a stream of fluid passed through plant material and passed through impurity-removing adsorbents (such as activated charcoal), but not through adsorbent used to capture and retrieve compounds of interest. Likewise, elution of compounds of value that may be adsorbed onto or absorbed into to a porous matrix using near-critical fluids as solvent. As such, target substances may be physically bound or removed from a subcritical or supercritical fluid to facilitate recovery at a later stage.

Target compounds within a cannabis extract composition may be induced to bind a porous adsorbent by modification to solution phase conditions that approach the saturation point of said compounds, without precipitation into the vessel or solution.

The addition of a large excess of an insolubilizer, in which a target compound is poorly insoluble, to a solution of the target in the presence of the porous adsorbent may lead to rapid precipitation of targets from solution as an amorphous, solid or oily composition that may be unstable or impractical to recover if added to the solution alone. In the presence of a suitable adsorbent, favorable interactions between target compounds and the adsorbent surface structure, including cavities, pores, or internal substructures, may provide the energetic driving force that prohibits direct expulsion from solution and favors target compound residence within the adsorbent matrix.

Adsorbent replacements may be performed, for example, under particular liquid phase conditions in which one targets binds to a given adsorbent, but a different target does not. Modification of solution phase conditions by addition or evaporation of lipophilic or hydrophilic solvents, addition of solutes or changes in temperature may provide an environment suitable to promote binding of the previously unbound target to the same adsorbent. In this manner, an adsorbent with potential affinity for multiple targets may be used to selective bind individual components by exploiting liquid phase gradients using appropriate intermittent adsorbent washings or replacements with unused adsorbent.

Adsorbent substitutions may be carried out such that unwanted substances adsorbed earlier in the course of a liquid phase gradient may be discarded by removal of an adsorbent. Substitution with of adsorbents may be advantageous due to the removal of potentially competing, and thus contaminating, components capable of binding new adsorbents under previous conditions. In this format, adsorbents used and removed prior to addition to target-binding adsorbents may be considered sacrificial adsorbents.

Adsorbent materials may include reactive functionalities that chemically modify the target compounds or chemically modify the liquid phase, such as through protonation. Chemical modification of solution phase conditions or the structure of targets may alter binding affinities, or relative binding affinities, of specific target compounds, thereby promoting adsorption or desorption. For instance, basic ion exchange resins may be used to lower the pH of the liquid phase and deprotonate acidic targets. The resulting anionic target may then be recovered as the adsorbent-target salt that is removed from the mixture and eluted under desalting conditions.

Upon addition of the extractant solution to adsorbent conditions may be modified to induce adsorption of target compounds. This may allow extractions to be performed at elevated temperatures, for example, as part of modified Soxhlet extractions or with other distillation apparatus. By inclusion of one or more adsorbents inside the still pot, each contained within separate immersion filters with affinities for certain lipophilic target compounds, hydrophilic impurities or other compounds, a Soxhlet process or other distillation process may be applied.

Allowing the mixture to cool gradually under pressure may provide an appropriate stimulus for adsorption of lipophilic compounds, in view of known heat effects on hydrophobic interactions.

As cooling occurs, solubility of lipophilic compounds may decrease and reach saturation leading to precipitation or ‘oiling out’, usually requiring organic-aqueous solvent extractions to separate. Immersion filters or other housing for the adsorbents may instead be used to bind substances that resume insolubility at ambient temperature, allowing compartmentalization of immersion filters according to target affinity, and elution from adsorbents using pressurized fluids, such as liquid carbon dioxide, to ultimately liberate separated compounds without any exposure to solvent residues.

Deep eutectic solvents or ionic liquids may be used to solubilize lipophilic target compounds from plant biomass and biopolymers. Upon addition of water, intermolecular forces governing the properties of these unique solvents may be heavily disrupted, and their ability to solubilize target molecules lowered, driving lipophilic target compounds into the cyclic polysaccharide cavity or otherwise bound to the adsorbent. A solvent-water mixture may include an aqueous solution with an agent that destroys the chemical structure of unwanted plant metabolites or constituents such as chlorophyll. For instance, aqueous-soluble chelating agents can bind to the magnesium atom of chlorophyll architecture thereby essentially denaturing the chlorophyll, transforming the molecular structure from one that may bind to a particular adsorbent host into one that does not compete for encapsulation with the target molecule of interest.

Solution phase modifications may be used to attenuate target solubilities and adsorbent affinities toward individual compounds. Such modifications may include temperature variations, solvent evaporation, and addition of solvents or solvent mixtures. Embodiments can also include addition of solid substances, in solution or non-solvated, such as ionic salts, chelating agents, soluble polymeric, oligomeric, or monomeric substances, or mixtures thereof, for inhibiting or inducing precipitation of individual compounds. For example, addition of aqueous sodium chloride to an ethanol extract may enhance polarity differentials to instigate saturation of lipophilic compounds, or the addition of aqueous lactose solutions may partially solubilize some lipophilic compounds over others.

Solvent removal or solvent switches may be performed wherever necessary during the separation process. For instance, a broad-spectrum binding adsorbent may be added in excess quantity to an ethanol extract, preferably using a immersion filter but potentially without containment, and may be followed by an excess quantity of an insolubilizer such as brine. Upon complete adsorption, the adsorbent may be separated from the remaining liquid, rinsed and dried of any remaining fluids or solvents. The bound mixture of chemical substances may then be desorbed from the insoluble adsorbent by submersion in or rinsing with a preferred solvent. Separation of the immersion filter and retrieval of the adsorbent yields the mixture of extracted compounds as a solution in a different solvent from which they were originally extracted, without necessitating evaporation equipment.

Target compounds adsorbed onto the adsorbents may be retrieved by application of a desorption driving force. Driving forces that promote dissociation of bound target compounds from the adsorbent may include application of heat, application of vacuum, exposure to a lipophilic solvent or mixture of lipophilic solvents (e.g. ethanol, hydrocarbons, halogenated hydrocarbons, polar aprotic solvents, glycerin, propylene glycol, medium chain triglycerides, coconut oil, olive oil, general nut oils, general seed oils, hemp oil, liquid CO₂, other pressurized gases).

Liquid dissociation fluids may include any solvent suitable for disrupting intermolecular forces responsible for tight guest-host binding of the target compound to the adsorbent and of solubilizing the target compound upon release. Dissociation fluids for recovering the lipophilic target substances may include volatile non-toxic solvents such as ethanol that can be easily removed, non-volatile solvents such as DMSO that can be used directly as vehicles for delivery of plant metabolites into cell line assays, supercritical fluids such as carbon dioxide, or the application of heat with concomitant trapping of vaporized plant metabolites, such that solvent-free isolates can be attained following return to atmospheric pressure and removal of the gaseous medium.

Submersion of the adsorbent in a volume of ethanol may dissociate the lipophilic target substances from the adsorbent.

Lipophilic target substances may be desorbed from the adsorbent by heated gases, subcritical fluids or supercritical fluids. Liquid carbon dioxide may be passed through the adsorbent bound to the lipophilic target substances and diverting the pressurized liquid solution to a different vessel, thus desorbing the lipophilic target substances from the adsorbent. Exposure of lipophilic target substances bound to the adsorbent by a gas stream, such as hydrocarbons, carbon dioxide or other suitable gases may provide lipophilic target substances collected in neat form without residual solvent.

Solid phase gradients, as distinct separation mechanisms or used in conjunction with liquid phase gradients, may be achieved by repeated substitutions of housed adsorbents with properties varying along a continuum, such as pore size or hydrophobicity.

Fractional adsorption may be performed by sequentially removing adsorbents and replacing with structurally similar adsorbents that display, preferably incremental, changes in a particular physicochemical characteristic thus establishing a solid phase gradient. For instance, after a portion of the solution phase mixture has adsorbed onto the adsorbent, the immersion filter can be removed from the mixture and replaced with a new mesh filter containing an adsorbent with a tailored adjustment in hydrophobic property or pore size.

By employing sequential relays of fractional adsorption, the mixture may be separated entirely by modifying the solid phase alone or in conjunction with liquid phase gradients enacted simultaneously or as discrete steps. For instance, fractional adsorption may be performed, and products desorbed from the insoluble adsorbent into ten separate vessels according to the pore size of material used, hence corresponding to target molecular weight. Next, using a single adsorbent for all ten fractionated mixtures, a liquid phase gradient may be established as previously outlined with intermittent adsorbent washing at set intervals. This embodiment may provide two-dimensional mixture deconvolution by, for example, fractionation first according to molecular weight and subsequent separation according to polarity.

Fractional elution of substances bound to an adsorbent may be realized by tailoring desorption conditions to effect partial removal of bound substances. For instance, following binding of solution phase components, withdrawal of the immersion filter from the liquid may be followed by submersion in a separate vessel containing a solvent mixture that preferentially desorbs one substance, or group of substances, over other substances that remain adsorbed. After a set time, an immersion filter or other easily retrieved insoluble adsorbent may be removed from this solution now containing substances partially desorbed from the adsorbent and submerged in another solvent that promotes desorption of some or all remaining materials.

Gradient capture may allow for a more precise level of separation of a target substance from solution, for example, if two compounds share a similar chemical or physical property and both are in solution, an adsorbent gradient may be created with respect to the same physical or chemical property to improve capture yield.

Solvents Including Lipophilic Target Compounds

An extractant solution may include a liquid solvent or mixture of solvents containing a lipophilic target compound is provided (e.g. a phytocannabinoid, terpenoid, etc.).

In preparation for extraction plant material may be freshly harvested, dried, frozen or decarboxylated. Pre-extraction treatments may be performed to modify plant metabolites or to expedite release from botanical sources, for instance, by chemical or enzymatic processing. Where deemed necessary, ultrasonic, thermal, microwave, or mechanical agitation may be applied to improve physical extrusion of target compounds by physically breaching micellular architectures or through weakening metabolite-protective botanical cells and tissues.

A starting extractant solutions may contain substances obtained directly following an extraction method or following partial separation of one of more substances. Starting solution conditions may begin with a homogeneous extractant solution that but may be filtered to remove unwanted settled material and may require addition of appropriate solvent to homogenize the mixture completely. If extractants are obtained from biomass in concentrated form, solvent may be used to create a solution containing the extractant. An inverse starting protocol may be established by adding extractant solution directly to at least one adsorbent.

Lipophilic adsorbents may be contacted with an extractant solution comprising heated or subcritical water containing a mixture of hydrophilic and lipophilic compounds. Hot aqueous extractant may be contacted with one or more appropriate adsorbents prior to pressure discharge.

Spent biomass may be physically separated from the extractant solution, for example, by filtration or centrifugation, or may remain during subsequent steps if accommodated within a liquid-permeable immersion filter.

The target compounds may be present as a mixture of chemical substances also comprising contaminating substances (e.g. heavy metal ions, pesticides, etc.). The target compounds may be present as a mixture of chemical substances such as phytochemicals (e.g. phytocannabinoids, terpenoid, flavonoids, prenylated phenols, chlorophylls, waxes, lipids, macromolecules, etc.). Liquid phase homogeneity and optimal viscosity may be ensured by the addition of one or more solvents in addition to those employed to isolate compounds from the biomass.

Ethanol may used as a solvent in the extractant solution and spent biomass may removed by filtration to yield an ethanolic extract containing target bioactive substances, unwanted bioactive substances, impurities, and contaminants. Solvents other than ethanol that are appropriate for plant extractions may also be used. The solvent may be selected based on different dielectrics and other properties. The solvent may be in gaseous or solid state under ambient conditions but be driving into the liquid state by application of temperature or heat during or after extraction from the plant biomass. The solvent may also comprise a fluid in the supercritical state.

An extractant solution may be prepared from an extract obtained using subcritical or supercritical carbon dioxide, subcritical water, volatile hydrocarbons, alcohols other than ethanol, conventional organic solvents, ionic liquids, deep eutectic solvents and mixtures thereof. The extract liquid may be comprised of a pure solvent or may also be composed of substances dissolved within the solvent that may modify extraction efficiency or attenuate solvent utility such as ionic salts, chelating agents, pH buffers, proteins, or sugars. Substances known to modify the surface tension of liquids in contact with substances of the same state or different phase, may be contained within the extractant liquid, such as surfactants, detergents, wetting agents, emulsifiers, foaming agents, and dispersants.

The choice of initial solvent may be tailored to accommodate the solubility of a given natural product class to be extracted as well as the insolubilities of various unwanted plant materials such that unwanted material remains within the plant biomass and is separated during the filtration process. The choice of initial solvent may be governed not only by the ability to exclude plant metabolites from biomass but also due to the physicochemical properties of a resulting solvent-water mixture that are crucial for highly specific encapsulation into the polymer cavities.

Solvents used during extraction or release protocols may be recycled by means of closed-loop systems that restrict solvent evaporation and permit re-entry and re-use of solvents for subsequent extraction procedures, thereby reducing waste and cost.

Following an extraction-release protocol, device cleaning protocols may be used to remove unremoved plant metabolites from tanks, columns or other capturing devices and apparatus. This procedure permits re-use of the capturing device and of the adsorbent during multiple extraction cycles.

Lipophilic Compound Recovery System

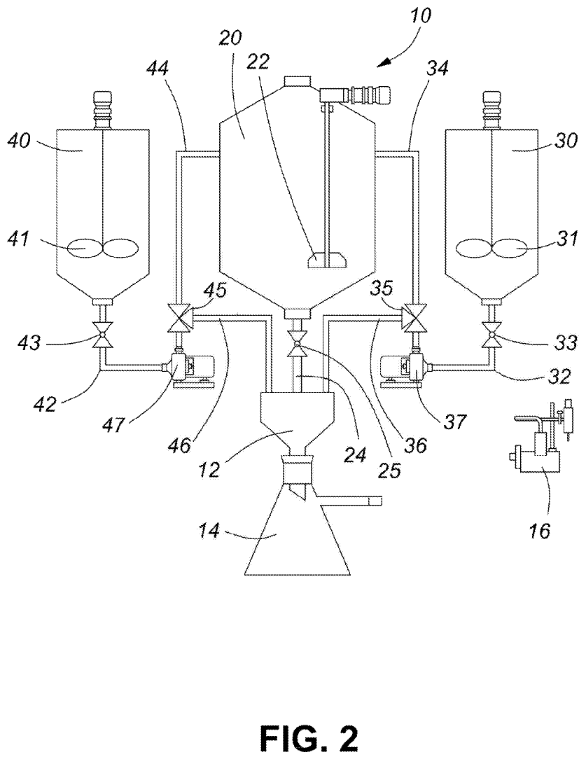

FIG. 2 shows a lipophilic compound recovery system 10. The system 10 includes a slurry vessel 20. A filter 12 is in fluid communication with the slurry vessel for receiving fluid from the slurry vessel 20 and filtering material out of the fluid. The filter 12 is shown as a filter funnel but any suitable filter may be applied (e.g. a sintered glass filter, polytetrafluoroethelyne membrane filter, etc.) A recovery vessel 14 is in fluid communication with the filter 12 for receiving filtrate that passes through the filter 12. The recovery vessel 14 is shown as a Büchner funnel, but any suitable recovery vessel 14 may be applied (e.g. a flask, Erlenmeyer, round-bottom flask, beaker, test tube, etc.). A processing system 16 may be in fluid communication with the recovery vessel 14 for processing target molecules captured using the filter 12. The slurry vessel 20 is in fluid communication with a lipophilic solvent vessel 30 for receiving lipophilic solvent from the lipophilic solvent vessel 30. The slurry vessel 20 is in fluid communication with a hydrophilic solvent vessel 40 for receiving hydrophilic solvent from the hydrophilic solvent vessel 40.

Each of the slurry vessel 20, the lipophilic solvent vessel 30 and the hydrophilic solvent vessel 40 may be any suitable fluid vessel appropriate for the size, scale and application of the system 10 (e.g. a tank, pressure-rated tank, etc.).

The lipophilic solvent may be any suitable lipophilic solvent in which a target substance is soluble, in which an insoluble polysaccharide for capturing the target substance is insoluble and that will not damage the target substance or the insoluble polysaccharide. For target substances that include phytocannabinoids, suitable lipophilic solvents may include alcohol (e.g. methanol, ethanol, n-propyl alcohol, isopropyl alcohol, etc.), other polar organic solvents (e.g. acetone, acetonitrile, tetrahydrofuran, glycerol, DMSO, dichloromethane, chloroform, etc.), eutectic solvents (e.g. equimolar mixture of acetic acid and menthol, glucose syrup, etc.), ionic liquids (e.g. 1-butyl-3-methylimidazolium tetrafluoroborate, etc.), supercritical CO₂ and hydrocarbons (e.g. n-hexane, butane, propane, etc.). The lipophilic solvent may include a suitable combination of any of the above solvents.

The hydrophilic solvent may be any suitable hydrophilic solvent in which a target substance is insoluble or poorly soluble, in which an insoluble polysaccharide for capturing the target substance is insoluble and that will not damage the target substance or the insoluble polysaccharide. The hydrophilic solvent may for example include water, brine, salt solutions or buffered solutions, including solutions comprising a chelating agent.

The lipophilic solvent and the hydrophilic solvent are defined in terms of hydrophobicity and hydrophilicity relative to each other and not necessarily on any particular scale of hydrophobicity and hydrophilicity. For a given lipophilic target compound and a given sample, the lipophilic solvent and the hydrophilic solvent may be selected to be miscible with each other for facilitating recovery of the lipophilic target compound using the insoluble polysaccharide as described above. Where the lipophilic solvent and the hydrophilic solvent are not miscible with each other to any great degree, the lipophilic solvent may be evaporated by increasing heat or by decreasing pressure prior to addition of hydrophilic solvent instead of being mixed with the hydrophilic solvent.

The slurry vessel 20 includes an agitator 22 positioned within the slurry vessel 20. The agitator 22 is for agitating a fluid inside the slurry vessel 20 (e.g. the agitator 22 is shown in FIG. 4 mixing the loaded slurry 52). The agitator 22 is shown as a rotary stirring agitator but any suitable agitator may be used (e.g. cross-flow, a venturi, static agitator, etc.). The slurry vessel 20 is in fluid communication with the filter 12 through a slurry output flow line 24, and fluid communication between the slurry tank 20 and the slurry output flow line 24 may be engaged and disengaged by a output valve 25.

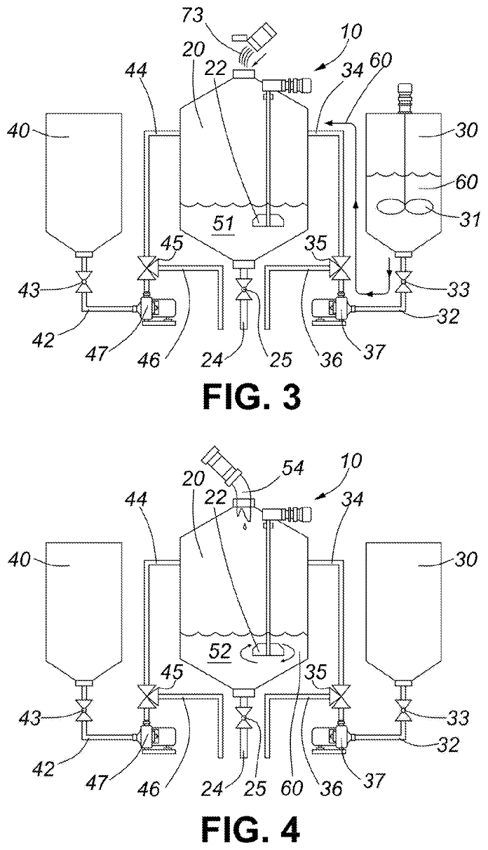

The lipophilic solvent vessel 30 includes an agitator 31 positioned within the lipophilic solvent vessel 30. The agitator 31 is for agitating a lipophilic solvent (e.g. the agitator 31 is shown agitating the lipophilic solvent 60 in FIG. 3, etc.) inside the lipophilic solvent vessel 30 to mix the lipophilic solvent. The lipophilic solvent vessel is in fluid communication with the slurry vessel 20 and with the filter 12.

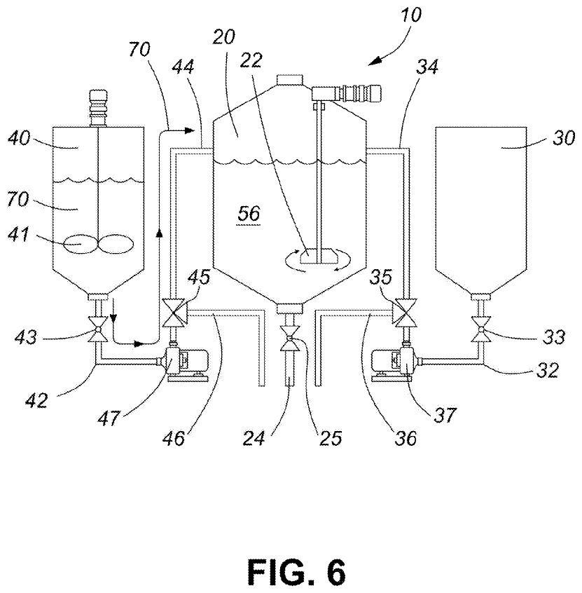

The hydrophilic solvent vessel 40 includes an agitator 41 positioned within the hydrophilic solvent vessel 40. The agitator 41 is for agitating a hydrophilic solvent (e.g. the agitator 41 is shown agitating the hydrophilic solvent 70 in FIG. 6, etc.) inside the hydrophilic solvent vessel 40 to mix the hydrophilic solvent. The hydrophilic solvent vessel 40 is in fluid communication with the slurry vessel 20 and with the filter 12.

The lipophilic solvent vessel 30 may be in fluid communication with the slurry vessel 20 through an upstream lipophilic solvent flow line 32 and a downstream lipophilic solvent flow line 34. Fluid communication between the lipophilic solvent vessel 30 and the slurry vessel 20 may be provided and broken by an upstream lipophilic solvent valve 33 and a downstream lipophilic solvent valve 35. Fluid communication between the lipophilic solvent vessel 30 and the slurry vessel 20 may be driven by a pump 37.

The lipophilic solvent vessel 30 may be in fluid communication with the filter 12 through an upstream lipophilic solvent flow line 32 and a lipophilic solvent rinse flow line 36. Fluid communication between the lipophilic solvent vessel 30 and the filter 12 may be provided and broken by the upstream lipophilic solvent valve 33 and the downstream lipophilic solvent valve 35. Fluid communication between the lipophilic solvent vessel 30 and the filter 12 may be driven by the pump 37.

The hydrophilic solvent vessel 40 may be in fluid communication with the slurry vessel 20 through an upstream hydrophilic solvent flow line 42 and a downstream hydrophilic solvent flow line 44. Fluid communication between the hydrophilic solvent vessel 40 and the slurry vessel 20 may be provided and broken by an upstream hydrophilic solvent valve 43 and a downstream hydrophilic solvent valve 45. Fluid communication between the hydrophilic solvent vessel 40 and the slurry vessel 20 may be driven by a pump 47.

The hydrophilic solvent vessel 40 may be in fluid communication with the filter 12 through an upstream hydrophilic solvent flow line 42 and a hydrophilic solvent rinse flow line 46. Fluid communication between the hydrophilic solvent vessel 40 and the filter 12 may be provided and broken by the upstream hydrophilic solvent valve 43 and the downstream hydrophilic solvent valve 45. Fluid communication between the hydrophilic solvent vessel 40 and the filter 12 may be driven by the pump 47.

Batch Slurry Protocol

FIGS. 3 to 11 show the system 10 in use to purify a lipophilic target compound from a sample 54 using an insoluble adsorbent 73, a lipophilic solvent 60 and a hydrophilic solvent 70. The lipophilic solvent 60 is stored in and sourced from the lipophilic solvent vessel 30. The hydrophilic solvent 70 is stored in and sourced from the hydrophilic solvent vessel 40. For simplicity of review of FIGS. 3 to 11, the lipophilic solvent 60 and the agitator 31 are shown in the lipophilic solvent vessel 30 only when the lipophilic solvent 60 is being supplied to the slurry tank 20. Similarly, and also for simplicity of review of FIGS. 3 to 11, the hydrophilic solvent 70 and the agitator 41 are shown in the hydrophilic solvent vessel 40 only when the hydrophilic solvent 70 is being supplied to the slurry tank 20. In figures where these solvents are not being supplied to the slurry tank 20, the lipophilic solvent vessel 30 and the hydrophilic solvent vessel 40 are shown without detail.

In FIG. 3, the insoluble adsorbent 73, such as a cyclodextrin polymer, is provided into the slurry vessel 20. The insoluble adsorbent 73 may be supplied dry, for example as a powder, and the slurry vessel 20 may be chilled prior to addition of the insoluble adsorbent 73.

The insoluble adsorbent 73 is combined with the lipophilic solvent 60 in the slurry vessel 20 to provide a slurry 51. The lipophilic solvent 60 may be provided to the slurry vessel 20 from the lipophilic solvent vessel 30 via the upstream lipophilic solvent flow line 32 and the downstream lipophilic solvent flow line 34. The lipophilic solvent 60 may be provided in a ratio of 75% insoluble adsorbent 73 to 25% lipophilic solvent 60. Alternatively, either a portion of the insoluble adsorbent 73 or all of the insoluble adsorbent 73 may be added to the slurry vessel 20 after adding the hydrophilic solvent 70 to the slurry vessel 20. Depending on the adsorbent 73 and the hydrophilic solvent 70 that are used, ratios of adsorbent 73: lipophilic solvent 60 may range from 10:90, 9:91, 8:92, 7:93, 6:94, 5:95, 4:96, 3:97, 2:98 or 1:99.

FIG. 4 shows the sample 54 being loaded into the slurry vessel 20 and combined with the slurry 51, providing a loaded slurry 52. The slurry vessel 20 may be chilled to between 3° C. and room temperature, such as 4° C., when the sample 54 is added to the slurry vessel 20. In some cases, lower temperatures may also facilitate maintaining liquidity of a low boiling gaseous solvent, such as butane or other shorter hydrocarbon solvents with boiling points below or close to 20° C. In some cases, lower temperatures may also improve the stability of temperature-sensitive lipophilic target compounds. In some cases, higher temperatures may be applied to decrease solvent viscosity. In some cases, higher temperatures may be used to facilitate in situ decarboxylation of phytocannabinoids, if decarboxylated phytocannabinoids are the target molecule and where decarboxylation was not previous carried out on the sample 54. Temperature may also be modulated to maintain a temperature range at which supercritical fluids have the appropriate physical properties.

The sample 54 includes at least one lipophilic target compound. The sample 54 may include for example an extract or other sample from a biological source (e.g. a plant, animal tissue fungi, yeast, bacteria, or other microorganism), mineral samples (e.g. gold salts, gold complexes, copper salts, copper complexes, etc.), chemical waste samples (e.g. hydrocarbon extraction and processing effluent, mining tailings, etc.). The lipophilic target compound may include any compound that complexes with, binds with or otherwise adheres to the insoluble adsorbent 73. The lipophilic target compound may adhere with the insoluble adsorbent 73 by coordinating within a torus formed by the molecular structure of the insoluble adsorbent 73, or by binding with the insoluble adsorbent 73 outside of the torus.

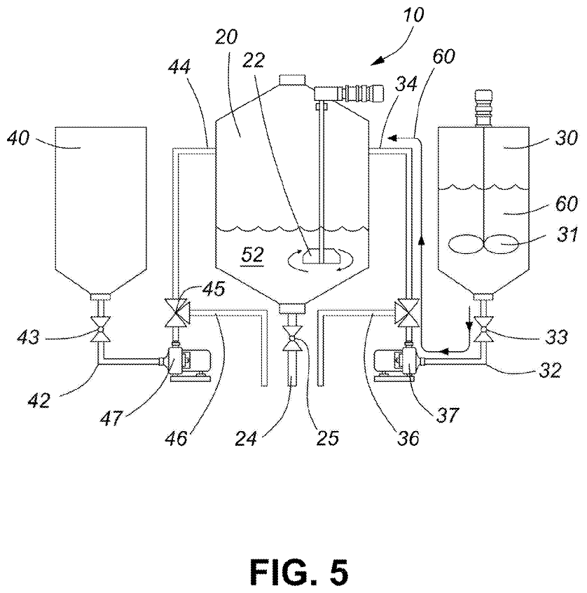

FIG. 5 shows additional lipophilic solvent 60 being added to the slurry vessel 20 to combine with the loaded slurry 52 via the upstream lipophilic solvent flow line 32 and the downstream lipophilic solvent flow line 34. The additional lipophilic solvent 60 may dilute any water that may have been included in the sample 54. The additional lipophilic solvent 60 may facilitate dissolution of phytocannabinoids or other lipophilic target compounds that may be present in the sample 54. The loaded slurry 52 may be agitated by the agitator 22.

FIG. 6 shows the hydrophilic solvent 70 being added to the slurry tank 20 from the lipophilic solvent vessel 30. The hydrophilic solvent 70 may be added to the slurry vessel 20 via the upstream hydrophilic solvent flow line 42 and the downstream hydrophilic solvent flow line 44 and combined with the loaded slurry 52 to provide a binding slurry 56. Where the lipophilic target compound are phytocannabinoids, the sample 54 may be an ethanolic extract of C. sativa flowers or other trichome-bearing biomass, the lipophilic solvent 60 is ethanol and the hydrophilic solvent 70 may be water, the binding slurry 56 may target a ratio of 30:70 lipophilic solvent 60 to hydrophilic solvent 70 for driving the lipophilic target compounds into the insoluble adsorbent 73 polymer core. Other ratios of lipophilic solvent 60 to hydrophilic solvent 70 for the binding slurry 56 may be selected for other lipophilic solvents 60, hydrophilic solvents 70, samples 54 or target lipophilic compounds. Together, the lipophilic solvent 60 and the hydrophilic solvent 70 in a ratio that pushes the target lipophilic target substance into the insoluble adsorbent 73 provide a binding solvent 58. The binding solvent 58 may include miscible lipophilic solvent 60 and hydrophilic solvent 70 or immiscible lipophilic solvent 60 and hydrophilic solvent 70 separated into two layers. Ratios of lipophilic solvent 60:hydrophilic solvent 70 may range from 95:5, 90:10, 85:15, 80:20, 75:25, 70:30, 65:35, 60:40, 55:45, 50:50, 45:55, 40:60, 35:65, 30:70, 25:75, 20:80, 15:85, 10:90 and 5:95.

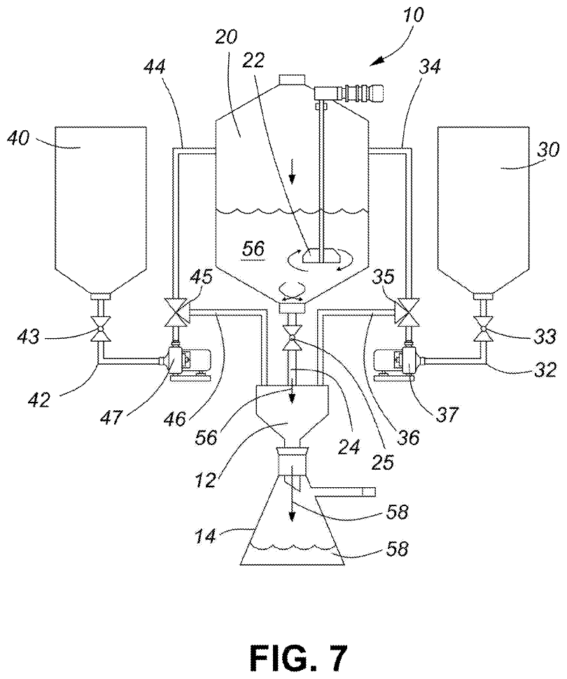

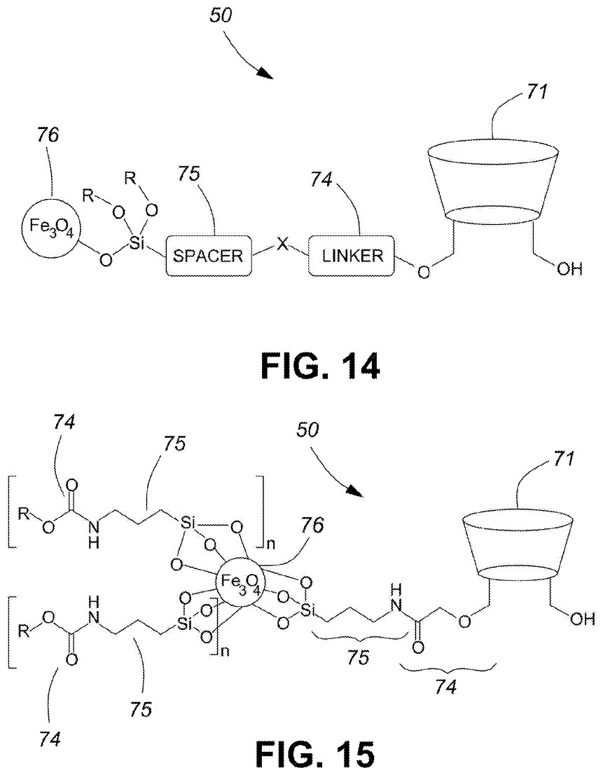

FIG. 7 shows the binding slurry 56 being run through the filter 12 for filtering and retaining the insoluble adsorbent 73 with captured lipophilic target compounds. The binding solvent 58 runs through the filter 12 into the recovery vessel 14. The filter 12 may comprise paramagnetic or other magnetic qualities for magnetically attracting or retaining embodiments of the insoluble adsorbent 73 bound to a magnetic particle or a magnetic nanoparticle on the filter 12, such as the embodiments of the insoluble adsorbent 73 shown in FIGS. 14 to 17.

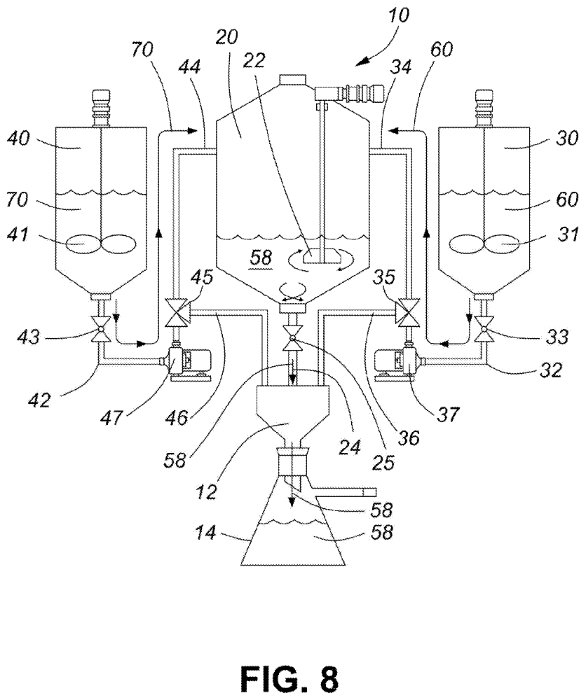

FIG. 8 shows rinsing of the filter 12 with the binding solvent 58 or other ratios of the lipophilic solvent 60 and the hydrophilic solvent 70 to wash the filter 12. Rinsing with the binding solvent 58 may remove some material (e.g. chlorophyll, CBDA, etc.) that water by itself may not remove. This step may also recover some valuable material that binds less strongly than a target hydrophobic material, such as recovery of CBDA when decarboxylated CBD is the primary lipophilic target compound. Such valuable material may be repurified through the system 10. Providing the binding solvent 58 to the filter 12 through the downstream lipophilic solvent flow line 34 and the downstream hydrophilic solvent flow line 44 may rinse out the slurry tank 20. The binding solvent 58 may be provided to the filter 12 by direct application of the lipophilic solvent 60 and the hydrophilic solvent 70 to the filter 12 through the lipophilic solvent rinse flow line 36 and the hydrophilic solvent rinse flow line 46.

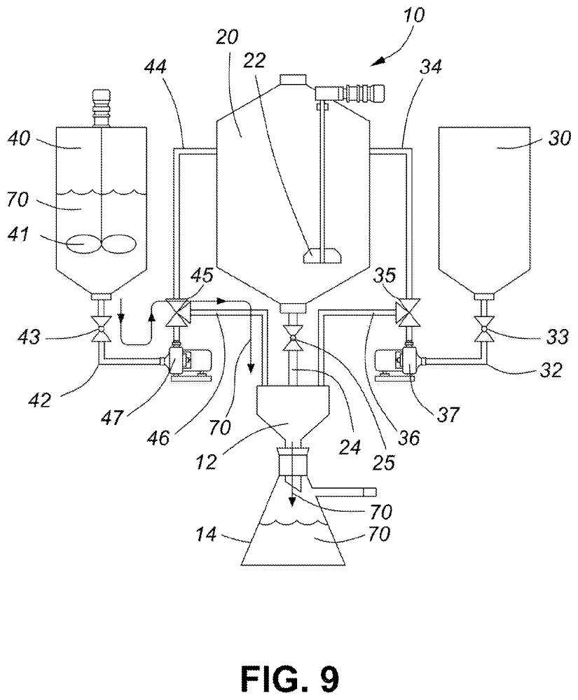

FIG. 9 shows rinsing of the filter 12 with hydrophilic solvent 70 to wash the filter 12 via the upstream hydrophilic solvent flow line 42 and the hydrophilic solvent rinse flow line 46. An amount of hydrophilic solvent 70 used to wash the filter 12 may be about 3 or 4 times the volume of the binding slurry 56 that was passed through the filter 12.

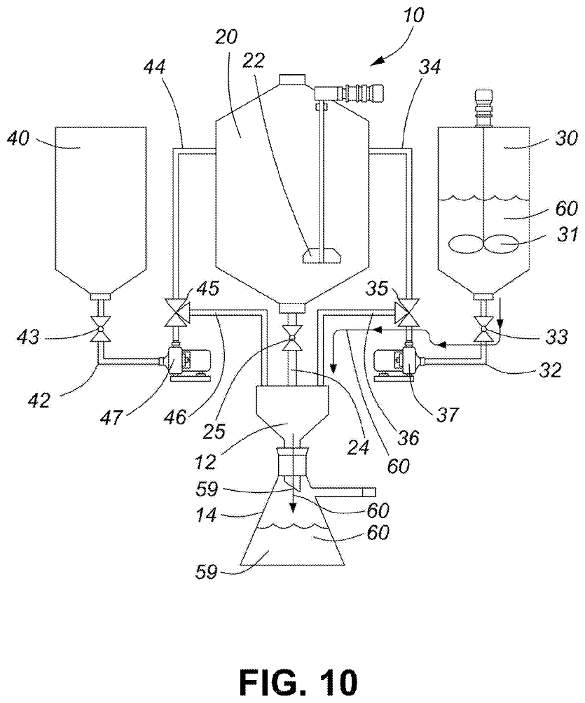

FIG. 10 shows dissolution of the lipophilic target compounds by flowing the lipophilic solvent 60 over the filter 12 to dissociate the lipophilic target compounds from the insoluble adsorbent 73 and solubilize the lipophilic target compounds in the lipophilic solvent 60. A recovered lipophilic target compound 59 is recovered in the lipophilic solvent 60 from the recovery vessel 14 The amount of lipophilic solvent 60 used to recover the recovered lipophilic target compound 59 may be selected to provide the recovered lipophilic target compound 59 at a defined concentration. A lipophilic solvent other than the lipophilic solvent 60 may be used to recover the recovered lipophilic target compound 59.

The insoluble adsorbent 73 may then be regenerated for reuse by washing the insoluble adsorbent 73 with a detergent solution, for example 0.1% Triton X-100 at 37° C. for one minute. Solvents that are able to dissociate any lipophilic compounds from the insoluble adsorbent 73, such as DMSO, may also be applied for regeneration. Exposure to the detergent solution, to solvent or other regeneration may be followed by re-equilibration with 3 to 5 volumes of ethanol.

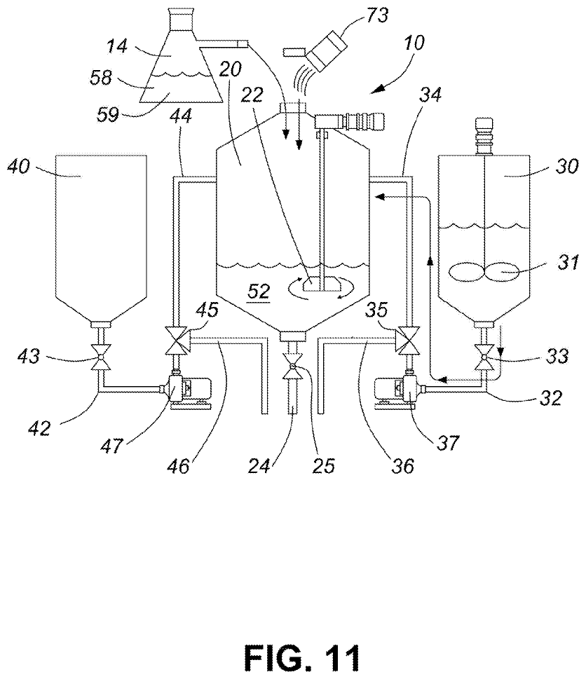

FIG. 11 shows that the contents of the recovery vessel 14 after washing of the ethanol extract may then be loaded into the chilled slurry vessel 20 to repeat the batch slurry protocol with an aliquot of unique cyclodextrin polymer (for example a-cyclodextrin or γ-cyclodextrin).

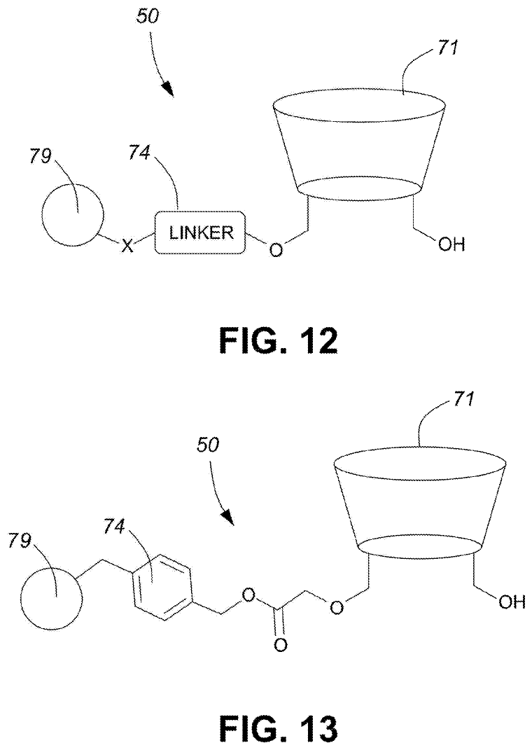

FIG. 12 shows an embodiment of the insoluble adsorbent 73 in which a polysaccharide 71 bound to an insoluble polymer 79, such as an insoluble polymeric bead (e.g. a polystyrene bead, Merrifield polystyrene resin bead, Wang resin bead, etc.). The polysaccharide 71 is bound to the insoluble polymer 79 by a linker 74.

FIG. 13 shows an embodiment of the insoluble adsorbent 73 in which the polysaccharide 71 is bound to the insoluble polymer 79 by the linker 74, and the linker 74 comprises a benzylic ester, in this case a carboxymethylene group.

FIG. 14 shows an embodiment of the insoluble adsorbent 73 in which the polysaccharide 71 is bound to a magnetic bead 76 by the linker 74, and by a spacer 75, which may include a silicate group. The magnetic bead 76 may include a micron-sized magnetite particle or other magnetic material.

FIG. 15 shows an embodiment of the insoluble adsorbent 73 in which the polysaccharide 71 is bound to the magnetic bead 76 by the linker 74, which comprises an amide group, and by the spacer 75, which comprises a propyl group. In FIG. 15, multiple separate spacer groups 75 are bound with the magnetic bead 76 to coordinate multiple insoluble polysaccharides 50 with the magnetic bead 76.

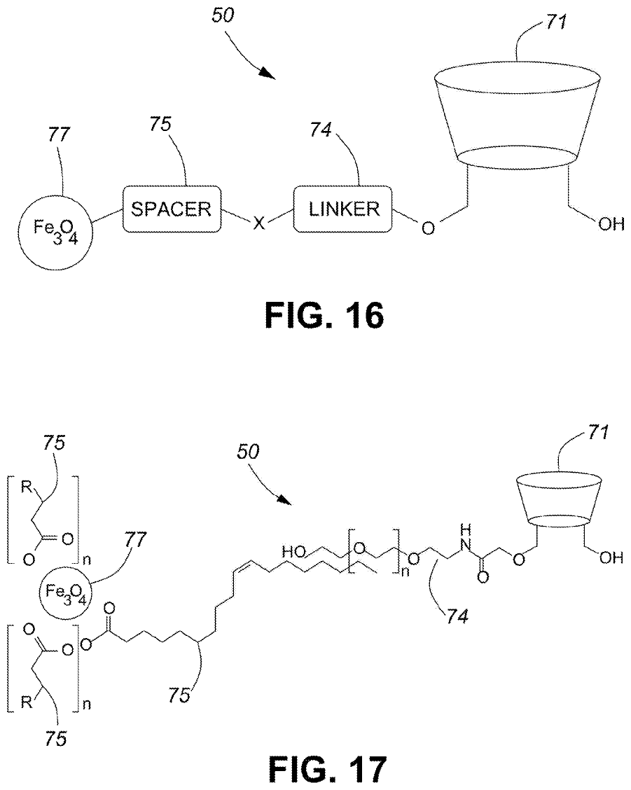

FIG. 16 shows an embodiment of the insoluble adsorbent 73 in which the polysaccharide 71 is bound to a magnetic nanoparticle 77 by the linker 74 and the spacer 75. The magnetic nanoparticle 77, may include a micron-sized magnetite particle or other magnetic material.

FIG. 17 shows an embodiment of the insoluble adsorbent 73 in which the polysaccharide 71 is bound to the magnetic nanoparticle 77 by the linker 74 and the spacer 75. The linker 74 includes a polyethylene glycol linker and amide, which binds non-covalently through Van der Waals hydrophobic interaction with the spacer 75, which includes a monounsaturated hydrocarbon carboxylate. In FIG. 17, multiple separate spacer groups 75 are bound with the magnetic or magnetic nanoparticle 77 to coordinate multiple insoluble polysaccharides 50 with the magnetic nanoparticle 77.

Embodiments of the insoluble adsorbent 73 shown in FIGS. 12 to 17 may be used in combination, with different filters 12 or other isolation methods being used to target different embodiments of the insoluble adsorbent 73. For example, the embodiments of the insoluble adsorbent 73 shown in FIGS. 12 and 13 could be recovered with a filter 12 sized for the particular insoluble polymer 79 used, while at the same time the embodiments of the insoluble adsorbent 73 shown in FIGS. 14 to 17 could be recovered by application with a magnetic field to the binding slurry 56. The magnetic field could be applied to the binding slurry by using a filter 12 that includes a magnetron or other source of a magnetic field, by immersing a magnetron or other source of a magnetic field in the binding slurry 56 or any suitable method of exposing a magnetic field to the binding slurry such that the magnetic bead 76, or magnetic nanoparticle 77, is drawn toward the magnetic field. If each polysaccharide 71 has a preferred propensity for binding different lipophilic target substances, then multiple insoluble polysaccharides 50 of FIGS. 12 to 17 could be used in combination on a given sample 54 and then easily separated, separating different recovered lipophilic target substances 59 from the same binding slurry 56.

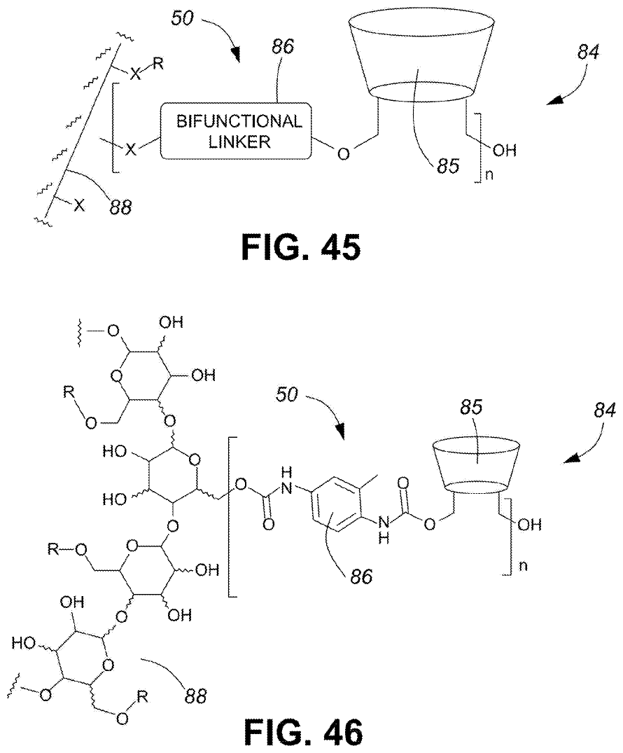

In addition to the embodiments of the insoluble adsorbent 73 shown in FIGS. 12 to 17, the embodiments of the insoluble adsorbent 73 shown in FIGS. 45 to 48 may also be used in the system 10 where the immobile matrix 88 or the silica-based immobile matrix 89 are reduced in size to allow the embodiments of the insoluble adsorbent 73 shown in FIGS. 45 to 48 to be used in a slurry rather than as part of an immobile phase in a column or other chromatographic separation technique.

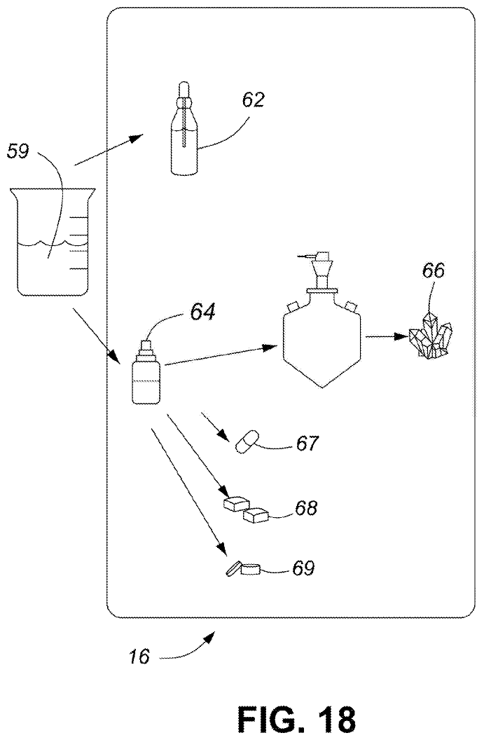

FIG. 18 shows the recovered target compound 59 recovered from the filter 12 may be provided to the processing system 16 for processing into downstream products for sale to consumers or in business to business transactions. Considering an application where the recovered target compound 59 is from hemp or other C. sativa extract, the recovered target compound 59 may be as an input for a tincture 62 (e.g. an ethanol tincture, food oil tincture, etc.). The recovered target compound 59 from hemp or other C. sativa extract may be processed with supercritical CO₂ or other extraction and formulation to produce a full spectrum extract oil 64 for oil-based products. The full spectrum extract oil 64 may then be further processed for specific phytocannabinoids and crystallized to produce an isolate 66. Alternatively, the full spectrum extract oil may be used to produce products such as capsules 67, edibles 68 or salves 69.

Column Capture Setup

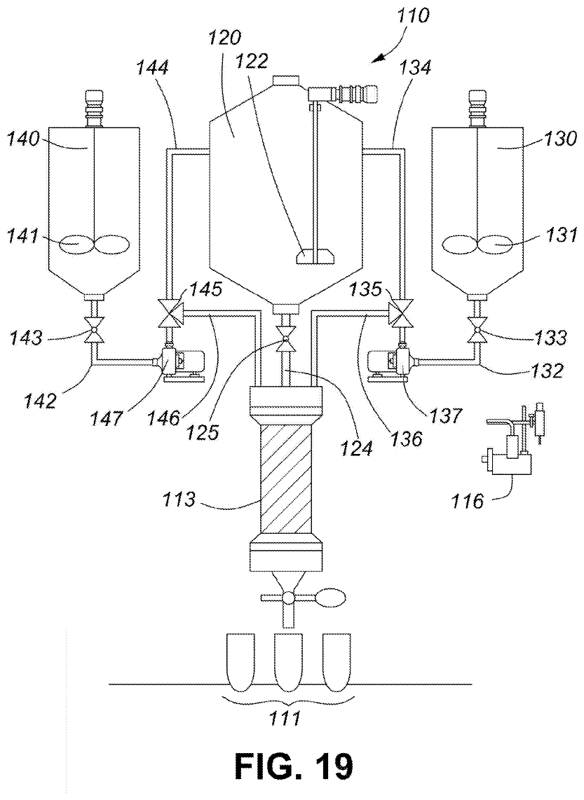

FIG. 19 shows a lipophilic compound recovery system 110. The system 110 includes the slurry vessel 120. A column filter 113 is in fluid communication with the slurry vessel 120 for receiving fluid from the slurry vessel 120 and filtering material out of the fluid. A recovery vessel 111 is in fluid communication with the column filter 113 for receiving filtrate that passes through the column filter 113. In the system 110, a series of individual recovery vessels 111 are applied for selective elution from the column filter 113, but a single recovery vessel 111 may be applied. Each recovery vessel 111 may be any suitable recovery vessel may be used (e.g. a flask, Erlenmeyer, round-bottom flask, beaker, test tube, etc.). The processing system 116 may be in fluid communication with the recovery vessel 111 for processing target molecules captured using the column filter 113. The slurry vessel 120 is in fluid communication with the lipophilic solvent vessel 130 for receiving lipophilic solvent from the lipophilic solvent vessel 130. The slurry vessel 120 is in fluid communication with the hydrophilic solvent vessel 140 for receiving hydrophilic solvent from the hydrophilic solvent vessel 140.

Each of the slurry vessel 120, the lipophilic solvent vessel 130 and the hydrophilic solvent vessel 140 may be any suitable fluid vessel appropriate for the size, scale and application of the system 110 (e.g. a tank, pressure-rated tank, beaker, etc.).

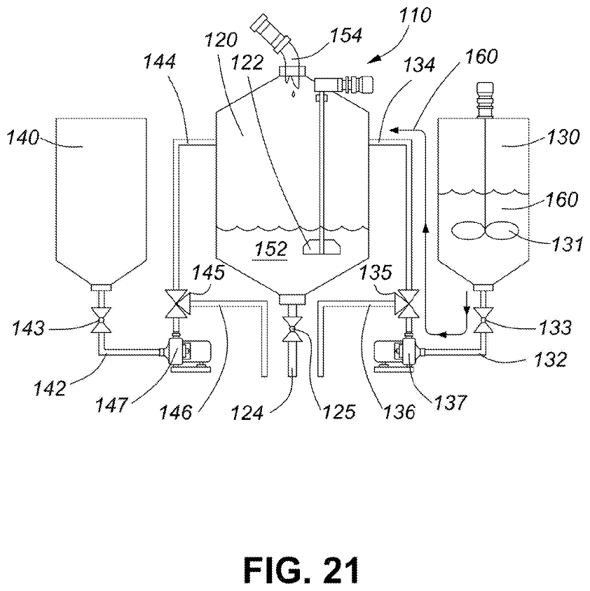

The slurry vessel 120 includes the agitator 122 positioned within the slurry vessel 120. The agitator 122 is for agitating a fluid inside the slurry vessel 120 (e.g. the agitator 122 is shown in FIG. 21 mixing the loaded slurry 152). The agitator 122 is shown as a rotary stirring agitator but any suitable agitator may be used (e.g. cross-flow, a venturi, static agitator, etc.). The slurry vessel 120 is in fluid communication with the column filter 113 through the slurry output flow line 124, and fluid communication between the slurry tank 120 and the slurry output flow line 124 may be engaged and disengaged by the output valve 125.

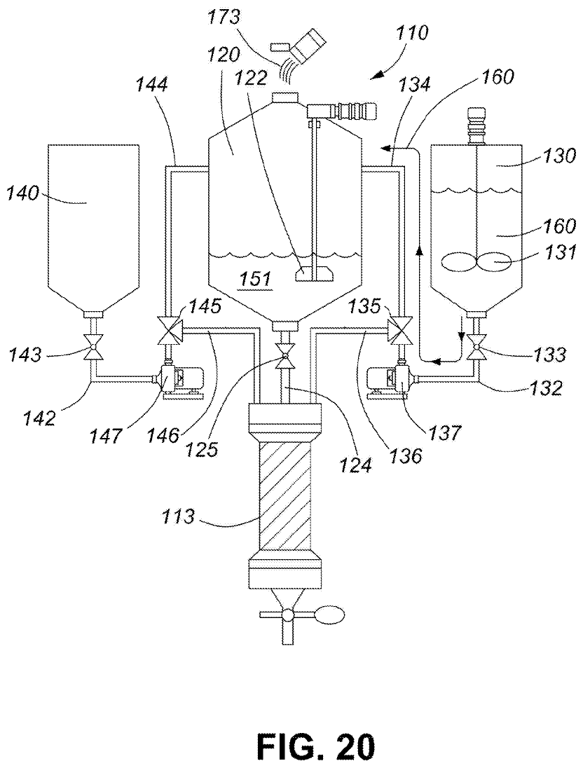

The lipophilic solvent vessel 130 includes the agitator 131 positioned within the lipophilic solvent vessel 130. The agitator 131 is for agitating a lipophilic solvent (e.g. the agitator 131 is shown agitating the lipophilic solvent 160 in FIG. 20, etc.) inside the lipophilic solvent vessel 130 to mix the lipophilic solvent. The lipophilic solvent vessel 130 is in fluid communication with the slurry vessel 120 and with the column filter 113.

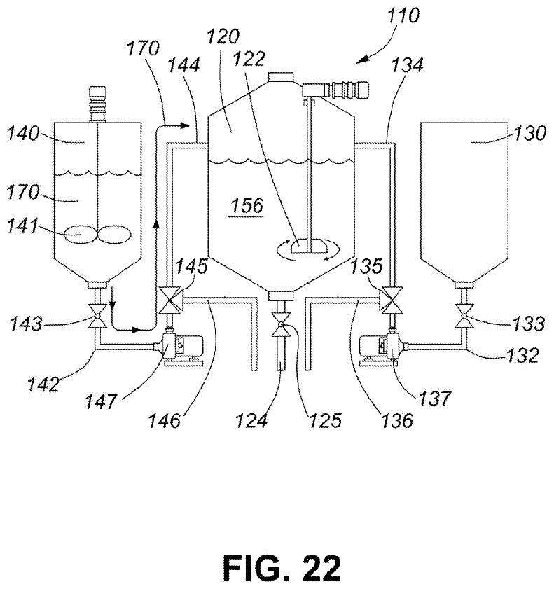

The hydrophilic solvent vessel 140 includes the agitator 141 positioned within the hydrophilic solvent vessel 140. The agitator 141 is for agitating a hydrophilic solvent (e.g. the agitator 141 is shown agitating the hydrophilic solvent 170 in FIG. 22, etc.) inside the hydrophilic solvent vessel 140 to mix the hydrophilic solvent. The hydrophilic solvent vessel 140 is in fluid communication with the slurry vessel 120 and with the column filter 113.

The lipophilic solvent vessel 130 may be in fluid communication with the slurry vessel 120 through the upstream lipophilic solvent flow line 132 and the downstream lipophilic solvent flow line 134. Fluid communication between the lipophilic solvent vessel 130 and the slurry vessel 120 may be provided and broken by the upstream lipophilic solvent valve 133 and the downstream lipophilic solvent valve 135. Fluid communication between the lipophilic solvent vessel 130 and the slurry vessel 120 may be driven by the pump 137.

The lipophilic solvent vessel 130 may be in fluid communication with the column filter 113 through the upstream lipophilic solvent flow line 132 and the lipophilic solvent rinse flow line 136. Fluid communication between the lipophilic solvent vessel 130 and the column filter 113 may be provided and broken by the upstream lipophilic solvent valve 133 and the downstream lipophilic solvent valve 135. Fluid communication between the lipophilic solvent vessel 130 and the column filter 113 may be driven by the pump 137.

The hydrophilic solvent vessel 140 may be in fluid communication with the slurry vessel 120 through the upstream hydrophilic solvent flow line 142 and the downstream hydrophilic solvent flow line 144. Fluid communication between the hydrophilic solvent vessel 140 and the slurry vessel 120 may be provided and broken by the upstream hydrophilic solvent valve 143 and the downstream hydrophilic solvent valve 145. Fluid communication between the hydrophilic solvent vessel 140 and the slurry vessel 120 may be driven by the pump 147.

The hydrophilic solvent vessel 140 may be in fluid communication with the column filter 113 through the upstream hydrophilic solvent flow line 142 and the hydrophilic solvent rinse flow line 146. Fluid communication between the hydrophilic solvent vessel 140 and the column filter 113 may be provided and broken by the upstream hydrophilic solvent valve 143 and the downstream hydrophilic solvent valve 145. Fluid communication between the hydrophilic solvent vessel 140 and the column filter 113 may be driven by the pump 147.

Column Capture Protocol

FIGS. 19 to 28 show the system 110 in use to purify a lipophilic target compound using insoluble polysaccharides. The lipophilic solvent 160 is stored in and sourced from the lipophilic solvent vessel 130. The hydrophilic solvent 170 is stored in and sourced from the hydrophilic solvent vessel 140. For simplicity of review of FIGS. 19 to 28, the lipophilic solvent 160 and the agitator 131 are shown in the lipophilic solvent vessel 130 only when the lipophilic solvent 160 is being supplied to the slurry tank 120. Similarly, and also for simplicity of review of FIGS. 19 to 28, the hydrophilic solvent 170 and the agitator 141 are shown in the hydrophilic solvent vessel 140 only when the hydrophilic solvent 170 is being supplied to the slurry tank 120. In figures where these solvents are not being supplied to the slurry tank 120, the lipophilic solvent vessel 130 and the hydrophilic solvent vessel 140 are shown without detail.

In FIG. 20, the insoluble adsorbent 173, such as a cyclodextrin polymer, is provided into the slurry vessel 120. The insoluble adsorbent 173 may be supplied dry, for example as a powder, and the slurry vessel 120 may be chilled prior to addition of the insoluble adsorbent 173. The insoluble adsorbent 173 is combined with the lipophilic solvent 160 in the slurry vessel 120 to provide the slurry 151. The lipophilic solvent 160 may be provided to the slurry vessel 120 from the lipophilic solvent vessel 130 via the upstream lipophilic solvent flow line 132 and the downstream lipophilic solvent flow line 134. The lipophilic solvent 160 may be provided in a ratio of 75% insoluble adsorbent 73 to 25% lipophilic solvent 160.

FIG. 21 shows the sample 154 being loaded into the slurry vessel 120 and combined with the slurry 151, providing the loaded slurry 152. The sample 154 includes at least one lipophilic target compound.

FIG. 22 shows the hydrophilic solvent 170 being added to the slurry vessel 120 from the lipophilic solvent vessel 130. The hydrophilic solvent 170 may be added to the slurry vessel 120 via the upstream hydrophilic solvent flow line 142 and the downstream hydrophilic solvent flow line 144 and combined with the loaded slurry 152 to provide the binding slurry 156. The binding slurry 156 may include the binding solvent 158 with a ratio of lipophilic solvent 160 to hydrophilic solvent 170 selected to drive the lipophilic target compounds into the insoluble adsorbent 173 polymer core or otherwise bind with the insoluble adsorbent 173.

A binding solvent 158, which may have a ratio of lipophilic solvent 160 to hydrophilic solvent 170 similar to the ratio targeted in the binding slurry 156, may be added to an insoluble polysaccharide in to provide a stationary phase solution. The insoluble polysaccharide solution may be poured into the column filter 113 having a glass fibre frit to pack the column filter 113.

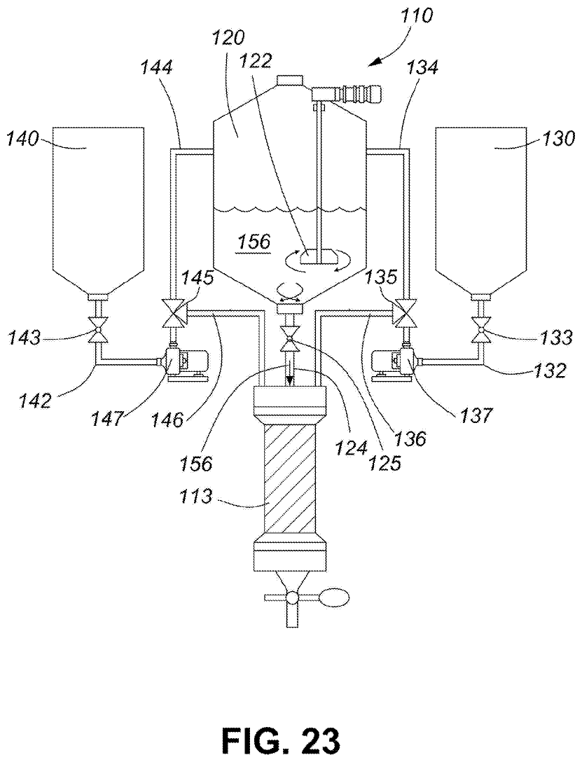

FIG. 23 shows the binding slurry 156 being emptied into the column filter 113 for loading the insoluble adsorbent 173 with captured lipophilic target compounds onto the pre-wetted insoluble polysaccharide stationary phase in the column filter 113, and the lipophilic target substances may adsorb onto the stationary phase of the column filter 113. Once loaded, load permeate may be collected for storage in a flow-through reservoir (not shown; similar to the flow-through vessels 280 or 380).

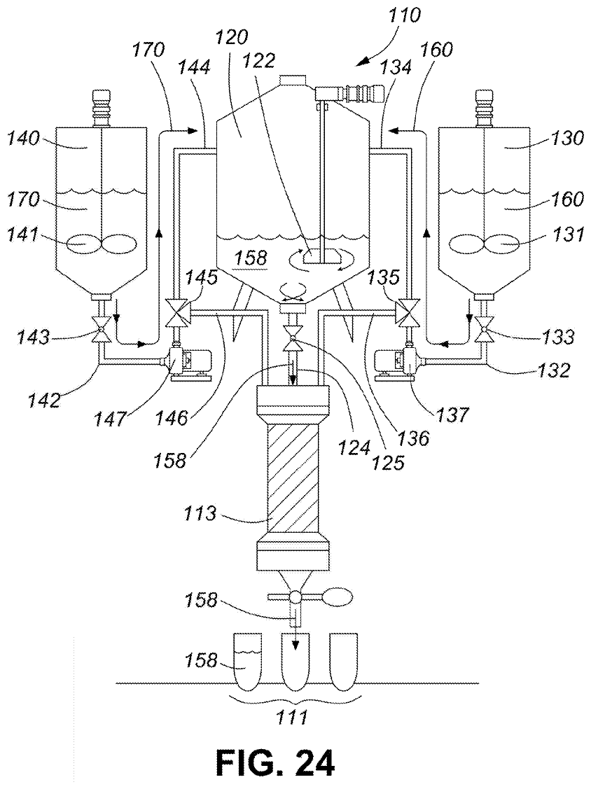

FIG. 24 shows the loaded column filter 113 being washed with the binding solvent 158 in the slurry vessel 120. The binding solvent 158 may comprise lipophilic solvent 160 and hydrophilic solvent 170 in the same target ratio as used in the binding slurry 156. The binding solvent 158 passes through the column filter 113 and into the recovery vessels 111. If any insoluble adsorbent 173 or passes through the column filter 113 with lipophilic target substances bound to the insoluble polysaccharide, the insoluble adsorbent 173 and lipophilic target substances maybe recovered from the recovery vessels 111.

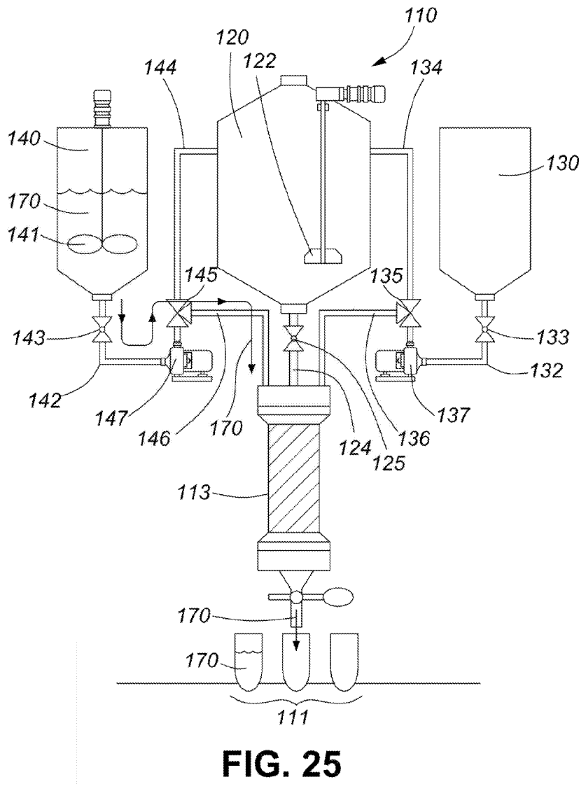

FIG. 25 shows rinsing of the column filter 113 with hydrophilic solvent 170 to wash the column filter 113 via the upstream hydrophilic solvent flow line 142 and the hydrophilic solvent rinse flow line 146. An amount of the hydrophilic solvent 170 equal to three or four times the volume of the binding slurry 116 may be passed through the column filter 113 to wash the stationary phase with bound lipophilic target substances. The hydrophilic solvent 170 may be collected in the recovery vessels 111.

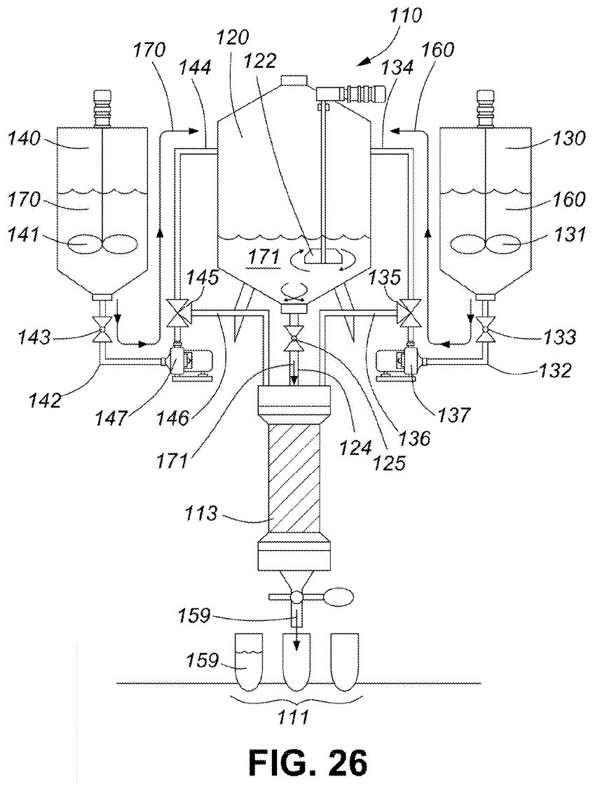

FIG. 26 shows rinsing of the column filter 113 with binding solvent 158, or with other mixtures of the lipophilic solvent 160 and the hydrophilic solvent 170 to wash the column filter 113 after mixing the lipophilic solvent 160 and the hydrophilic solvent 170 in the slurry vessel 120. The amount of the lipophilic solvent 160 included in the mixture of the lipophilic solvent 160 and the hydrophilic solvent 170 may be increased over time to elute progressively more tightly bound lipophilic target compounds, providing the recovered lipophilic target compounds 159. The recovered lipophilic target compounds 159 may be collected in the recovery vessels 111.

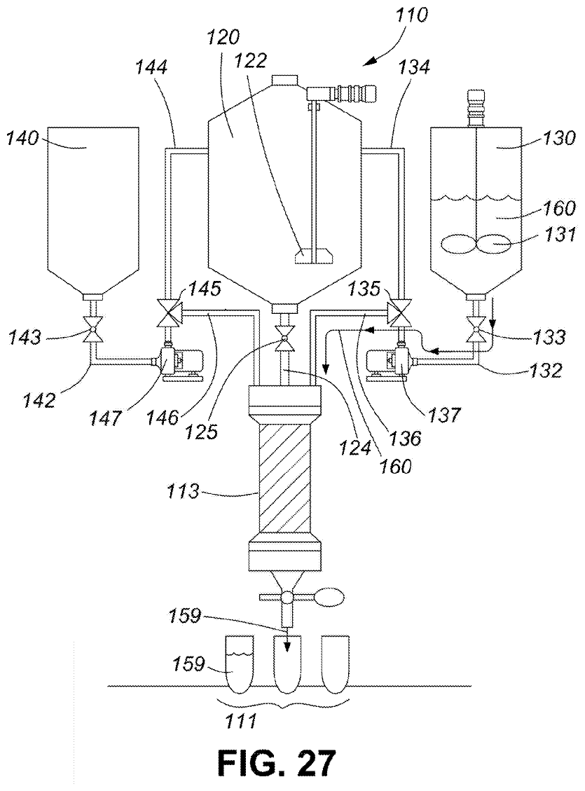

FIG. 27 shows the loaded column filter 113 being eluted with the lipophilic solvent 160 for dissociating the lipophilic target compounds from the stationary phase comprising the insoluble adsorbent 173 and for solubilizing the lipophilic target compounds in the lipophilic solvent 160. The column filter 113 is eluted until no more of the lipophilic target compound is eluted and the output of lipophilic target compounds is stable. The recovered lipophilic target compounds 159 may be collected in the recovery vessels 111.

The insoluble adsorbent 173 may then be regenerated for reuse by washing the insoluble adsorbent 173 with a detergent solution, for example 0.1% Triton X-100 at 37° C. for one minute. Solvents that are able to dissociate any lipophilic compounds from the insoluble adsorbent 173, such as DMSO, may also be applied for regeneration. Exposure to the detergent solution, to solvent or other regeneration may be followed by re-equilibration with 3 to 5 volumes of ethanol.

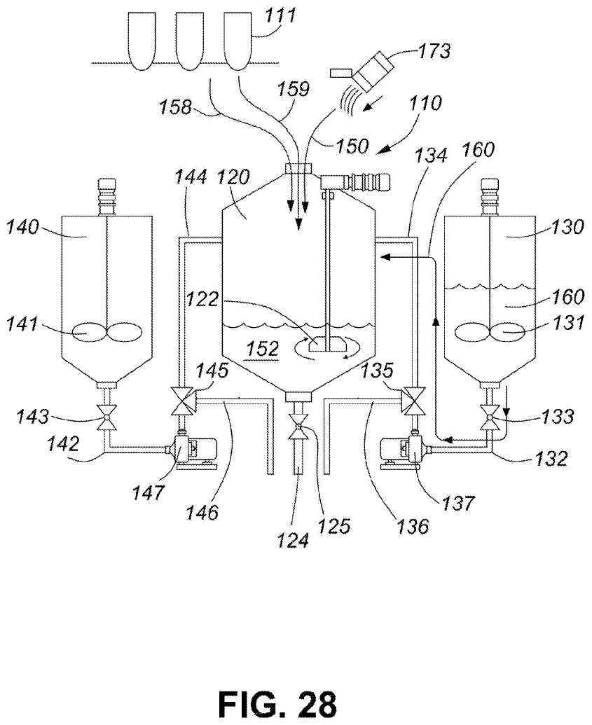

FIG. 28 shows that permeate that may have been previously collected in recovery vessel 111 may be loaded into the slurry vessel 120 to repeat the process shown in FIGS. 23 to 27. Where the permeate is passed through the system 110 again, the insoluble adsorbent 173 used the second time may be different than the insoluble adsorbent 173 initially used, for example an α-cyclodextrin or γ-cyclodextrin may be used after a β-cyclodextrin.

Immersion Filter Capture Setup

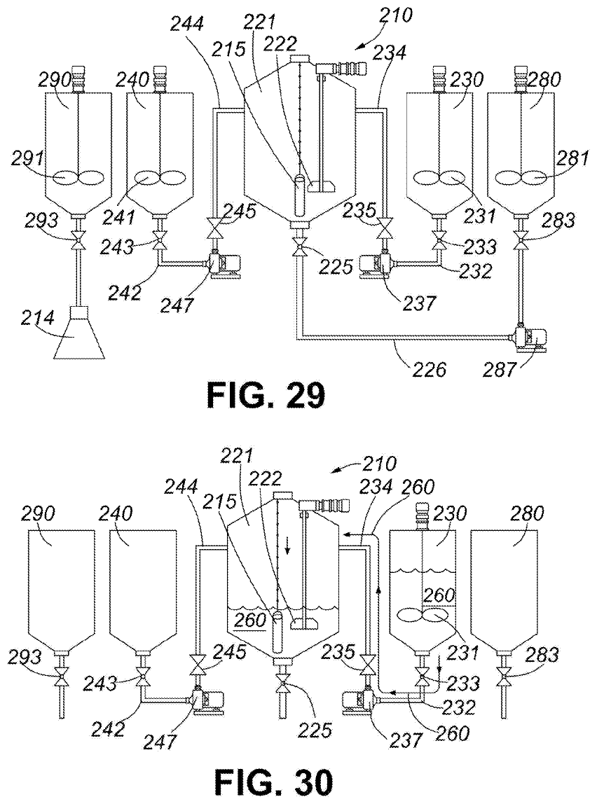

FIG. 29 shows a lipophilic compound recovery system 210. The system 210 includes a binding vessel 221. An immersion filter, for example, an immersion filter 215 is sized to be housed inside the binding vessel 221 for being in fluid communication with the binding vessel 221, receiving fluid from the binding vessel 221 and filtering material out of the fluid. The immersion filter 215 houses the insoluble polysaccharide 250. The immersion filter 215 may include insoluble polysaccharide bound with a matrix attached to the immersion filter 215, such as the embodiments of the insoluble adsorbent 73 shown in FIGS. 45 to 48. The immersion filter 215 may include insoluble polysaccharide sequestered within the immersion filter 215 by a pore size smaller than an insoluble polymer 79, magnetic bead 76, magnetic nanoparticle 77 or other insoluble material bound to, complexed with or otherwise adhered to the polysaccharide 71 included in the insoluble adsorbent 73, such as the embodiments of the insoluble adsorbent 73 shown in FIGS. 12 to 17. The insoluble adsorbent

The immersion filter 215 is sized to receive filtrate that passes through the immersion filter 215. The immersion filter 215 may be a mesh sized to prevent pass through of an adsorbent inside the immersion filter 215. The immersion filter 215 may be a material that does not bind to the target substance, or the adsorbent may be bound to the immersion filter 215. The immersion filter 215 may include a woven mesh membrane in the form of a pouch which may comprise medical-grade polyester or nylon, or other materials that will not bind the lipophilic target substances to any significant degree. Commercially-available membranes that may be applied in the immersion filter 215 may include Supor® 200, 800, 1200 hydrophilic polyethylene sulfonate (“PES”) membranes (Gelman Sciences (Ann Arbor, Mich.)); Durapore® hydrophilic modified polyvinylidene difluoride (“PVDF”) (Mantee America Corp. (San Diego, Calif.)) and hydrophilic modified polysulfone membranes with integrated hydrophobic vents, for example, Gemini membranes, (Millipore (Marlborough, Mass.)); and membranes comprising polycarbonate with a polyvinylidene coating (Poretics (Livermore, Calif.)), a stainless steel mesh or a glass frit. The immersion filter 215 can be sterilized after addition of the adsorbent. The mesh material of the immersion filter 215 may be selected such that the intended lipophilic target compound does not bind to the mesh material.

The binding vessel 221 is in fluid communication with the lipophilic solvent vessel 230 for receiving lipophilic solvent from the lipophilic solvent vessel 230. The binding vessel 221 is in fluid communication with the hydrophilic solvent vessel 240 for receiving hydrophilic solvent from the hydrophilic solvent vessel 240. The binding vessel 221 is in fluid communication with a flow-through vessel 280 for storing the binding solution 258 after exposure of the sample 254 to the immersion filter. A wash vessel 290 is in fluid communication with the recovery vessel 214 for receiving waste hydrophilic solvent 270 or binding solvent 258.

Each of the binding vessel 221, the lipophilic solvent vessel 230 and the hydrophilic solvent vessel 240 may be any suitable fluid vessel appropriate for the size, scale and application of the system 210 (e.g. a tank, pressure-rated tank, etc.).

The binding vessel 221 includes the agitator 222 positioned within the binding vessel 221. The agitator 222 is for agitating a fluid inside the binding vessel 221 (e.g. the propeller shown in FIG. 29) to mix the fluid. The agitator 222 is shown as a rotary stirring agitator but any suitable agitator may be used (e.g. cross-flow, a venturi, static agitator, etc.). The binding vessel 221 is in fluid communication with the immersion filter 215 direct contact with the binding solvent 258.

The lipophilic solvent vessel 230 includes the agitator 231 positioned within the lipophilic solvent vessel 230. The agitator 231 is for agitating a lipophilic solvent (e.g. the rotary stirring agitator 231 agitating the lipophilic solvent 260 as shown in FIG. 30) inside the lipophilic solvent vessel 230 to mix the lipophilic solvent.

The lipophilic solvent vessel 230 may be in fluid communication with the binding vessel 221 through the upstream lipophilic solvent flow line 232 and the downstream lipophilic solvent flow line 234. Fluid communication between the lipophilic solvent vessel 230 and the binding vessel 221 may be provided and broken by the upstream lipophilic solvent valve 233 and the downstream lipophilic solvent valve 235. Fluid communication between the lipophilic solvent vessel 230 and the binding vessel 221 may be driven by the pump 237.

The lipophilic solvent vessel 230 may be in fluid communication with the immersion filter 215 through the upstream lipophilic solvent flow line 232 and the downstream hydrophobic flow line 234 when the immersion filter 215 is immersed in the liquid contents of the binding vessel 221, for example, the binding solvent 258. Fluid communication between the lipophilic solvent vessel 230 and the immersion filter 215 may be provided and broken by the upstream lipophilic solvent valve 233 and the downstream lipophilic solvent valve 235 and by contact between the immersion filter 215 and the contents of the binding vessel 221. Fluid communication between the lipophilic solvent vessel 230 and the immersion filter 215 may be driven by the pump 237.

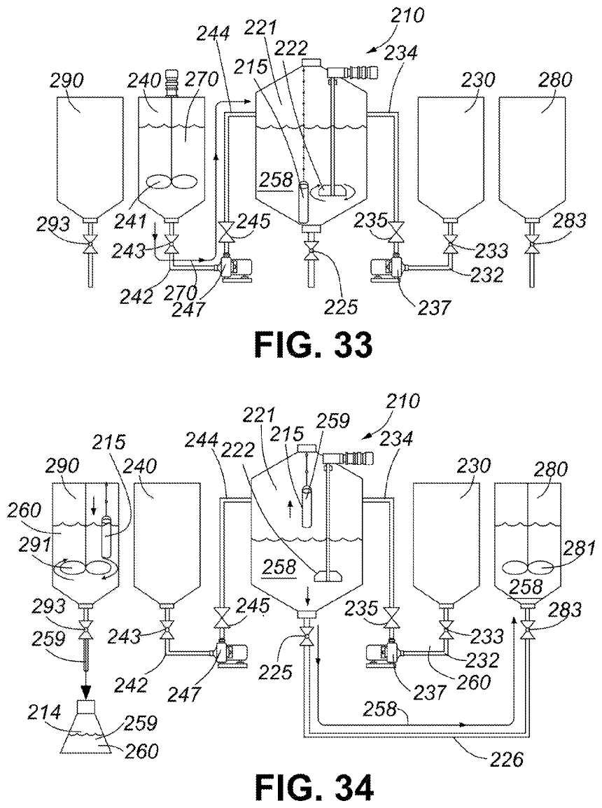

The hydrophilic solvent vessel 240 includes the agitator 241 positioned within the hydrophilic solvent vessel 240. The agitator 241 is for agitating a hydrophilic solvent (e.g. the rotary stirring agitator 241 agitating the hydrophilic solvent 270 as shown in FIG. 33) inside the hydrophilic solvent vessel 240 to mix the hydrophilic solvent 270.

The hydrophilic solvent vessel 240 may be in fluid communication with the binding vessel 221 through the upstream hydrophilic solvent flow line 242 and the downstream hydrophilic solvent flow line 244. Fluid communication between the hydrophilic solvent vessel 240 and the binding vessel 221 may be provided and broken by the upstream hydrophilic solvent valve 243 and the downstream hydrophilic solvent valve 245. Fluid communication between the hydrophilic solvent vessel 240 and the binding vessel 221 may be driven by the pump 247.

The hydrophilic solvent vessel 240 may be in fluid communication with the immersion filter 215 through the upstream hydrophilic solvent flow line 242 and the downstream hydrophilic solvent flow line 244 when the immersion filter 215 is immersed in the liquid contents of the binding vessel 221, for example, the binding solvent 258. Fluid communication between the hydrophilic solvent vessel 240 and the immersion filter 215 may be provided and broken by the upstream hydrophilic solvent valve 243 and the downstream hydrophilic solvent valve 245 and by contact between the immersion filter 215 and the contents of the binding vessel 221. Fluid communication between the hydrophilic solvent vessel 240 and the immersion filter 215 may be driven by the pump 247.

The flow-through vessel 280 may be in fluid communication with the binding vessel 221 through a flow-through line 226. Fluid communication between the flow-through vessel 280 and the binding vessel 221 may be provided and broken by the output valve 225 and a flow-through valve 283. Fluid communication between the flow-through vessel 280 and the binding vessel 221 may be driven by a pump 287.

The flow-through vessel 280 may be in fluid communication with the immersion filter 215 through the upstream lipophilic solvent flow line 226 when the immersion filter 215 is immersed in the liquid contents of the binding vessel 221, for example, the binding solvent 258. Fluid communication between the flow-through vessel 226 and the immersion filter 215 may be provided and broken by the flow-through valve 283 and by contact between the immersion filter 215 and the contents of the binding vessel 221. Fluid communication between the flow-through vessel 280 and the immersion filter 215 may be driven by the pump 287.

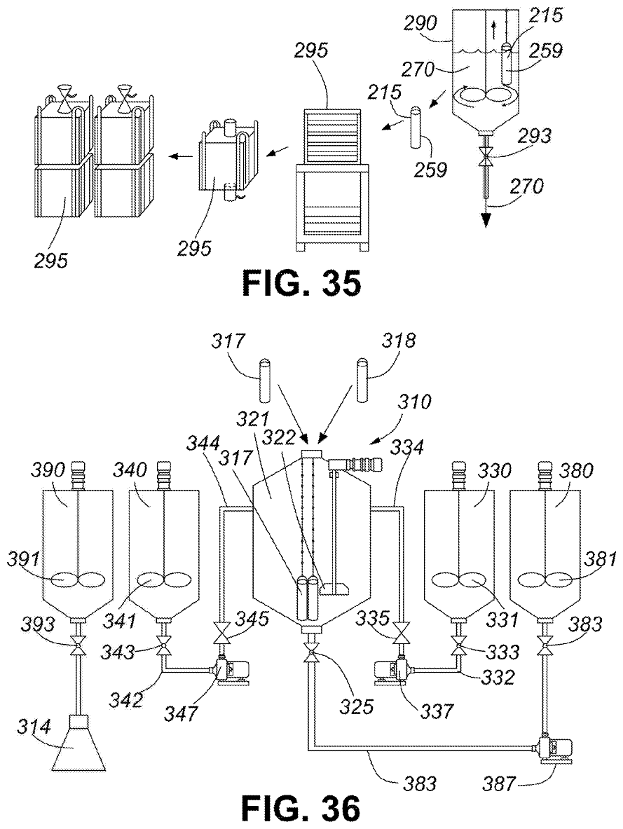

The wash vessel 290 need not be in fluid communication with the binding vessel 221. Fluid communication between the wash vessel 290 and the recovery vessel 214 may be provided and broken by a wash vessel valve 293. The immersion filter 215 may be immersed in the lipophilic solvent 260 in the wash vessel 290 for recovery of the recovered lipophilic target compound 259 in the recovery vessel 214, as shown in FIG. 34. The immersion filter 215 may be immersed in the binding solvent 258 or the hydrophilic solvent 270 in the wash vessel 290 for washing the immersion filter 215 to maintain binding between the lipophilic target substance and the insoluble polysaccharide bound with or otherwise adhered to, or sequestered within, the immersion filter 215, as shown in FIG. 35.

Immersion Filter Capture Protocol

FIGS. 30 to 35 show a system 210 in use to recover a lipophilic target compound using a housing, such as the immersion filter 215.

In FIG. 30, the immersion filter 215 containing the insoluble polysaccharide 250 is immersed into the lipophilic solvent 260 to wet the insoluble polysaccharide 250. The lipophilic solvent 260 may be provided to the binding vessel 221 from the lipophilic solvent vessel 230. The lipophilic solvent 260 may be provided in a ratio of 75% insoluble adsorbent 73 to 25% lipophilic solvent 260.

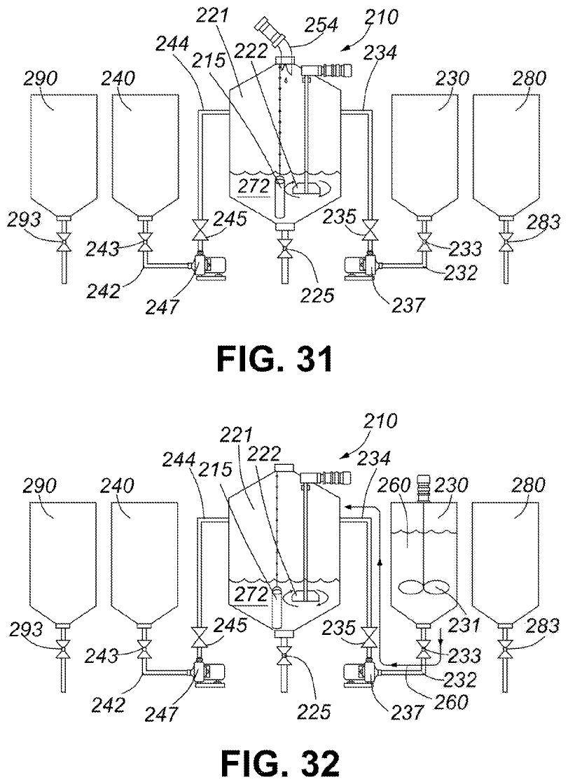

FIG. 31 shows the sample 254 being loaded into the binding vessel 221 and combined with the lipophilic solvent 260, providing the loaded solution 272. The binding vessel 221 may be chilled to between 3° C. and room temperature, such as to 4° C., when the sample 254 is added to the binding vessel 221. The sample 254 includes at least one lipophilic target compound.

FIG. 32 shows additional lipophilic solvent 260 being added to the binding vessel 221 to combine with the loaded solution 272. The additional lipophilic solvent 260 may dilute any water that may have been included in the sample 254. The additional lipophilic solvent 260 may facilitate dissolution into the loaded solution 272 of phytocannabinoids or other lipophilic target compounds in the sample 254. The loaded solution 272 may be agitated by the agitator 222.

FIG. 33 shows the hydrophilic solvent 270 being added to the binding vessel 221 and combined with the loaded solution 272 to provide the binding solvent 258 and to facilitate binding of the lipophilic target substance with the adsorbent in the immersion filter 215. The binding solvent 258 may target a ratio of lipophilic solvent 260 to hydrophilic solvent 270 selected to drive the lipophilic target compounds into the insoluble polysaccharide polymer core or otherwise bind with the insoluble polysaccharide contained in the immersion filter 215.

FIG. 34 shows the binding solvent 258 being drained into the flow-through vessel 280 via the flow-through line 282. FIG. 34 also shows removal of the immersion filter 215 from the binding vessel 221 and immersing the immersion filter 215 into the contents of the wash tank 290. In FIG. 34, the wash tank 290 contains the lipophilic solvent 260 for dissolving the lipophilic target compound in the lipophilic solvent 260. The lipophilic solvent 260 then drains through the wash vessel valve 293 and into the recovery vessel, where the recovered lipophilic target compound 259 may be removed and further processed into downstream products, for example as shown in FIG. 18.

The insoluble polysaccharide in the immersion filter 215 may be regenerated for reuse by emptying the insoluble polysaccharide from the immersion filter 215 and washing the insoluble polysaccharide in a detergent solution, for example 0.1% Triton X-100 at 37° C. for one minute. Solvents that are able to dissociate any lipophilic compounds from the insoluble polysaccharide, such as DMSO, may also be applied for regeneration. Exposure to the detergent solution, to solvent or other regeneration may be followed by re-equilibration with 3 to 5 volumes of ethanol. Alternatively, the insoluble polysaccharide may remain bound to or sequestered within the immersion filter 215 and regenerated in the immersion filter 215.

Lipophilic Target Compound Storage

FIG. 35 shows the insoluble polysaccharide 250 contained in the immersion filter 215 being stored in a storage system 295. The hydrophilic solvent 270 is provided to the wash vessel 290. The immersion filter 215 containing the insoluble polysaccharide bound with the recovered lipophilic target compound 259 is removed from the binding vessel 221 and placed in the wash vessel 290 for immersion in the hydrophilic solvent 270.

The hydrophilic solvent 270 in the wash vessel 290 with the immersion filter 215 immersed in the wash vessel 290 may be mixed for 1 hour to drive lipophilic target compounds into the insoluble polysaccharide polymer core or otherwise increasing adhering of the lipophilic target substance with the insoluble polysaccharide for washing the immersion filter 215 containing the insoluble polysaccharide 250. The hydrophilic solvent 270 may be drained from the wash vessel 290 for reuse or disposal.

The immersion filter 215 is removed from the wash vessel 290 and drained of hydrophilic solvent 270 by hanging to dry, exposing to airflow of atmospheric gases or of inert gases (e.g. argon, etc.) or low-reactivity gases (e.g. N₂, etc.).

After drying, the immersion filter 215 including the lipophilic target compound and the insoluble polysaccharide may be freeze dried or otherwise stabilized and stored in the storage system 295. The immersion filter 215 may be packaged for storage or transport once stabilized and stored, for example, in an opaque bag filled with inert gases (e.g. argon, etc.) or low-reactivity gases (e.g. N₂, etc.) for reducing oxidation nor UV light degradation. The immersion filter 215 may be removed from storage and eluted with the lipophilic solvent 260 or another lipophilic solvent to solubilize the recovered lipophilic target compounds 259 from the immersion filter 215 and recover the recovered lipophilic target compounds 259.

Multiple Immersion Filter Capture Setup

FIG. 36 shows a lipophilic compound recovery system 310. The system 310 includes the binding vessel 321. A first immersion filter 317 and a second immersion filter 318 that each include insoluble adsorbent are sized to be housed concurrently inside the binding vessel 321 for being in simultaneous or staged fluid communication with the binding vessel 321 and for receiving fluid from the binding vessel 321 to bind with the insoluble polysaccharide.

The first immersion filter 317 includes a first insoluble adsorbent and the second immersion filter 318 includes a second insoluble adsorbent. The first immersion filter 317 and the second immersion filter 318 are sized to receive filtrate that passes through the first immersion filter 317 and the second immersion filter 318, respectively. The binding vessel 321 is in fluid communication with the lipophilic solvent vessel 330 for receiving lipophilic solvent from the lipophilic solvent vessel 330. The binding vessel 321 is in fluid communication with the hydrophilic solvent vessel 340 for receiving hydrophilic solvent from the hydrophilic solvent vessel 340. The binding vessel 321 is in fluid communication with the flow-through vessel 380 for storing the binding solution (not shown; equivalent to the binding solution 258) after exposure of the sample (not shown; equivalent to the sample 254) to the first immersion filter 317 and the second immersion filter 318. The wash vessel 390 is in fluid communication with the recovery vessel 314 for receiving waste hydrophilic solvent (not shown; equivalent to the hydrophilic solvent 270) or binding solvent (not shown; equivalent to the binding solvent 258).

Each of the binding vessel 321, the lipophilic solvent vessel 330 and the hydrophilic solvent vessel 340 may be any suitable fluid vessel appropriate for the size, scale and application of the system 310 (e.g. a tank, pressure-rated tank, beaker, etc.).

The binding vessel 321 includes the agitator 322 positioned within the binding vessel 321. The agitator 322 is for agitating a fluid inside the binding vessel 321 to mix the fluid. The agitator 322 is shown as a rotary stirring agitator but any suitable agitator may be used (e.g. cross-flow, a venturi, static agitator, etc.). The binding vessel 321 is in fluid communication with the first immersion filters 317 and the second immersion filter 318 to provide direct contact with a solution in the binding vessel 321 (not shown; equivalent to the process shown for the system 210 in FIGS. 30 to 35).

The lipophilic solvent vessel 330 includes the agitator 331 positioned within the lipophilic solvent vessel 330. The agitator 331 is for agitating a lipophilic solvent inside the lipophilic solvent vessel 330 to mix the lipophilic solvent. The lipophilic solvent vessel 330 may be in fluid communication with the binding vessel 321 through the upstream lipophilic solvent flow line 332 and the downstream lipophilic solvent flow line 334. Fluid communication between the lipophilic solvent vessel 330 and the binding vessel 321 may be provided and broken by the upstream lipophilic solvent valve 333 and the downstream lipophilic solvent valve 335. Fluid communication between the lipophilic solvent vessel 330 and the binding vessel 321 may be driven by the pump 337.

The hydrophilic solvent vessel 340 includes the agitator 341 positioned within the hydrophilic solvent vessel 340. The agitator 341 is for agitating a hydrophilic solvent inside the hydrophilic solvent vessel 340 to mix the hydrophilic solvent. The hydrophilic solvent vessel 340 may be in fluid communication with the binding vessel 321, and correspondingly with the first immersion filter 317 and the second immersion filter 318, through the upstream hydrophilic solvent flow line 342 and the downstream hydrophilic solvent flow line 344. Fluid communication between the hydrophilic solvent vessel 340 and the binding vessel 321 may be provided and broken by the upstream hydrophilic solvent valve 343 and the downstream hydrophilic solvent valve 345. Fluid communication between the hydrophilic solvent vessel 340 and the binding vessel 321 may be driven by the pump 347.

The flow-through vessel 380 may be in fluid communication with the binding vessel 321 through the flow-through line 326. Fluid communication between the flow-through vessel 380 and the binding vessel 321 may be provided and broken by the output valve 325 and a flow-through valve 383. Fluid communication between the flow-through vessel 380 and the binding vessel 321 may be driven by the pump 387.

The flow-through vessel 380 may be in fluid communication with the first immersion filter 317 and the second immersion filter 318 through the upstream lipophilic solvent flow line 326 when the first immersion filter 317 and the second immersion filter 318 are immersed in the liquid contents of the binding vessel 321, for example, the binding solvent (not shown; equivalent to the binding solvent 258). Fluid communication between the flow-through vessel 326, the first immersion filter 317 and the second immersion filter 318, may be provided and broken by the flow-through valve 383 and by contact between the first immersion filter 317 and the second immersion filter 318, and the contents of the binding vessel 321. Fluid communication between the flow-through vessel 380, and the first immersion filter 317 and the second immersion filter 318, may be driven by the pump 387.

The wash vessel 390 need not be in fluid communication with the binding vessel 321. Fluid communication between the wash vessel 390 and the recovery vessel 314 may be provided and broken by a wash vessel valve 393. The first immersion filter 317 and the second immersion filter 318 may be immersed in the lipophilic solvent (not shown; equivalent to the lipophilic solvent 260) in the wash vessel 390 for recovery of a recovered lipophilic target compound (not shown; equivalent to the lipophilic target compound 259) in the recovery vessel 314. The first immersion filter 317 and the second immersion filter 318 may be immersed in the binding solvent (not shown; equivalent to the binding solvent 258) or the hydrophilic solvent (not shown; equivalent to the hydrophilic solvent 270) in the wash vessel 390 for washing the first immersion filter 317 and the second immersion filter 318 to maintain binding between the lipophilic target substance and the insoluble polysaccharide bound with or otherwise adhered to, or sequestered within, the first immersion filter 317 and the second immersion filter 318.

Each of the first immersion filter 317 and the second immersion filter 318 may include a distinct insoluble polysaccharide for binding to a respective distinct lipophilic target compound. Use of the first immersion filter 317 and the second immersion filter 318 or additional immersion filters simultaneously, may allow for the recovery of a plurality of lipophilic target compounds simultaneously. For example, unique lipophilic target compounds can be separately isolated from a plant extract sample as a result of preferential binding to the insoluble polysaccharide contained in each immersion filter.

Column Chromatography Capture Setup

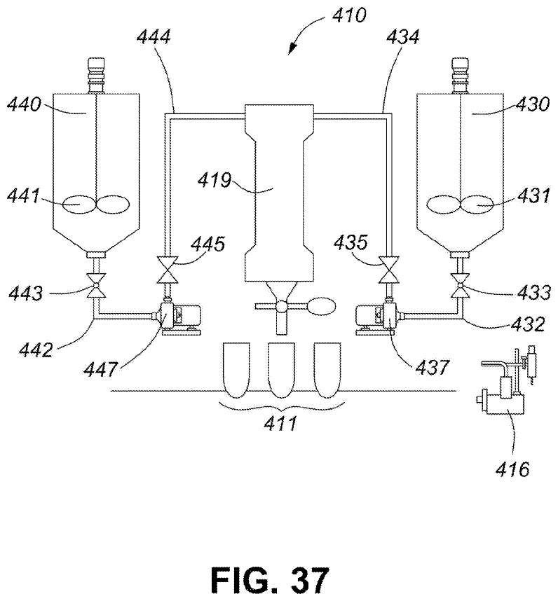

FIG. 37 shows a column chromatography system 410 for lipophilic compound recovery. A chromatography column 419 is in fluid communication with the lipophilic solvent vessel 430 and the hydrophilic solvent vessel 440. Each of the lipophilic solvent vessel 430 and the hydrophilic solvent vessel 440 may be any suitable fluid vessel appropriate for the size, scale and application of the system 410 (e.g. a tank, pressure-rated tank, etc.). A fractional recovery system 411 or other suitable recovery system may be provided for receiving eluate from the chromatography column 419.

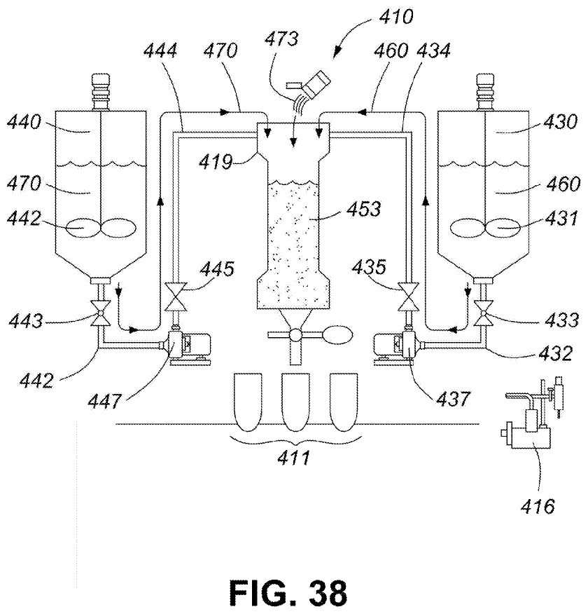

The lipophilic solvent vessel 430 includes the agitator 431 positioned within the lipophilic solvent vessel 430. The agitator 431 is for agitating a lipophilic solvent (e.g. the lipophilic solvent 460 as shown in FIG. 38) inside the lipophilic solvent vessel 430 to mix the lipophilic solvent. The lipophilic solvent vessel 430 is in fluid communication with the chromatography column 419.

The lipophilic solvent vessel 430 may be in fluid communication with the chromatography column 419 through the upstream lipophilic solvent flow line 432 and the downstream lipophilic solvent flow line 434. Fluid communication between the lipophilic solvent vessel 430 and the chromatography column 419 may be provided and broken by the upstream lipophilic solvent valve 433 and the downstream lipophilic solvent valve 435. Fluid communication between the lipophilic solvent vessel 430 and the chromatography column 419 may be driven by the pump 437.

The hydrophilic solvent vessel 440 may be in fluid communication with the chromatography column 419 through the upstream hydrophilic solvent flow line 442 and the downstream hydrophilic solvent flow line 444. Fluid communication between the hydrophilic solvent vessel 440 and the chromatography column 419 may be provided and broken by the upstream hydrophilic solvent valve 443 and the downstream hydrophilic solvent valve 445. Fluid communication between the hydrophilic solvent vessel 440 and the chromatography column 419 may be driven by the pump 447.

Column Chromatography Capture Protocol

FIGS. 38 to 44 show the system 410 in use to recover a recovered lipophilic target substance 449 using the insoluble adsorbent 473.

FIG. 38 shows the insoluble adsorbent 473 being provided to the chromatography column 419 and packed to provide a stationary phase 453. The insoluble polysaccharide includes a carbohydrate, silica or other matrix as further described at FIGS. 45 to 48.

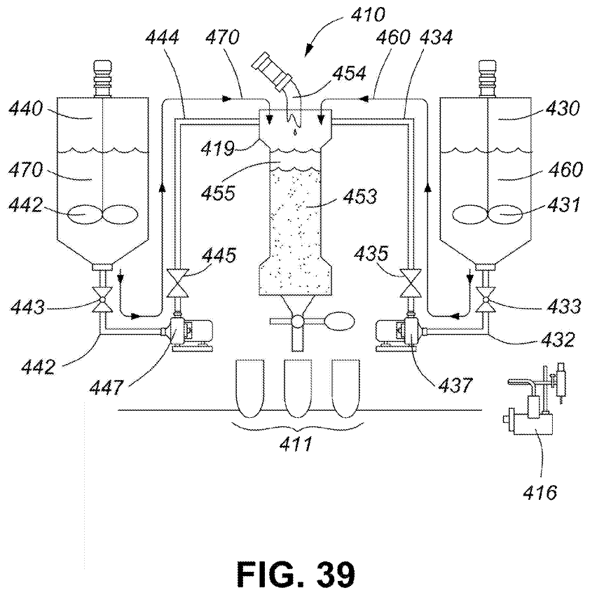

FIG. 39 shows the sample 454 being loaded into the chromatography column 419 for interacting with the stationary phase 453. The sample 454 may comprise a lipophilic target compound and may be dissolved in the binding solution (not shown; equivalent to the binding solution 258).

The sample 454 may flow into the chromatography column 419 and be eluted as a mobile phase 455. During flow and elution, the lipophilic solvent 460 and the hydrophilic solvent 470 may be provided to the chromatography column 419 in a proportion selected to facilitate binding of lipophilic target compounds in the sample 454 to the insoluble adsorbent 473 in the stationary phase 453.

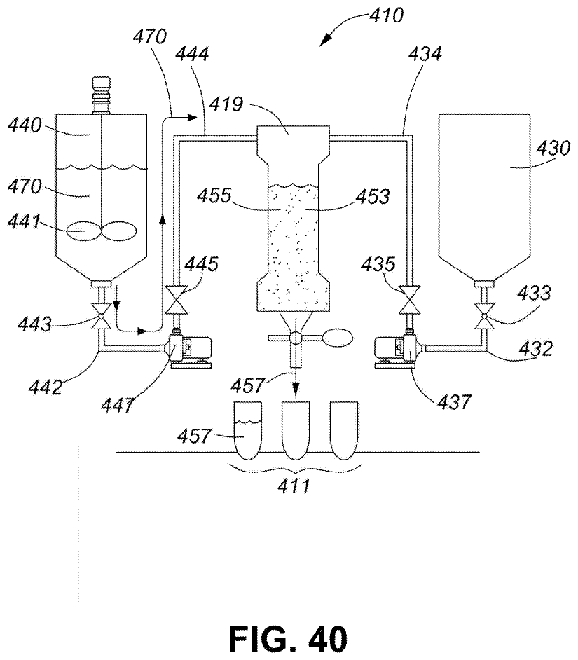

FIG. 40 shows the hydrophilic solvent 470 being added to the chromatography column 419 to further facilitate binding of lipophilic target compounds in the sample 454 to the insoluble adsorbent 473 in the stationary phase 453. The hydrophilic solvent 470 and the lipophilic solvent 460 may flow out of the chromatography column 419 and into the fractional recovery system 411 as eluate 457. The eluate 457 resulting from flow of the hydrophilic solvent 470 alone lacks any significant amount of the lipophilic target substance.

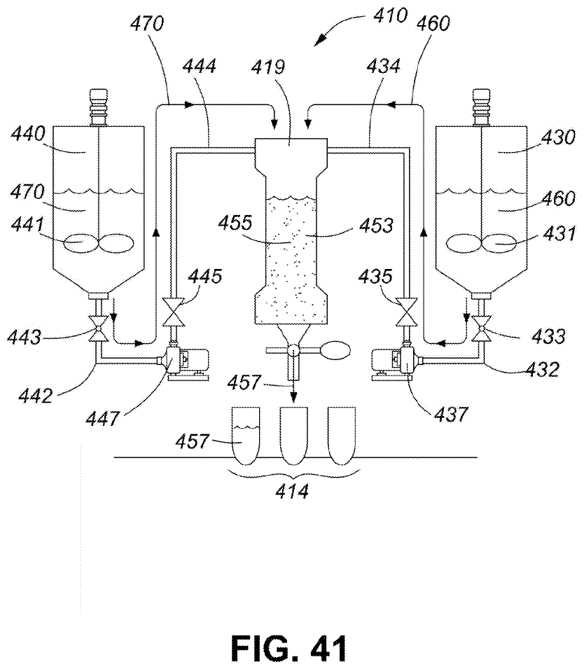

FIG. 41 shows the sample 454 being be eluted in the mobile phase 455 by a combination of the lipophilic solvent 460 or the hydrophilic solvent 470. The mobile phase 455 may contain an increasing proportion of the lipophilic solvent 460 over time as the elution progresses to provide the eluate 457 with an increasing amount of the lipophilic target substance. Fractions of the eluate 457 may be collected in separate vessels of the fractional recovery system 411 (e.g. test tubes, etc.). Lipophilic target compounds may be recovered from the fractions of the eluate 457 by known methods (e.g. example liquid-liquid extraction, evaporation, etc.)

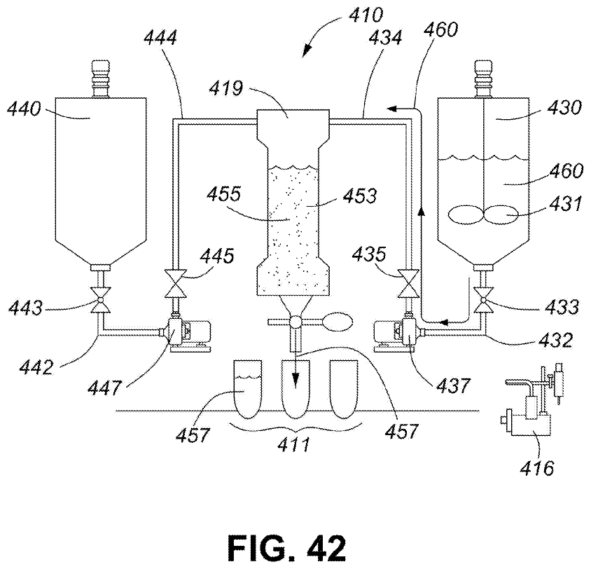

FIG. 42 shows the mobile phase 455 being eluted from the loaded chromatography column 419 with the lipophilic solvent 460 alone for dissociating the lipophilic target compounds from the cyclodextrin polymer stationary phase 453 and for solubilizing the lipophilic target compounds in the lipophilic solvent 460. The column filter 419 may be eluted in this manner until no more of the lipophilic target compound is eluted.

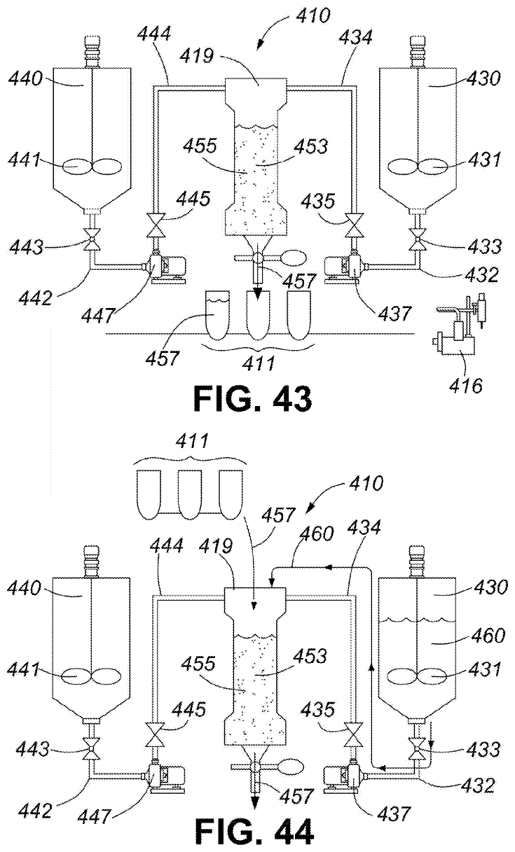

FIG. 43 shows the end of the elution cycle in which the mobile phase 455, at this point predominantly or entirely the lipophilic solvent 460, is drained into the fractional recovery system 411 as eluate 457.

FIG. 44 shows some fractions of the eluate 457 being provided back to the chromatography column 419 from the fractional recovery system 411 for further purification.

The insoluble adsorbent 473 may be regenerated for reuse by washing the insoluble adsorbent 473 with a detergent solution, for example 0.1% Triton X-100 at 37° C. for one minute. Solvents that are able to dissociate any lipophilic compounds from the insoluble adsorbent 473, such as DMSO, may also be applied for regeneration. Exposure to the detergent solution, to solvent or other regeneration may be followed by re-equilibration with 3 to 5 volumes of ethanol.

FIG. 45 shows an embodiment of the insoluble adsorbent 73 in which a polysaccharide-linker subunit 84 bound to an immobile matrix 88 (e.g. cellulose matrix, other carbohydrate matrix, silica matrix, etc.). The immobile matrix 88 may be used as the stationary phase 453 in the system 410. The polysaccharide-linker subunit 84 includes a cyclic polysaccharide 85 bound to a bidirectional linker 86. This embodiment includes at least two polysaccharide subunits 84 (i.e. n=2 or more).

FIG. 46 shows an embodiment of the insoluble adsorbent 73 in which the polysaccharide-linker subunit 84 includes 2,4-tolyl-diisocyanate as the bidirectional linker 86 and cellulose as the immobile matrix 88. This embodiment includes at least two polysaccharide-linker subunits 84 (i.e. n=2 or more).

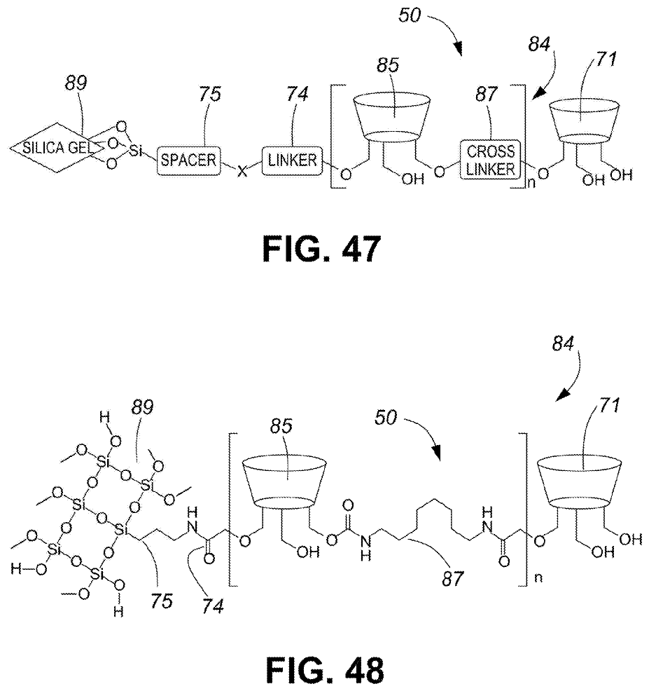

FIG. 47 shows an embodiment of the insoluble adsorbent 73 in which the polysaccharide-linker subunit 84 includes a cross-linker 87 and the cyclic polysaccharide 85. The polysaccharide-linker subunit 84 is bound to the linker 74 and the spacer 75. A silica-based immobile matrix 89 (e.g. a silica gel, mesomorphous silica, amorphous silica, etc.). This embodiment includes at least one polysaccharide-linker subunit 84 (i.e. n=1 or more), in addition to the terminal polysaccharide 71.

FIG. 48 shows an embodiment of the insoluble adsorbent 73 in which the polysaccharide-linker subunit 84 includes hexamethylene dicarbamate as the cross-linker 87 (which may be reacted from an isocyanate moiety), an amide as the linker 74 and a propyl group as the spacer 75. The immobile matrix 89 is a silica gel (e.g. mesomorphous silica, amorphous silica, etc.). This embodiment includes at least one polysaccharide-linker subunit 84 (i.e. n=1 or more), in addition to the terminal polysaccharide 71.

Any of the embodiments of the insoluble adsorbent 73 shown in FIGS. 45 to 48, or other immobilized insoluble adsorbent 73, may be used as the insoluble adsorbent 473 in the system 410.

Standard Protocol

A standard protocol was followed in all Examples with the variances from the standard protocol as described in each Example. The standard protocol included a plurality of steps. A mass of CBD was dissolved in ethanol to form a stock solution. A reference sample of the stock solution was diluted with ethanol to obtain a target CBD or other target molecule concentration for a reference measurement. A reaction sample was taken from the remaining stock solution. A cross-linked polysaccharide was combined with the reaction sample in a ratio relative to the CBD or other target molecule concentration present in the reaction sample (by mass) as specified in Examples. The cross-linked polymer is HDI-linked cyclodextrin prepared with a ratio of 8:1 HDI to cyclodextrin.

Water is combined with the reaction sample until the reaction sample reaches a target CBD or other target molecule concentration, and a target ethanol to water ratio. The reaction mixture including the water is filtered at a cutoff size of between 75 μm to 4,000 μm aperture size, or exposed to a magnetic field with a neodymium magnet through the wall of a flask, to retrieve the cross-linked polymer bound with CBD or other target molecule concentration. Once retrieved, the cross-linked polymer is flushed with a dissociation fluid for dissolving the target molecule. The dissociation fluid applied in the examples may be methanol, ethanol, isopropanol, a mixtures of aliphatic, aromatic and CO₂ fluids, DMSO, butane.

### Example 1

Ten milligrams of CBD were dissolved in a 10 mL mixture of 1:1 ethanol to water to produce a reaction mixture with a CBD concentration of 1 mg/mL. A 1 mL aliquot was then taken from the reaction mixture as a reference sample (t=0). One hundred milligrams of the cross-linked polymer was then combined with the reaction mixture for a polymer to CBD ratio of about 10:1 by mass. The reaction mixture was then stirred at room temperature.

One milliliter aliquots were then taken from the reaction mixture and filtered using pipette filtration at 10 minute intervals over 80 minutes (t=10, t=20, t=30, t=40, t=50, t=60, t=70, t=80, t=90). CBD capture data was obtained from the supernatant fluid of these aliquots after filtration. The data point at t=80 was obtained using syringe filtration, which may have filtered out more of the detectable CBD independently of the insoluble polysaccharide through adsorption. The t=90 time point returns to a level consistent with the time points beginning with t=30. About 15% of the CBD, or 1.7 mg was captured with a 10:1 polymer:CBD ratio.

After filtration and recovery of the cross-linked polymer, the cross-linked polymer was flushed with DMSO.

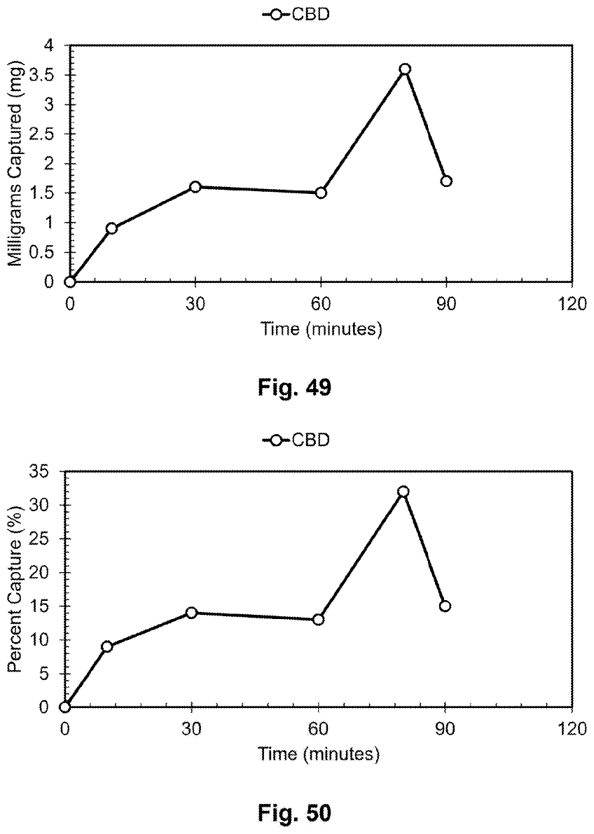

FIGS. 49 and 50 show the milligrams of the CBD captured and percent of total CBD captured, respectively, over the 80 minutes.

### Example 2

The protocol of Example 1 was followed. Sixty-eight milligrams of CBD were dissolved in a 1:1 mixture of ethanol and water to produce the reaction mixture with a CBD concentration of 1 mg/mL. Six-hundred and eighty-three milligrams of the cross-linked polymer were then combined with the reaction mixture for a polymer to CBD ratio of approximately 10:1 by mass. The capture was 11.6 mg of the 68 mg of CBD, or about 17%.

Filtration was performed by vacuum filtration using a Büchner funnel.

The cross-linked polymer was collected following filtration and divided into three portions. The first portion was combined with isopropyl alcohol (“IPA”) at room temperature, the second with IPA with the application of sonication/heat, and the third with DMSO at room temperature.

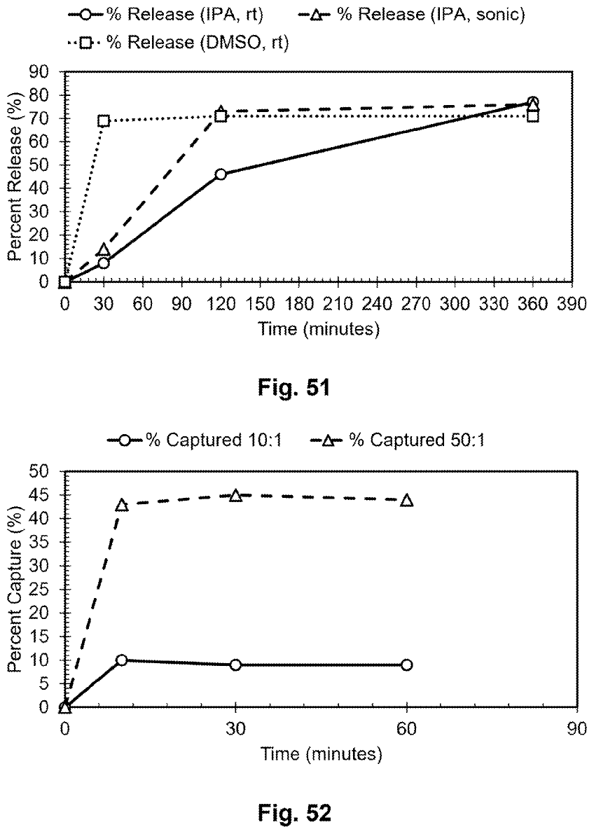

FIG. 51 shows the percentage of CBD release over time for each solvent.

### Example 3

The protocol from Example 1 was followed for a first batch with a polymer to CBD ratio of 10:1 by mass. For a second batch, the protocol from Example 1 was followed with a greater amount of cross-linked polymer to reach a polymer to CBD ratio of about 50:1 by mass. Pipette filtration was performed using aliquots collected over the course of more than 100 minutes. After 10 minutes, the 50:1 ratio showed about 4 to 5 times as much capture—about 40 to 45%, or 4.0 to 4.5 mg of CBD for 500 mg of polymer.

FIG. 52 shows the percentage of CBD captured over time.

### Example 4

The protocol of Example 1 was followed with the additional combination of 10 mg of cannabigerol (“CBG”) in the reaction mixture. The cross-linked polymer was combined in a ration of polymer to (CBD and CBG) of 25:1, with 500 mg of polymer to 20 mg of combined CBG and CBD. The dissociation fluid was DMSO. CBG capture was about 45 to 50% (4.5 to 5.0 mg of CBG for 500 mg of polymer). CBG was released into DMSO at room temperature with 104% recovery. No significant selectivity was observed between CBD and CBG. The polymer captured about double the phytocannabinoid weight compared with Example 3.

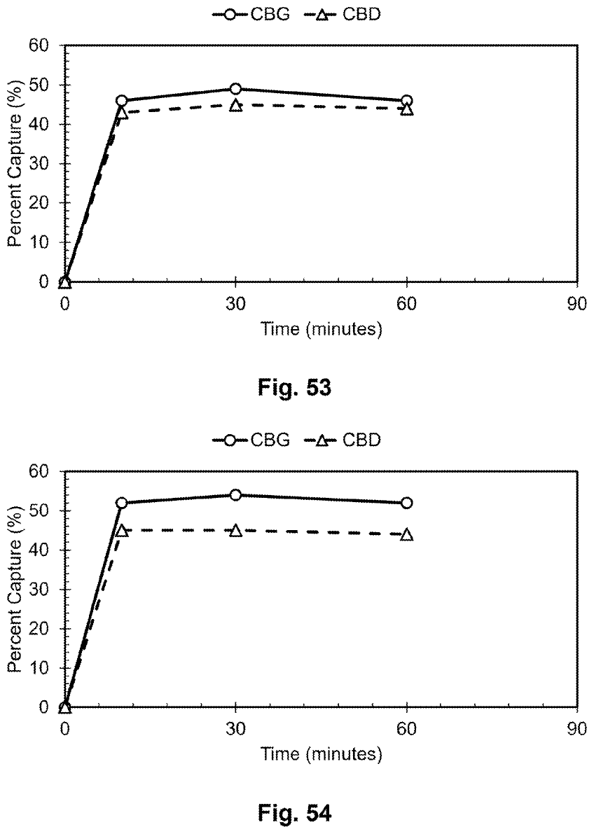

FIG. 53 shows the percentage of CBD and CBG captured over time in two separate experiments Example 4.

FIG. 54 shows the percentage of CBD and CBG captured over time when CBD and CBG are in competition in Example 4.

### Example 5

The protocol of Example 1 was followed for four batches. The first batch had an additional combination of 10 mg of vanillin in the reaction mixture. The second batch had an additional combination of 10 mg of olivetol in the reaction mixture. The third batch included 10 mg of vanillin and no CBD. The fourth batch combined 10 mg of olivetol and no CBD. CBD, vanillin and olivetol were recovered.

For each batch, 500 mg of the polymer was combined with the reaction mixture for a polymer to target molecule ratio of 50:1 by mass. Where there is more than one target molecule, the cross-linked polymer was combined with the reaction mixture in a polymer to CBD ratio of 50:1 by mass.

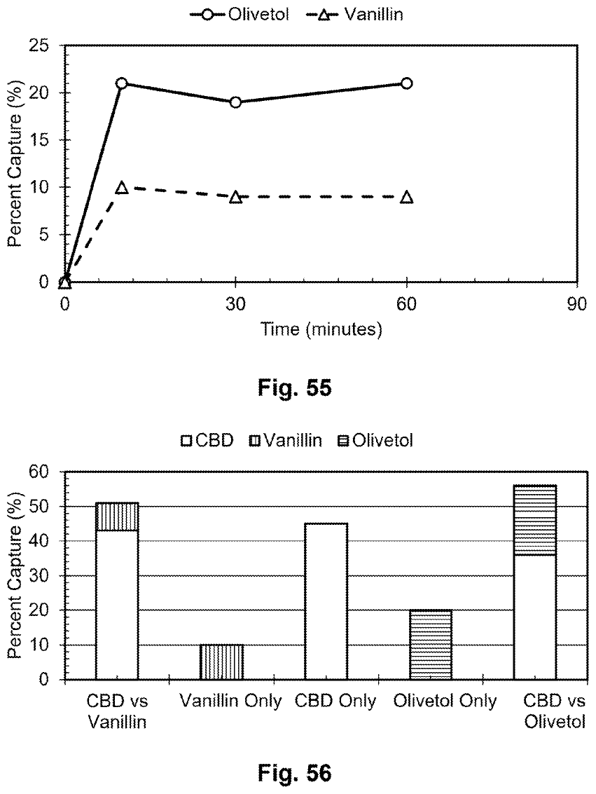

FIG. 55 shows the percentage capture of olivetol and vanillin over a period of 60 minutes.

FIG. 56 shows the percentage capture of CBD, vanillin and olivetol as at to 30 minutes.

### Example 6

Five batches of reaction mixture were prepared according to the protocol from Example 1 with the changes described below.

The first batch was prepared by combining 10 mg of CBD with 10 mL of 1:1 ethanol and water to reach a concentration of 1 mg/mL and a polymer to CBD ratio of 10:1 by mass.

The second batch was prepared by combining 10 mg of CBD with 5 mL of 1:1 ethanol and water to reach a concentration of 2 mg/mL and a polymer to CBD ratio of 10:1 by mass.

The third batch was prepared by combining 10 mg of CBD with 10 mL of 1:1 ethanol and water to reach a concentration of 1 mg/mL and a polymer to CBD ratio of 5:1 by mass.

The fourth batch was prepared by combining 20 mg of CBD with 10 mL of 1:1 ethanol and water to reach a concentration of 2 mg/mL and a polymer to CBD ratio of 10:1 by mass.

The fifth batch was prepared by combining 20 mg of CBD with 10 mL of 1:1 ethanol and water to reach a concentration of 2 mg/mL and a polymer to CBD ratio of 5:1 by mass.

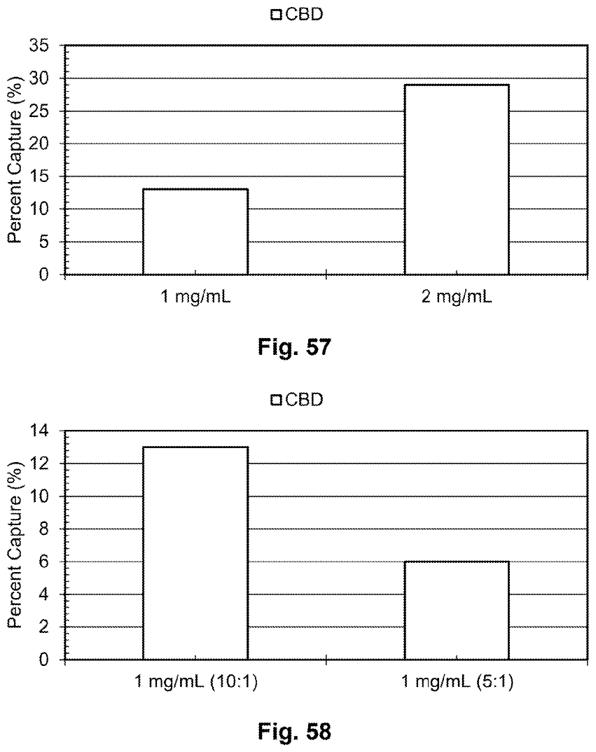

FIG. 57 shows the percentage of CBD captured in batches 1 and 2.

FIG. 58 shows the percentage of CBD captured in batches 1 and 3.

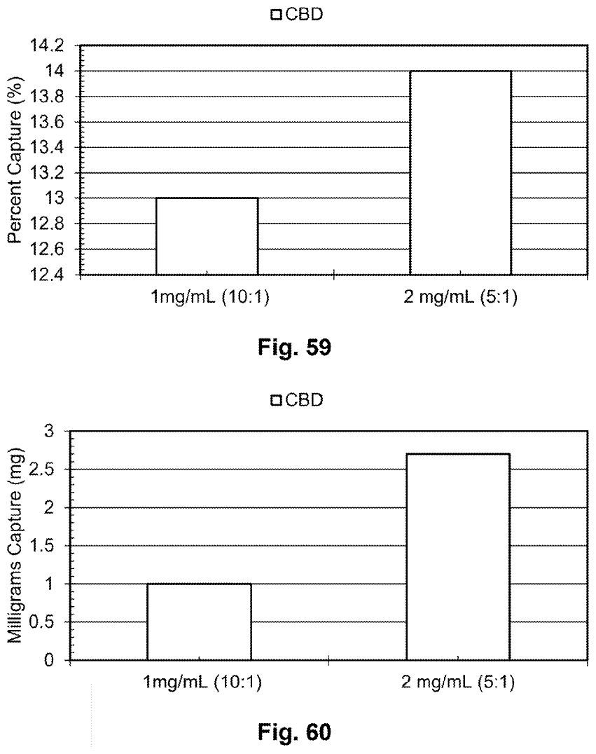

FIG. 59 shows the percentage of CBD captured in batches 1 and 4.

FIG. 60 shows the milligrams of CBD captured in batches 1 and 5 in milligrams.

### Example 7

The protocol from Example 1 was followed for a first batch having an initial CBD concentration of 2 mg/mL (10 mg CBD in 5 mL 1:1 ethanol and water) and a 10:1 polymer to CBD ratio by mass. A second batch was prepared using the protocol from Example 1 having a concentration of 2 mg/mL (10 mg CBD in 5 mL 1:1 ethanol and water) and a 510:1 polymer to CBD ratio by mass. In Example 3 at 1 mg/mL, a 5× increased in polymer resulted in a 4 to 5 fold increased in percent CBD retention. In this case, at 2 mg/mL, only a 2.3 fold increase resulted, showing 68% recovery at 50:1 compared with 29% recovery at 10:1, suggesting that the saturation point of CBD in 1:1 ethanol:H₂O was being reached.

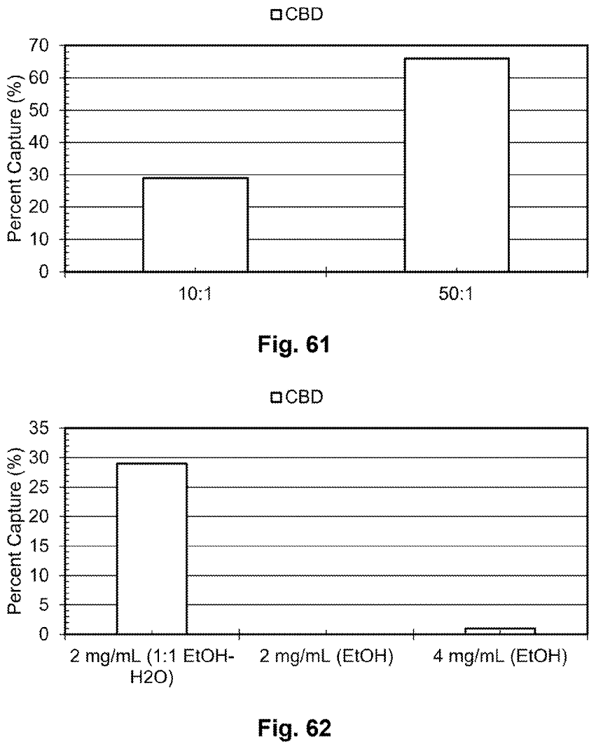

FIG. 61 shows the percentage of CBD captured for each batch.

### Example 8

The protocol from Example 1 was followed for a first batch having an initial CBD concentration of 2 mg/mL in 1:1 ethanol and water. A second batch was prepared of the same concentration in ethanol only. A third batch was prepared with a CBD concentration of 4 mg/mL in ethanol only. Each batch used a 10:1 polymer to CBD ratio by mass. In this case, there was no significant capture in EtOH of either 2 mg/mL or 4 mg/mL.

FIG. 62 shows the CBD percentage capture for each batch.

### Example 9

Sixty milligrams of CBD were dissolved in a 30 mL mixture of 1:1 ethanol to water to produce a stock solution with a CBD concentration of 2 mg/mL. The stock solution was divided into 6 portions. One portion of the stock solution was then taken to calculate a baseline CBD concentration at t=0.

A cross-linked polymer was then combined with each of the remaining five portions for a polymer to CBD ratio of about 10:1 by mass. It was then stirred at room temperature. A plateau was reached at about 5 minutes.

The remaining portions were each filtered at a different time interval two minutes apart (one at t=2, another at t=4, another at t=6, etc.). CBD capture data was obtained from the supernatant fluid of these portions after filtration.

After filtration and recovery of the cross-linked polymer, the cross-linked polymer was flushed with DMSO.

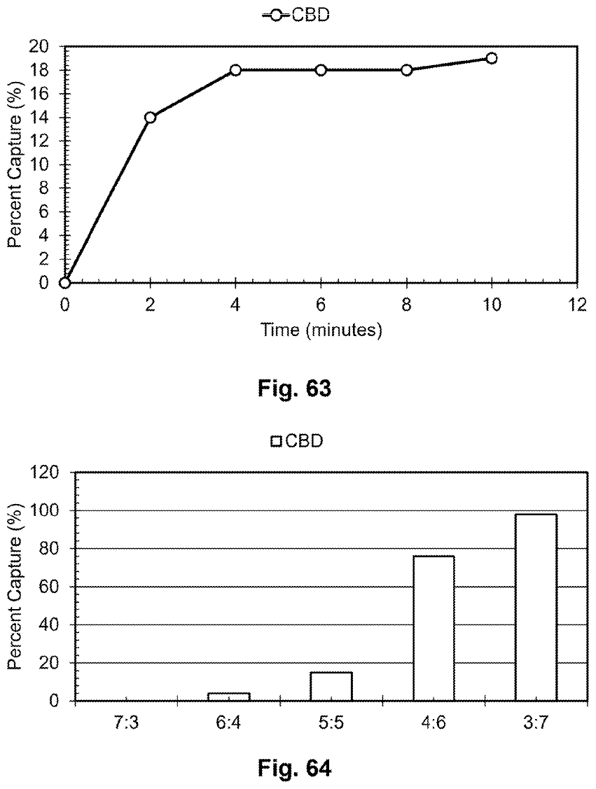

FIG. 63 shows the CBD capture over time where each data point is derived from a different portion.

### Example 10

The protocol of Example 9 was followed with the 60 mg initially dissolved in ethanol only. Water was then combined with each of the remaining five portions over the course of 2 to 5 minutes until the reaction mixture reached a target CBD concentration of 2 mg/mL. The remaining five portions were diluted to an ethanol to water ratio of 7:3, 6:4, 5:5, 4:6 or 3:7. The portions were then filtered to retrieve the cross-linked polymer.

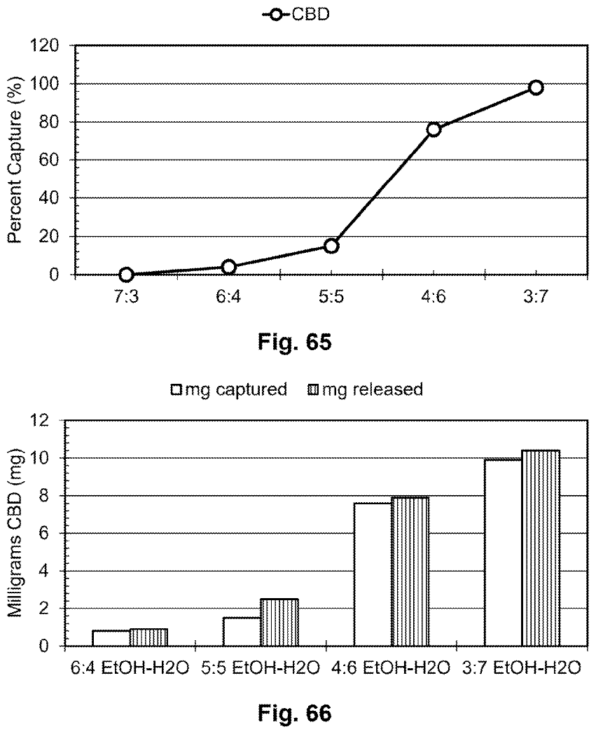

At 50:50 EtOH:H₂O, CBD dissolves provided that EtOH is added first then H₂O. At 45:56 EtOH:H₂O, the 2 mg/mL CBD solution is cloudy. At 40:60 EtOH:H₂O, the 2 mg/mL CBD is not fully dissolved. Slowly adding H₂O to the CBD, EtOH, polymer mixture results in very high capture of 98% at a 3:7 EtOH:H₂O. The S-shaped curve of FIG. 65 suggests insolubility-induced capture.

FIGS. 64 and 65 show the percentage of CBD capture over different ethanol to water ratios.

### Example 11

The protocol from Example 10 was followed, with filtration occurred a day after the diluted portions were prepared at EtOH:H₂O ratios of 6:4, 5:5, 4:6 and 3:7. The cross-linked polymer retrieved after filtration was then flushed with DMSO in a second reaction vessel.

FIG. 66 shows the milligrams of CBD captured and released for Example 11.

### Example 12

Ten milligrams of CBD were dissolved in 5 mL of a mixture of 1:1 ethanol to water to produce a stock solution with a CBD concentration of 2 mg/mL. One portion of the stock solution was then taken to calculate a baseline CBD concentration at t=0. The remaining stock solution was then divided into three portions.

Fresh cross-linked polymer was then combined with the first portion of stock solution. Recycled polymer was combined with the second portion or stock solution. Fresh cross-linked polymer was also combined with the third portion of stock solution. The polymer was combined such that each portion had a polymer to CBD ratio of about 10:1 by mass. It was then stirred at room temperature.

Each portion was filtered by pipette filtration. Once retrieved, the cross-linked polymer was flushed with ethanol. The percent of CBD captured of fresh cross-linked polymer was (23%) was comparable to that of reconstituted cross-linked polymer (24%). A second run of the fresh cross-linked polymer showed a consistent performance of 25% capture. This a similar performance to the conditions in Example 7, which showed 29% capture.

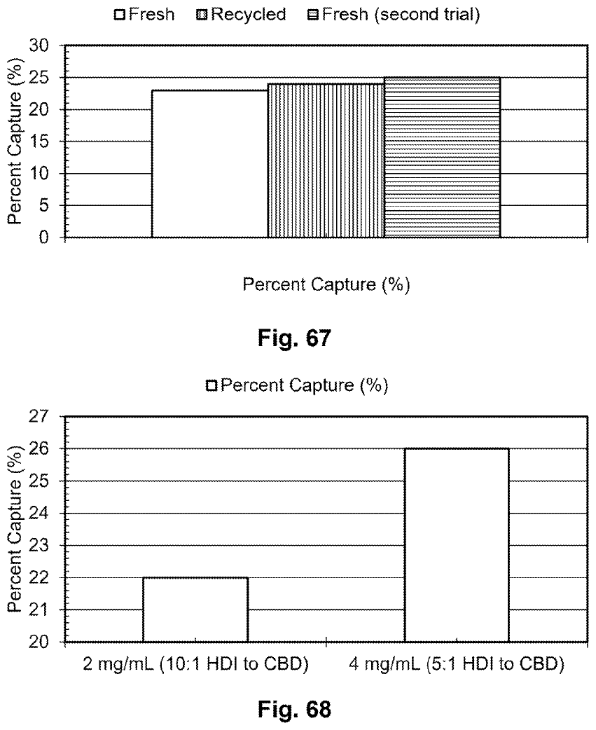

FIG. 67 shows the milligrams of CBD captured for Example 12.

### Example 13

Ten milligrams of CBD were dissolved in 5 mL of a mixture of 1:1 EtOH:H₂O to produce a first stock solution with a CBD concentration of 2 mg/mL.

A second reaction mixture was produced using a mass of CBD dissolved in a mixture of 1:1 EtOH:H₂O using sonication at room temperature to produce a stock solution with a CBD concentration of 4 mg/mL.

A third reaction mixture was produced using a mass of CBD was dissolved in a mixture of 1:1 EtOH:H₂O using sonication and heating to produce a stock solution with a CBD concentration of 6 mg/mL, but the solution did not dissolve.

One portion of each stock solution was then taken to calculate a baseline CBD concentration at t=0.

The first and second reaction mixtures were each divided into two batches. Cross-linked polymer was combined with the first batch of the first and second reaction mixtures in a ratio of 10:1 polymer to CBD by mass.

Cross-linked polymer was combined with the second batch of the first and second reaction mixtures in a ratio of 5:1 polymer to CBD by mass.

The batches were then filtered by pipette filtration and the recovered cross-linked polymer was flushed with ethanol as a dissociation fluid.

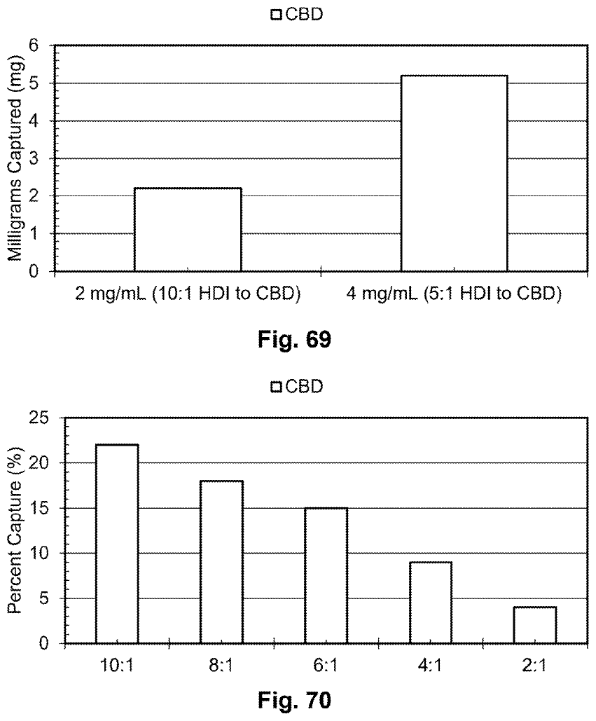

FIGS. 68 and 69 show the percentage and milligrams of CBD captured for Example 13.

### Example 14

A stock solution with a CBD concentration of 2 mg/mL was prepared according to the protocol set out in Example 12. The stock solution was divided into five vials each containing 10 mg of CBD. Cross-linked polymer was combined with each vial to produce polymer to CBD ratios of 10:1, 8:1, 6:1, 4:1 and 2:1, respectively. The vial contents were then filtered by pipette filtration and the recovered cross-linked polymer flushed with ethanol.

The percent captured is reduced as the ratio of cross linked polymer is reduced. The mg captured did not reach a plateau. The mg of cross linked polymer to mg of CBD captured was close to 45:1, varying to 40:1 at 6:1 polymer:CBD and to 50:1 at a polymer:CBD ratio of 2:1.

FIGS. 70 and 71 show the percentage and milligrams of CBD captured for Example 14, respectively. In FIG. 71, the ratio of cross-linked polymer to CBD captured is also shown on each data series.

### Example 15

A stock solution with a CBD concentration of 2 mg/mL was prepared according to the protocol set out in Example 12. The stock solution was divided into four vials, the first containing 5 mL of solution, the second containing 10 mL of solution, the third containing 15 mL of solution and the fourth containing 20 mL of solution. One hundred milligrams of cross-linked polymer were then combined to each vial.

One hundred milligrams of cross-linked polymer was combined with each vial to produce polymer to CBD ratios of 10:1, 5:1, 3.3:1, and 2.5:1, respectively.

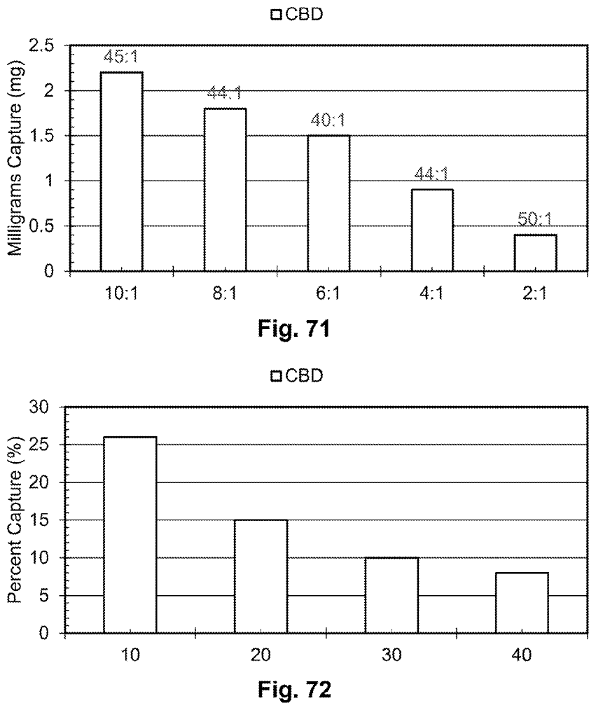

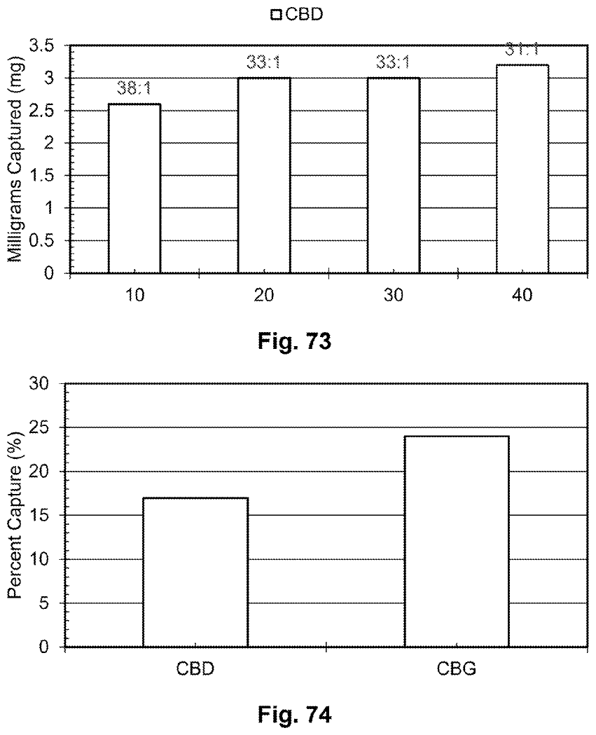

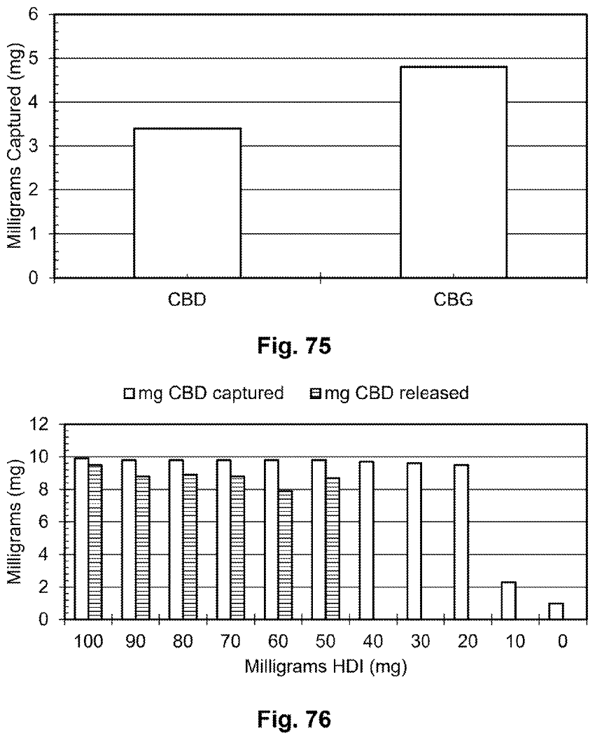

FIGS. 72 and 73 show the percentage and milligrams of CBD captured for Example 15, respectively. The percentage capture decrease as the amount of CBD increases, as expected. The mg captured reached a plateau at 3.2 mg. The mg of polymer to mg CBD was expected to plateau at 8:1.

### Example 16

Twenty milligrams of CBD and 20 mg of CBG were dissolved in a 1:1 mixture of ethanol and water to a concentration of 2 mg/mL. One hundred milligrams of cross-linked polymer were then combined to achieve a ratio of 5:1 polymer to CBD and 5:1 polymer to CBG by mass. The solution was then stirred at room temperature.

The vial contents were then filtered by pipette filtration and the recovered cross-linked polymer flushed with ethanol. In Example 4, 45% of the CBD was recovered and 54% of the CBG was recovered, a ratio of 1:1.2. In this Example, 17% of the CBD and 24% of the CBG was recovered, a ratio of 1:1.5. In total, 3.4 mg CBD and 4.8 mg CBG for 8.2 mg phytocannabinoids recovered.

FIGS. 74 and 74 show the percentage and milligrams of CBD and CBG captured in Experiment 16.

### Example 17

One hundred and twenty milligrams of CBD were dissolved in 18 mL in ethanol to form a stock solution.

The stock solution was divided into 1.5 mL portions. One portion of the stock solution was then taken to calculate a baseline CBD concentration at t=0. A cross-linked polymer was then combined with each of the remaining portions, each in a different quantity between 0 mg and 100 mg. The portions were stirred at room temperature. To all portions, 3.5 mL of water were then combined at a rate of 1 mL/minute.

The portions were filtered by pipette filtration to retrieve the cross-linked polymer. The cross-linked polymer was then flushed with DMSO. The saturation point was observed at a 2:1 polymer:CBD ratio. Ten percent of the CBD is not in solution when CBD in 3:7 EtOH:H₂O is used. The calculated cyclodextrin capacity within the polymer is 8:1 polymer:CBD. These results suggest that there are specific cyclodextrin encapsulated sites and also non-specific sites within the cyclodextrin polymer.

FIG. 76 shows the amount in milligrams of CBD captured and released in Example 17.

### Example 18

As stock solution was prepared according to Example 17. The stock solution was divided into 1.5 mL portions. One portion of the stock solution was then taken to calculate a baseline CBD concentration at t=0. Additional CBD was then combined with each portion in differing amounts of either 10 mg, 12.5 mg, 15.0 mg, or 17.5 mg. One hundred milligrams of cross-linked polymer was then combined with each of the remaining portions. To all portions, 3.5 mL of water was then combined with the portions at a rate of 1 mL/minute. Further water was combined with the portions such that a CBD concentration of 2 mg/mL and an ethanol to water ratio of 3:7 was achieved.

The portions were filtered by pipette filtration to retrieve the cross-linked polymer. The cross-linked polymer was then flushed with DMSO. There was over 98% capture in all cases. The calculated polymer capacity is 12.7 mg. These results suggest that there are specific cyclodextrin encapsulated sites and also non-specific sites within the cyclodextrin polymer.

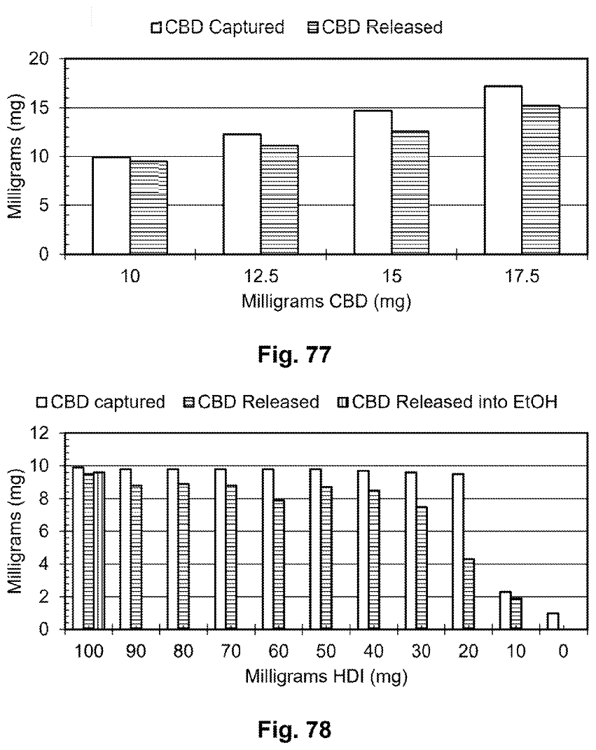

FIG. 77 shows the amount in milligrams of CBD captured and released in Example 18 as a function of the mg of CBD used.

### Example 19

A protocol was followed as in Example 17. The cross-linked polymer was flushed with DMSO.

FIG. 78 shows the amount in milligrams of CBD captured and released in Example 19.

### Example 20

Hops were extracted using the protocol set out in J. Inst. Brew., 1992, 98, 37-41. The extraction was performed using ethanol at a concentration of 300 g/L over 6 hours to produce a clear green solution. This ethanol extract of hops provides a simulated plant extract example.

One hundred milligrams of powdered hops were extracted using 5 mL of ethanol for thirty minutes, both at room temperature and with sonication to produce a clear green solution.

A protocol as in Example 17 was then followed using a polymer to CBD ratio of 10:1 by mass. The ethanol of Example 17 was replaced in two runs with the clear green solution of ethanol extract of hops that was obtained from the two hops extractions. The ethanol extract of hops showed 56% to 74% of the capture and about 52% of the release that was observed using pure ethanol, on a second trial about 50% of the release that was observed using pure ethanol. This result implies that there is some specificity of the cyclodextrin polymer for CBD over the compounds in hops at these concentrations.

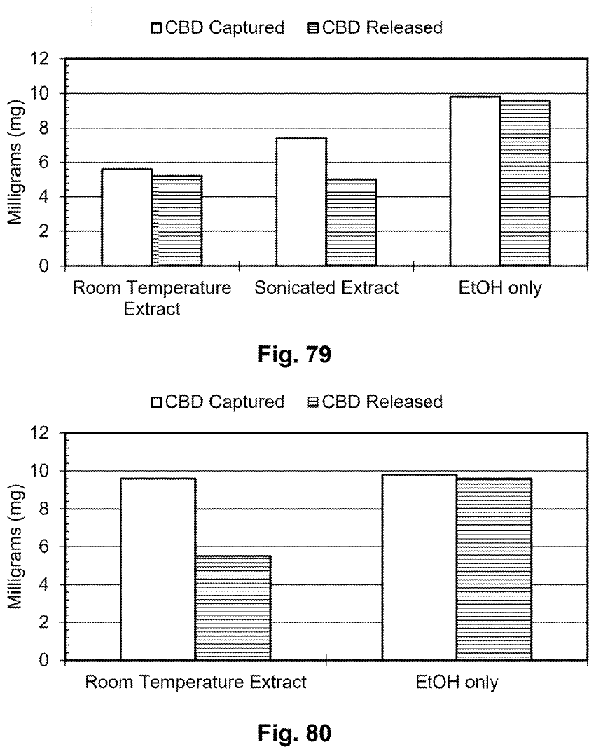

FIG. 79 shows the amount in milligrams of CBD captured and released in Example 20.

### Example 21

Three grams of powdered hops were extracted using 100 mL of ethanol for thirty minutes and then concentrated. The concentrate was then diluted with 10 mL of ethanol, filtered by pipette filtration to produce a clear green solution.

A protocol as in Example 20 was then followed using a polymer to CBD ratio of 10:1 and using the clear green solution obtained from the hops extractions in place of ethanol as well as with ethanol only. With pure ethanol, about 9.8 mg of CBD was captured and was released. With the ethanol extract of hops, 9.6 mg was captured and 5.5 mg was released. This result implies that there is some specificity of the cyclodextrin polymer for CBD over the compounds in hops at these concentrations.

Filtered was done by pipette filtration and the recovered cross-linked polymer flushed with ethanol.

FIG. 80 shows the amount in milligrams of CBD captured and released in Experiment 21.

### Example 22

Example 22 provide a protocol for removing colored impurities from ethanolic plant extracts prior to integration with the disclosed capturing method and protocol. It was visually observed that filtration through charcoal removed all detectable green pigments in the plant extract and the recovered lipophilic compound was less colored in appearance by comparison with material obtained according to Example 20 that did not include a charcoal decolorization process.

Fifty milligrams of CBD were dissolved in 7.5 mL of an ethanol extract of hops according to the protocol set out in Example 20. Seventy milligrams of charcoal was loaded into a pipette column formed using a cotton plug followed by 150 mg of Celite,® and the CBD solution was passed through the pipette in portions until all had been filtered. This stock solution was divided into 5 portions of 1.5 mL. A first portion of the stock solution was taken to calculate the baseline CBD concentration at t=0 by diluting with 3.5 mL of ethanol.

A second portion of the stock solution was transferred to a vial containing a stir bar and 100 mg of the cross-linked polymer derived from alpha-cyclodextrin. Water (3.5 mL) was then combined with the second portion over the course of 7 minutes at a rate of 0.5 mL/minute. The mixture was then stirred at room temperature for 30 minutes.

An aliquot was taken and filtered by pipette filtration to calculate the CBD concentration at t=30. The reaction mixture was then filtered to retrieve the cross-linked polymer. The cross-linked polymer retrieved after filtration was suspended in 5 mL ethanol. An aliquot was taken to determine released CBD concentration t=rel.

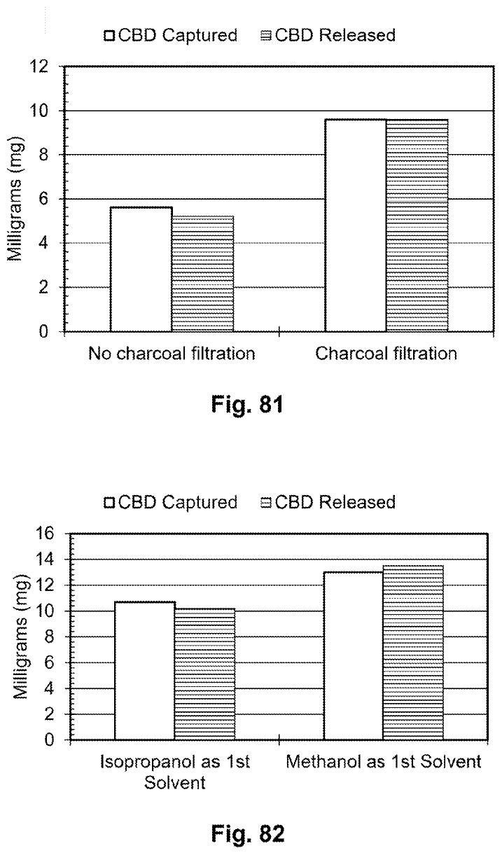

FIG. 81 shows the mass of captured and released CBD resulting from Example 22.

### Example 23

Thirty milligrams of CBD were dissolved in 4.5 mL of methanol to produce a CBD-methanol stock solution according to the protocol set out in Example 17. The CBD-methanol stock solution was divided into 3 portions of 1.5 mL. A first portion of the CBD-methanol stock solution was taken to calculate the baseline CBD concentration at t=0 by diluting with 3.5 mL of methanol.

Thirty milligrams of CBD were dissolved in 4.5 mL of isopropanol to produce a CBD-isopropanol stock solution according to the protocol set out in Example 17. The CBD-isopropanol stock solution was divided into 3 portions of 1.5 mL. A first portion of the CBD-isopropanol stock solution was taken to calculate the baseline CBD concentration at t=0 by diluting with 3.5 mL of isopropanol.

One portion of each stock solution was transferred to two separate vials containing a stir bar and 100 mg of the cross-linked polymer. To each portion, water (3.5 mL) was then combined over the course of 7 minutes at a rate of 0.5 mL/minute. The mixture was then stirred at room temperature for 30 minutes.

From each portion, an aliquot was taken and filtered by pipette filtration to calculate the CBD concentration at t=30. The reaction mixture was then filtered to retrieve the cross-linked polymer. The cross-linked polymers were retrieved after filtration was suspended in 5 mL ethanol. From both portions, aliquots were taken to determine released CBD concentration t=rel.

FIG. 82 shows the mass of captured and released of CBD resulting from Example 23.

These results imply that alcohols distinct from ethanol can be successfully used in conjunction with our capturing device to recover lipophilic compounds.

### Example 24

Thirty milligrams of CBD and thirty milligrams of CBG were dissolved in 4.5 mL of acetonitrile to produce a CBD-CBG-acetonitrile stock solution according to the protocol set out in Example 17. The CBD-CBG-acetonitrile stock solution was divided into 3 portions of 1.5 mL. One portion of the CBD-CBG-acetonitrile stock solution was taken to calculate the baseline CBD and CBG concentrations at t=0 by diluting with 3.5 mL of acetonitrile. One portion of the CBD-CBG-acetonitrile stock solution was transferred to a vial containing a stir bar and 100 mg of the cross-linked polymer.

Thirty milligrams of CBD and thirty milligrams of CBG were dissolved in 4.5 mL of acetone to produce a CBD-CBG-acetone stock solution according to the protocol set out in Example 17. The CBD-CBG-acetone stock solution was divided into 3 portions of 1.5 mL. One portion of the CBD-CBG-acetone stock solution was taken to calculate the baseline CBD and CBG concentrations at t=0 by diluting with 3.5 mL of acetone. One portion of the CBD-CBG-acetone stock solution was transferred to a vial containing a stir bar and 100 mg of the cross-linked polymer.

Thirty milligrams of CBD and thirty milligrams of CBG were dissolved in 4.5 mL of glycerol to produce a CBD-CBG-glycol stock solution according to the protocol set out in Example 17. The CBD-CBG-glycol stock solution was divided into 3 portions of 1.5 mL. One portion of the CBD-CBG-glycol stock solution was taken to calculate the baseline CBD and CBG concentrations at t=0 by diluting with 3.5 mL of glycerol. One portion of the CBD-CBG-glycol stock solution was transferred to a vial containing a stir bar and 100 mg of the cross-linked polymer.

To each of the above portions, water (3.5 mL) was then combined over the course of 7 minutes at a rate of 0.5 mL/minute. The mixtures were then stirred at room temperature for 30 minutes.

To each portion, an aliquot was taken and filtered by pipette filtration to calculate the CBD and CBG concentrations at t=30. The reaction mixtures were then filtered to retrieve the cross-linked polymer. The cross-linked polymer retrieved after filtration was suspended in 5 mL DMSO. An aliquot was taken to determine released CBD and CBG concentrations t=rel.

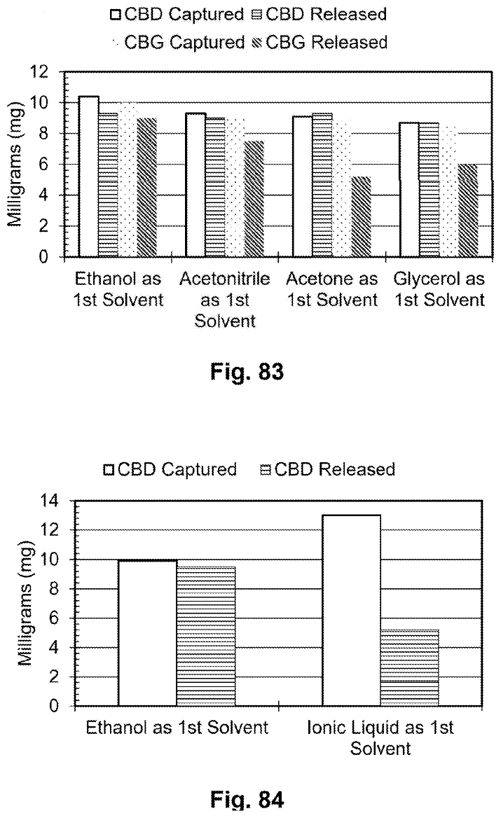

FIG. 83 shows the mass of captured and released of CBD and CBG resulting from Example 24. These results demonstrate that polar organic solvents other than ethanol, and specifically acetonitrile, acetone and glycerol, can be successfully used with the insoluble polysaccharides to recover the lipophilic compounds.

### Example 25

Thirty milligrams of CBD were dissolved in 4.5 mL of 1-butyl-3-methylimidazolium tetrafluoroborate, with considerable sonication due to viscosity, to produce a stock solution according to the protocol set out in Example 17. The stock solution was divided into 3 portions of 1.5 mL. A first portion of the stock solution was taken and diluted with 3.5 mL of ethanol to calculate the baseline CBD concentration at t=0.

A second portion of the stock solution was transferred to a vial containing a stir bar and 100 mg of the cross-linked polymer. Water (3.5 mL) was then combined with the second portion of the stock solution over the course of 7 minutes at a rate of 0.5 mL/minute. The reaction mixture was then stirred at room temperature for 30 minutes.

An aliquot of the reaction mixture was taken and filtered by pipette filtration to calculate the CBD concentration at t=30. The reaction mixture was then filtered to retrieve the cross-linked polymer. The cross-linked polymer retrieved after filtration was suspended in 5 mL ethanol. An aliquot was taken to determine released CBD concentration t=rel.

FIG. 84 shows the mass of captured and released of CBD resulting from Example 25.

These results demonstrate that solvents distinct from ethanol, specifically the ionic liquid 1-butyl-3-methylimidazolium tetrafluoroborate, can be successfully used as the first solvent for recovery of lipophilic compounds.

### Example 26

Sixty milligrams of CBD were dissolved in 9 mL of ethanol to produce a stock solution according to the protocol set out in Example 17. The stock solution was divided into 6 portions of 1.5 mL. A first portion of the stock solution was taken to calculate the baseline CBD concentration at t=0 by diluting with 3.5 mL of ethanol.

A second portion of the stock solution were transferred to a vial containing 100 mg of the cross-linked polymer derived from alpha-cyclodextrin. A third portion of the stock solution were transferred to a vial containing 100 mg of the cross-linked polymer derived from beta-cyclodextrin. A fourth portion of the stock solution were transferred to a vial containing 100 mg of the cross-linked polymer derived from gamma-cyclodextrin. To all portions, water (3.5 mL) was then combined with this portion over the course of 7 minutes at a rate of 0.5 mL/minute. The mixture was then stirred at room temperature for 30 minutes.

To all portions, an aliquot was taken from each vial and filtered by pipette filtration to calculate the CBD concentration at t=30. The reaction mixtures were then each filtered to retrieve the cross-linked polymers. The cross-linked polymers retrieved after filtration were suspended in 5 mL ethanol. An aliquot was taken to determine released CBD concentration t=rel.

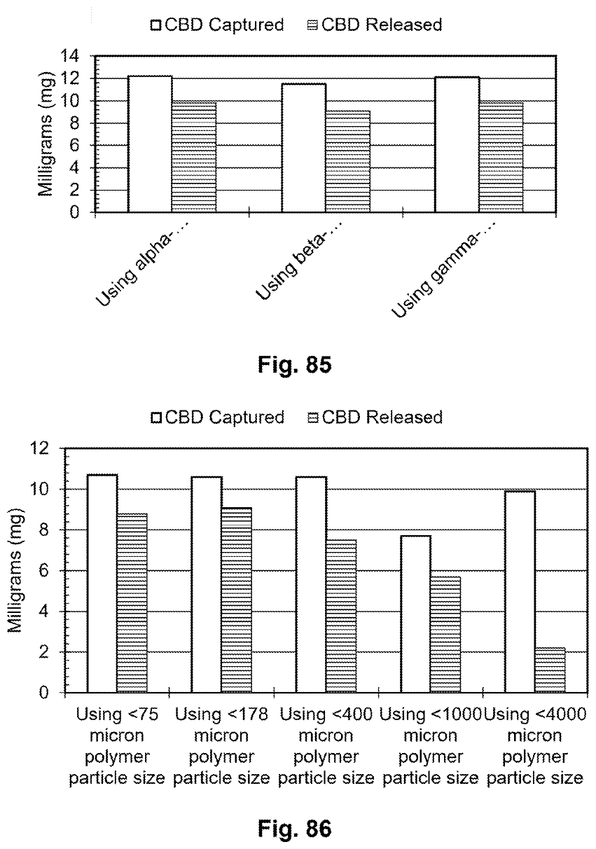

FIG. 85 shows the mass of captured and released of CBD resulting from Example 26. These results demonstrate that HDI-CDP derived from cyclic oligosaccharides distinct from beta-cyclodextrin, specifically alpha-cyclodextrin and gamma-cyclodextrin, can be applied to recover lipophilic compounds.

### Example 27

Seventy milligrams of CBD were dissolved in 10.5 mL of ethanol to produce a stock solution according to the protocol set out in Example 17. The stock solution was divided into 7 portions of 1.5 mL. A first portion of the stock solution was taken to calculate the baseline CBD concentration at t=0 by diluting with 3.5 mL of ethanol.

Five portions of the stock solution were each transferred into a respective vial, each containing a stir bar and cross-linked polymer ground to various particles sizes ranging from <75, <178, <400, <1000, <4000 microns. To all portions, water (3.5 mL) was then combined over the course of 7 minutes at a rate of 0.5 mL/minute. The mixture was then stirred at room temperature for 30 minutes.

From all portions, an aliquot was taken and filtered by pipette filtration to calculate the CBD concentration at t=30. The reaction mixture was then filtered to retrieve the cross-linked polymer. The cross-linked polymer retrieved after filtration was suspended in 5 mL DMSO. An aliquot was taken to determine released CBD concentration t=rel.

FIG. 86 shows the concentration of captured and released CBD resulting from Example 27. These results demonstrate that the cross-linked polymer can be deployed successfully using the protocol outlined in Example 27 when in the form of finely ground powder or macroscopic beads to recover lipophilic compounds. These results also demonstrate that smaller bead and particle sizes may have a greater lipophilic compound recovery potential than larger particle sized polymer.

### Example 28

Sixty milligrams of CBD were dissolved in 18.0 mL of ethanol to produce a stock solution according to the protocol set out in Example 17. The stock solution was divided into 6 portions of 3.0 mL. A first portion of the stock solution was taken to calculate the baseline CBD concentration at t=0 by diluting with 7.0 mL of ethanol.

Three portions of the stock solution were each transferred into three respective vials, the first containing 200 mg of the cross-linked polymer (400 micron mesh size) and an empty semi-permeable immersion filter, the second containing the same polymer housed within a semi-permeable immersion filter, the third containing the same polymer housed within a semi-permeable mesh netting connected to a string. To each portion, water (7 mL) was then combined with this portion over the course of 14 minutes at a rate of 0.5 mL/minute. The mixture was then stirred at room temperature for 30 minutes.

From each portion, an aliquot was taken and filtered by pipette filtration to calculate the CBD concentration at t=30. The cross-linked polymers were then retrieved by filtration. The immersion filter containing the cross-linked polymer was suspended in 10 mL ethanol. From each portion, an aliquot was taken to determine released CBD concentration t=rel.

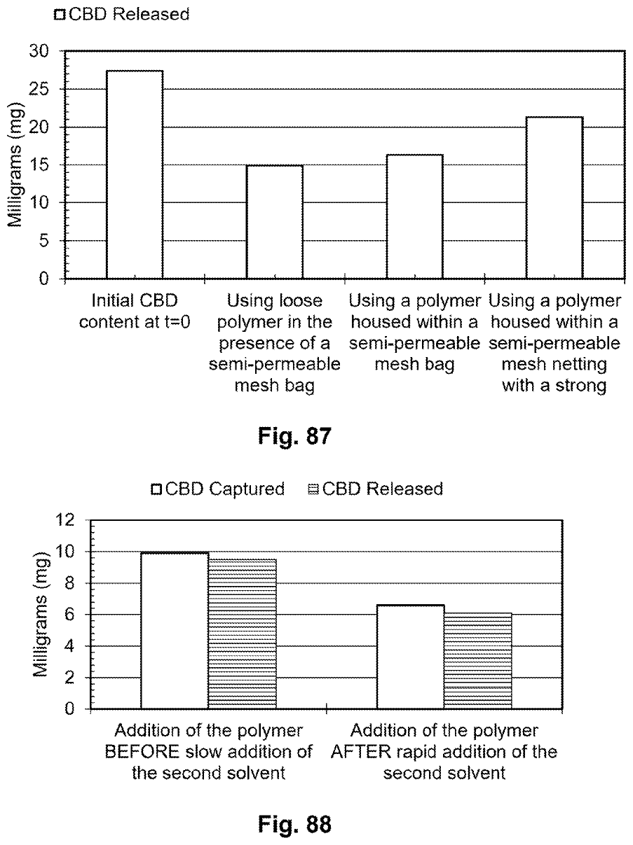

FIG. 87 shows the concentration of released CBD resulting from Example 28. These results demonstrate that one or more cross-linked polymers can be used while physically separated and housed within separate permeable mesh membranes.

### Example 29

Thirty milligrams of CBD were dissolved in 4.5 mL of ethanol to produce a stock solution according to the protocol set out in Example 17. The stock solution was divided into 3 portions of 1.5 mL. A first portion of the stock solution was taken to calculate the baseline CBD concentration at t=0 by diluting with 3.5 mL of ethanol.

A second portion of the stock solution was transferred to a vial containing a stir bar. The second portion was diluted using 3.5 mL of water to create a turbid suspension. 100 mg of the cross-linked polymer was combined with the reaction mixture. It was then stirred at room temperature for 30 minutes.

An aliquot was taken and filtered by pipette filtration to calculate the CBD concentration at t=30. The reaction mixture was then filtered to retrieve the cross-linked polymer. The cross-linked polymer retrieved after filtration was suspended in 5 mL DMSO. An aliquot was taken to determine released CBD concentration t=rel.

FIG. 88 shows the mass of captured and released CBD resulting from Example 29. These results demonstrate application to turbid suspensions of target compounds successfully. These data also demonstrate that slow addition of the second solvent to induce capture by the polymer may be more efficient than rapid addition.

### Example 30

Twenty milligrams of CBD were dissolved in 0.3 mL of ethanol and applied directly to a pipette column plugged with cotton and preloaded with 300 mg of a cross-linked polymer (<75 micron mesh size).

Water (5 mL) was applied to the column in five portions and collected in a single container following the application of compressed air to facilitate flow. An aliquot of this sample was taken to determine CBD concentration.

An ethanol-water mixture (2:8 EtOH:H₂O, 5 mL) was applied to the column in five portions and collected in a single container following the application of compressed air to facilitate flow. An aliquot of this sample was taken to determine CBD concentration.

An ethanol-water mixture (4:6 EtOH:H₂O, 5 mL) was applied to the column in five portions and collected in a single container following the application of compressed air to facilitate flow. An aliquot of this sample was taken to determine CBD concentration.

Ethanol (5 mL) was applied to the column in five portions and collected in a single container following the application of compressed air to facilitate flow. An aliquot of this sample was taken to determine CBD concentration.

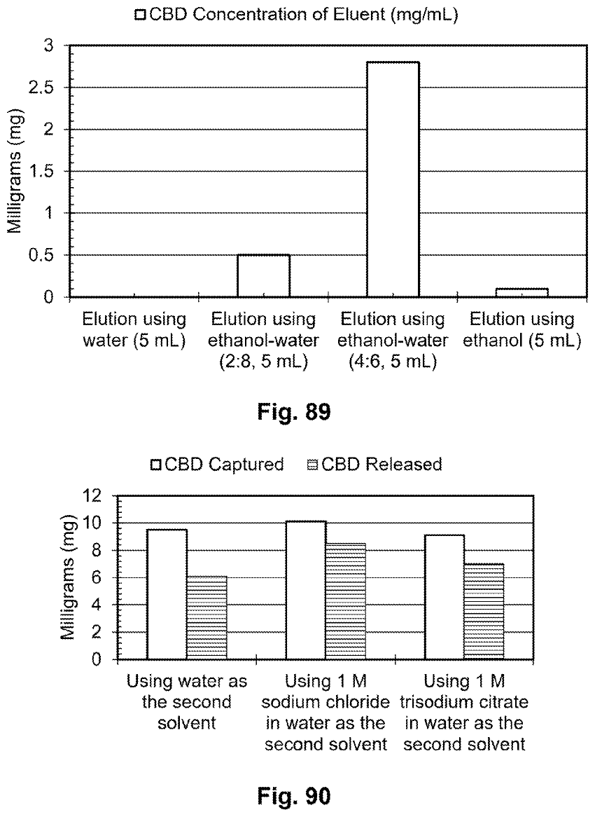

FIG. 89 shows the mass of released CBD resulting from Example 30. These results demonstrate use of the insoluble polysaccharide as a chromatography medium with gradient elution to retain and recover lipophilic compounds. These results also demonstrate that ethanol-water mixtures may be used to elute hydrophobic substances from the cross-linked polymeric chromatography medium.

### Example 31

Fifty milligrams of CBD were dissolved in 7.5 mL of an ethanol extract of hops according to the protocol set out in Example 20. The stock solution was divided into 5 portions of 1.5 mL. A first portion of the stock solution was taken to calculate the baseline CBD concentration at t=0 by diluting with 3.5 mL of ethanol.

Three portions of the stock solution were each transferred to three respective vials each containing a stir bar and 100 mg of the cross-linked polymer. To the first portion, water (3.5 mL) was added over the course of 7 minutes at a rate of 0.5 mL/minute. To the second portion, a sodium chloride solution in water (1 M, 3.5 mL) was added over the course of 7 minutes at a rate of 0.5 mL/minute. To the third solution, a trisodium citrate solution in water (1 M, 3.5 mL) was added over the course of 7 minutes at a rate of 0.5 mL/minute. The mixtures was then stirred at room temperature for 30 minutes.

From each portion, an aliquot was taken and filtered by pipette filtration to calculate the CBD concentration at t=30. The reaction mixture was then filtered to retrieve the cross-linked polymer. The cross-linked polymer retrieved after filtration each suspended in 5 mL ethanol. From each portion, an aliquot was taken to determine released CBD concentration t=rel.

FIG. 90 shows the mass of captured and released CBD resulting from Example 31. These results demonstrate that water-salt solutions may be used as the second solvent. These results also imply that a second solution with greater ionic strength may improve efficiency of lipophilic compound recovery. In addition, it was observed visually that lipophilic compound samples recovered using a brine solution as the second solvent contained fewer colored impurities.

### Example 32

Sixty milligrams of CBD were dissolved in 9.0 mL of an ethanolic hops extract according to the protocol set out in Example 20. The stock solution was divided into 6 portions of 1.5 mL. One portion of the stock solution was taken to calculate the baseline CBD concentration at t=0 by diluting with 3.5 mL of ethanol.

Four portions of the stock solution were transferred to four separate vials each containing a stir bar and 100 mg of a structurally distinct cross-linked polymer derived from reaction of beta-cyclodextrin and a different diisocyanate as cross-linking agent. The first portion was added to a vial containing polymer prepared using hexamethylene diisocyanate (HDI-CDP). The second portion was added to a vial containing polymer prepared using isophorone diisocyanate (IPI-CDP). The third portion was added to a vial containing polymer prepared using 4,4′-methylenebis(phenyl isocyanate) (MPI-CDP). The fourth portion was added to a vial containing polymer prepared using tolylene-2,4-diisocyanate (TDI-CDP).

To each portion, brine (1.0 M, 3.5 mL) was added over the course of 7 minutes at a rate of 0.5 mL/minute. The mixtures were then stirred at room temperature for 30 minutes. From each portion, an aliquot was taken and filtered by syringe filtration to calculate the CBD concentration at t=30. The reaction mixtures were then filtered to retrieve the cross-linked polymers. The cross-linked polymer retrieved after filtration each suspended in 5 mL ethanol. From each portion, an aliquot was taken to determine released CBD concentration t=rel.

FIG. 91 shows the mass of captured and released CBD resulting from Example 32. These results demonstrate that cross-linking agents other than hexamethylene diisocyanate can be employed to prepare insoluble polysaccharides for lipophilic compound recovery. This data also demonstrates that the efficacy of compound recovery may have structure-activity dependence and that the polymer prepared using hexamethylene diisocyanate was more effective than the three other cross-linking agents used in Example 32.

### Example 33

Fifty milligrams of CBD were dissolved in 7.5 mL of an ethanolic hops extract according to the protocol set out in Example 20. The stock solution was divided into 5 portions of 1.5 mL. One portion of the stock solution was taken to calculate the baseline CBD concentration at t=0 by diluting with 3.5 mL of ethanol.

Three portions of the stock solution were transferred to three separate vials each containing a stir bar and 100 mg of a cross-linked polymer derived from reaction of beta-cyclodextrin and hexamethylene diisocyanate at different CD:HDI molar ratios. The first portion was added to a vial containing polymer prepared using 1:8 CD to HDI. The second portion was added to a vial containing polymer prepared using 1:4 CD to HDI. The third portion was added to a vial containing polymer prepared using 1:2 CD to HDI.

To each portion, brine (1.0 M, 3.5 mL) was added over the course of 7 minutes at a rate of 0.5 mL/minute. The mixtures were then stirred at room temperature for 30 minutes. From each portion, an aliquot was taken and filtered by syringe filtration to calculate the CBD concentration at t=30. The reaction mixtures were then filtered to retrieve the cross-linked polymers. The cross-linked polymer retrieved after filtration each suspended in 5 mL ethanol. From each portion, an aliquot was taken to determine released CBD concentration t=rel.

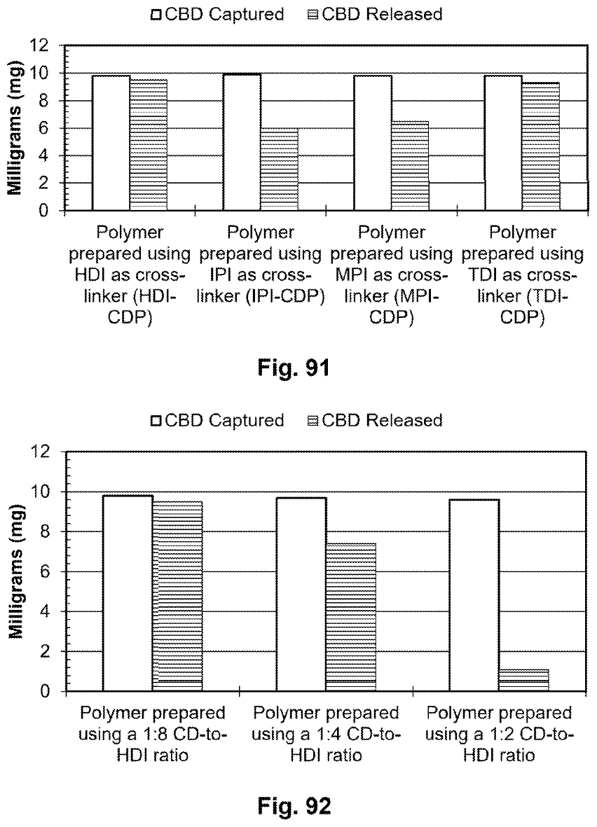

FIG. 92 shows the mass of captured and released CBD resulting from Example 33. These results demonstrate that the molar proportion of cross-linking agent used in preparation of the polymer relative to the cyclodextrin may influence lipophilic compound recovery. Specifically, the efficacy of lipophilic compound recovery was shown to be optimal at 1:8 CD-to-HDI ratio relative to 1:4 or 1:2 ratios of CD-to-HDI.

### Example 34

Dried plant material (4% moisture content) from the Carmagnola cultivar of cannabis hemp was determined to contain 2.69% total CBD (CBDA+CBD). Fresh plant matter (50.86 g, 72.5% moisture content) consisting of flower, buds, leaves, and small stems from the same source was finely chopped using shears and subsequently blended for 10 minutes in the presence of ethanol (250 mL) to produce a deep green solution and plant pulp.

The deep green solution and plant pulp was transferred to a round-bottomed flask fitted with a condenser and a stir bar. The mixture was heated to 70° C. and stirred at this temperature for a further 60 minutes before gradual cooling to room temperature. The mixture was filtered through filter paper using Buchner funnel, rinsing the residual plant material with additional ethanol to a final total volume of 340 mL to produce a stock solution.

From the stock solution, 6.0 mL was transferred to a vial containing 200 mg of the cross-linked polymer and a stir bar. Brine (1.0 M, 14.0 mL) was then combined with this portion over the course of 14 minutes at a rate of 1.0 mL/minute. The mixture was then stirred at room temperature for 30 minutes.

The reaction mixture was then filtered to retrieve the cross-linked polymer. The cross-linked polymer retrieved after filtration was suspended in 5 mL ethanol. An aliquot was taken to determine released total CBD concentration t=rel and compared with the theoretic maximum of recoverable total CBD based on dried plant matter analysis, adjusting for moisture content.

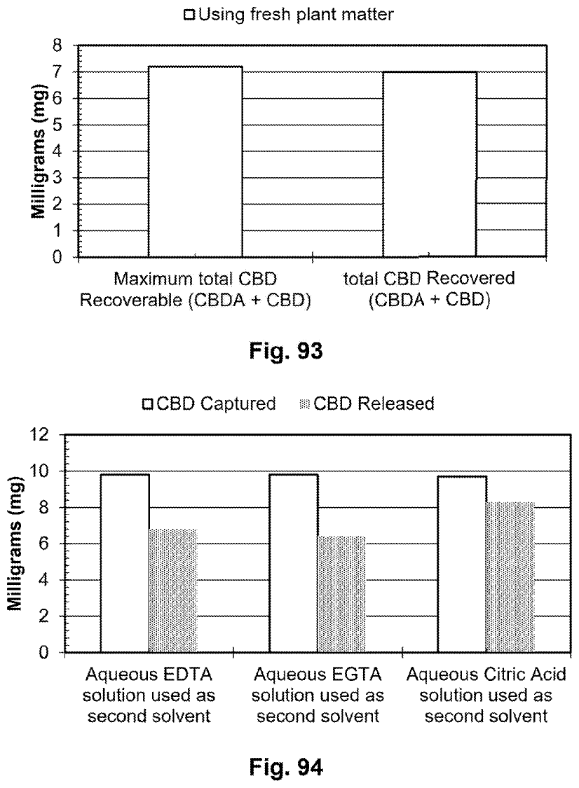

FIG. 93 shows the maximum total CBD recoverable and total CBD recovered using fresh plant matter. These results demonstrate that influence of moisture in fresh plant matter did not negatively affect the removal of CBD and CBDA from the biomass into ethanol when facilitated by vigorous blending, stirring, and application of heat.

### Example 35

Fifty milligrams of CBD were dissolved in 7.5 mL of an ethanolic hops extract according to the protocol set out in Example 20. The stock solution was divided into 5 portions of 1.5 mL. One portion of the stock solution was taken to calculate the baseline CBD concentration at t=0 by diluting with 3.5 mL of ethanol.

Three portions of the stock solution was transferred to three separate vials each containing a stir bar and 100 mg of the cross-linked polymer. To the first portion, an EDTA solution in water (1.0 M, 3.5 mL) was added over the course of 7 minutes at a rate of 0.5 mL/minute. To the second portion, EGTA solution in water (1 M, 3.5 mL) was added over the course of 7 minutes at a rate of 0.5 mL/minute. To the third solution, a citrate acid solution in water (1 M, 3.5 mL) was added over the course of 7 minutes at a rate of 0.5 mL/minute. The reaction mixtures were then stirred at room temperature for 30 minutes.

From each reaction mixture, an aliquot was taken and filtered by pipette filtration to calculate the CBD concentration at t=30. Each reaction mixture was then filtered to retrieve the cross-linked polymer. The cross-linked polymer retrieved after filtration each suspended in 5 mL ethanol. From each portion, an aliquot was taken to determine released CBD concentration t=rel.

FIG. 94 shows the mass of captured and released CBD resulting from Example 35. These results demonstrate that aqueous solutions of chelating agents may be used in place of water as the second solvent. These results also demonstrate that chelating agents influence the efficiency of lipophilic compound recovery in a structure-dependent manner and that citric acid was optimal within the range demonstrated above. In addition, it was observed visually that lipophilic compound samples recovered using an aqueous chelating agent solution as the second solvent contained fewer colored impurities when compared with using water alone.

### Example 36

A deep eutectic solvent mixture was formed using equimolar portions of acetic acid and (±)-menthol by heating to 70° C. for one hour. Sixty milligrams of CBD were dissolved in 9.0 mL of the deep eutectic solvent mixture and heating was maintained while dissolution occurred to form a stock solution. The stock solution was divided into 4 portions of 1.5 mL. One portion of the stock solution was taken to calculate the baseline CBD concentration at t=0 by diluting with 3.5 mL of ethanol.

One portion of the stock solution was transferred to vial each containing a stir bar and 100 mg of the cross-linked polymer. With the continuation of heating to 70° C. and stirring, water (3.5 mL) was added over the course of 7 minutes at a rate of 0.5 mL/minute. The reaction mixtures were then stirred at 30 minutes with continued heating.

An aliquot was taken and filtered using a syringe filter then used to calculate the CBD concentration at t=30. The reaction mixture was then filtered to retrieve the cross-linked polymer. The cross-linked polymer retrieved after filtration each suspended in 5 mL ethanol. An aliquot was taken to determine released CBD concentration t=rel.

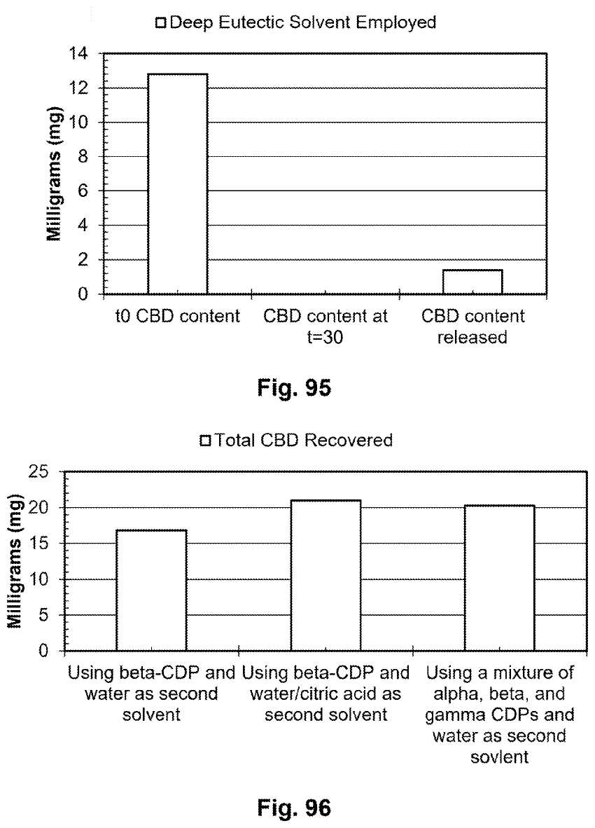

FIG. 95 shows the CBD content recovered resulting from Example 36. These results demonstrate that non-conventional solvents including a deep eutectic solvent derived from (±)-menthol-acetic acid may be used in place of ethanol as the lipophilic solvent.

### Example 37

One hundred and fifty milligrams of CBD were dissolved in 20 mL of an ethanol extract of hops according to the protocol set out in Example 20 to produce a stock solution. This stock solution was divided into 4 portions of 4.0 mL each. One portion of the stock solution was taken to calculate the baseline CBD concentration at t=0 by diluting with 9.0 mL of ethanol.

One portion of the stock solution was transferred to a vial containing a stir bar and three portions of 100 mg of cross-linked polymer derived from α-, β- and γ-cyclodextrin (<125 micron particle size). Water (9.0 mL) was then combined with this portion over the course of 9 minutes at a rate of 1.0 mL/minute. The mixture was then stirred at room temperature for 30 minutes.

A chromatography column was packed with 1.5 g of coarsely-ground (125-250 micron particle size) cyclodextrin polymers (comprising a mixture of equal proportions of polymers derived from α-, β- and γ-cyclodextrin) to a height of 3.5 cm with diameter 1 cm.

The chromatography medium was flushed with 20 mL ethanol followed by 20 mL water. The first reaction mixture was poured onto the chromatography medium, rinsing the vial with 30 mL water. The liquid was entirely forced through the media using a gentle application of compressed gas. A 10 mL portion of ethanol was added to the top of the column, and the liquid entirely forced through the media in the same manner and collected in a separate receptacle. Using additional portions of ethanol this process was repeated a total of eight times.

The CBD content of each fraction was determined and the eluents combined to a total of 100 mL ethanol. The CBD content of combined ethanol fractions was determined.

FIG. 96 shows total CBD recovered with different second solvents. These results demonstrate that polymers derived from α-, β- and γ-cyclodextrins may all be used for recovery of lipophilic compounds.

### Example 38

One hundred and fifty milligrams of CBD were dissolved in 20 mL of an ethanol extract of hops according to the protocol set out in Example 20 to produce a stock solution. The stock solution was divided into 4 portions of 4.0 mL each. One portion of the stock solution was taken to calculate the baseline CBD concentration at t=0 by diluting with 9.0 mL of ethanol.

One portion of the stock solution was transferred to a vial containing a stir bar and 300 mg of the cross-linked polymer derived from β-cyclodextrin (<125 micron particle size). Water (9.0 mL) was then combined with this portion over the course of 9 minutes at a rate of 1.0 mL/minute. The mixture was then stirred at room temperature for 30 minutes.

One portion of the stock solution was transferred to a vial containing a stir bar and 300 mg of the cross-linked polymer derived from β-cyclodextrin (178-400 micron particle size). An aqueous solution of citric acid (1.0 M, 9.0 mL) was then combined with this portion over the course of 9 minutes at a rate of 1.0 mL/minute to produce a reaction mixture. The reaction mixture was then stirred at room temperature for 30 minutes.

A chromatography column was packed with 1.5 g of the coarsely-ground cyclodextrin polymer (178-400 micron particle size) to height of 3.5 cm with diameter 1 cm. The chromatography medium was flushed with 20 mL ethanol followed by 20 mL water. The first reaction mixture was poured onto the chromatography medium, rinsing the vial with 30 mL water. The liquid was entirely forced through the media using a gentle application of compressed gas. A 10 mL portion of ethanol was added to the top of the column, and the liquid entirely forced through the media in the same manner and collected in a separate receptacle. Using additional portions of ethanol this process was repeated a total of four times.

The CBD content of each fraction was determined and the eluents combined to a total of 70 mL ethanol. The CBD content of combined ethanol fractions was determined. The second reaction mixture was subjected to the same chromatography protocol.

FIG. 96 shows, in addition to the data of Example 37, the total CBD recovered resulting from Example 38. These results demonstrate that a chelating agent can be utilized in the second solvent in addition to water was demonstrated to yield greater recovery of the lipophilic target compound relative to water without a chelating agent and was visually observed to be less colored following the above chromatographic fractionation.

These results imply that solvents distinct from ethanol, specifically ionic liquid 1-butyl-3-methylimidazolium tetrafluoroborate, can be successfully used in conjunction with our capturing device and protocol to recover lipophilic compounds.

### Example 39

Sixty milligrams of CBD were dissolved in 9.0 mL of ethanol according to the protocol set out in Example 17. This stock solution was divided into six portions of 1.5 mL each. One portion of the stock solution was taken to calculate the baseline CBD concentration at t=0 by diluting with 3.5 mL of ethanol.

One portion of the stock solution was transferred to a vial containing a stir bar and 100 mg of the cross-linked polymer derived from beta-cyclodextrin (<125 micron particle size). Three portions of the stock solution were transferred to a vial containing a stir bar and 300 mg of the cross-linked polymer.

Water (3.5 mL) was then combined with the first vial over the course of 7 minutes at a rate of 0.5 mL/minute. Water (10.5 mL) was added to the second vial over the course of 10.5 minutes at a rate of 1.0 mL/minute.

Each vial was transferred to a rotary evaporator and the organic component was removed under reduced pressure. This process was performed slowly over the course of 30 minutes until all volatile organics had been removed a small quantity of aqueous material was observed to be distilling.

The reaction mixtures were then filtered to retrieve the cross-linked polymers. The cross-linked polymer retrieved after filtration of the first vial was suspended in 5 mL ethanol. An aliquot was taken to determine released CBD concentration t=rel.

The cross-linked polymer retrieved after filtration of the second vial was poured onto a chromatography column containing 300 mg cross-linked polymer (<125 micron particle size) that was previously packed using water and dried with compressed air. The column was flushed with water (20 mL) and pumped dry with compressed air. The column was flushed with DMSO (15 mL) to recover the CBD and an aliquot was taken to determine released CBD concentration t=col.

Sixty milligrams of CBD were dissolved in 9.0 mL of acetonitrile according to the protocol set out in Example 17. This stock solution was divided into six portions of 1.5 mL each. One portion of the stock solution was taken to calculate the baseline CBD concentration at t=0 by diluting with 3.5 mL of acetonitrile.

One portion of the stock solution was transferred to a vial containing a stir bar and 100 mg of the cross-linked polymer derived from beta-cyclodextrin (<125 micron particle size). Three portions of the stock solution were transferred to a vial containing a stir bar and 300 mg of the cross-linked polymer.

Water (3.5 mL) was then combined with the first vial over the course of 7 minutes at a rate of 0.5 mL/minute. Water (10.5 mL) was added to the second vial over the course of 10.5 minutes at a rate of 1.0 mL/minute.

Each vial was transferred to a rotary evaporator and the organic component was removed under reduced pressure. This process was performed slowly over the course of 30 minutes until all volatile organics had been removed a small quantity of aqueous material was observed to be distilling.

The reaction mixtures were then filtered to retrieve the cross-linked polymers. The cross-linked polymer retrieved after filtration of the first vial was suspended in 5 mL ethanol. An aliquot was taken to determine released CBD concentration t=rel.

The cross-linked polymer retrieved after filtration of the second vial was poured onto a chromatography column containing 300 mg cross-linked polymer (<125 micron particle size) that was previously packed using water and dried with compressed air. The column was flushed with water (20 mL) and pumped dry with compressed air. The column was flushed with ethanol (50 mL) to recover the CBD. The solution of CBD was subsequently concentrated to a total volume of 15 mL and an aliquot was taken to determine released CBD concentration t=col.

The process was repeated using dichloromethane and also hexane as the initial solvent.

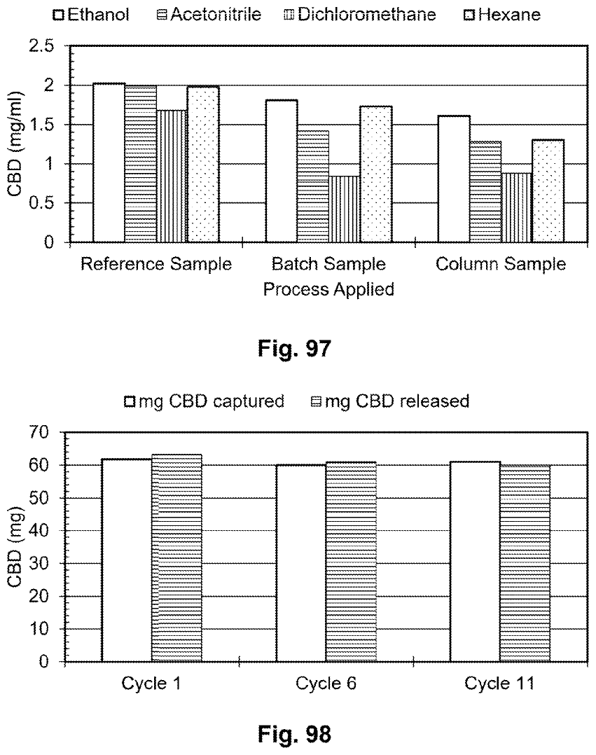

FIG. 97 shows the CBD concentration resulting from Example 39. These results demonstrate that recovery of a lipophilic target substance can be achieved using the cross-linked polymer by gradual evaporation of a lipophilic solvent from an aqueous mixture. By contrast with the ‘standard’ batch protocol, this technique has been employed using both water-miscible and water-immiscible solvents. In addition, these findings demonstrate that lipophilic compounds captured following this technique can be eluted from a chromatography column following dry-loading of the polymer after the capture phase. Specifically, elution is not observed when flushing with a hydrophilic media but is eluted when flushing with more lipophilic solvents.

### Example 40

Eighty-six milligrams of CBD were dissolved in 10 mL of ethanol to produce a stock solution according to the protocol set out in Example 17. One 1.5 mL portion of the stock solution was taken to calculate the baseline CBD concentration at t=0 by diluting with 3.5 mL of ethanol.

One 7.5 ml portion stock solution above was transferred a glass reactor vessel called a peptide synthesis vessel, with an internal separating wall of sintered glass and a closed tap below, containing a stir bar and 100 mg of the cross-linked polymer, and held at a 45 degree angle. Water (17.5 mL) was then combined with this portion over the course of 17.5 minutes at a rate of 1 mL/minute. The mixture was then stirred at room temperature for 30 minutes.

The reaction mixture was then filtered at t=30 to retrieve the cross-linked polymer, by attaching the reaction vessel to Büchner flask under vacuum, and opening the rector tap. An aliquot was taken from the filtrate to calculate the CBD concentration at t=30. The reactor vessel tap was closed and The cross-linked polymer was resuspended in 5 mL DMSO and stirred for 30 minutes before filtering in the same manner. An aliquot was then taken from the filtrate to determine released CBD concentration t=rel.

This process of capture and release was then repeated 4 time without taking aliquots, and then a fifth time while taking aliquots. This set of five capture-release cycles was repeated twice, a total eleven capture-release cycles, including the initial cycle. Aliquots of capture and release after cycles 1, 6 and 11 showed the cross-linked polymer continued to capture and release CBD after uses.

FIG. 98 shows the total CBD captured and released resulting from Example 40. These results demonstrate that that capture of insoluble polysaccharides can be used repeatedly after regeneration to recover lipophilic compounds in a closed system.

### Example 41

Eighty-six milligrams of CBD were dissolved in 10 mL of ethanol to produce a stock solution according to the protocol set out in Example 17. One 1.5 mL portion of the stock solution was taken to calculate the baseline CBD concentration at t=0 by diluting with 3.5 mL of ethanol.

One 7.5 ml portion stock solution above was transferred a 50 mL round bottom flask, containing a stir bar and 100 mg of the cross-linked polymer. Water (17.5 mL) was then combined with this portion over the course of 17.5 minutes at a rate of 1 mL/minute. The mixture was then stirred at room temperature for 30 minutes.

An aliquot was taken and filtered through a glass pipette with cotton wool to calculate the CBD concentration at t=30. The cross-linked polymer was then filtered from the reaction mixture, washed with water, resuspended in 25 mL DMSO and stirred for 30 minutes before taking an aliquot and filtering in the same manner.

This process of capture and release was then repeated 4 times without taking aliquots, and then a fifth time while taking aliquots. This set of five capture-release cycles was repeated twice, a total eleven capture-release cycles, including the initial cycle. Aliquots of capture and release after cycles 1, 6 and 11 showed the cross-linked polymer continued to capture and release CBD after uses.

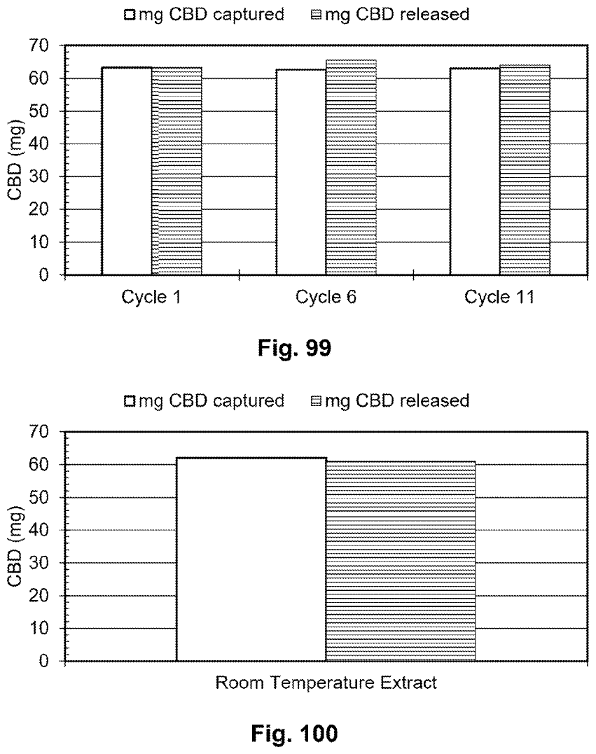

FIG. 99 shows the total CBD captured and released resulting from Example 41.

### Example 42

Eighty-six milligrams of CBD were dissolved in 10 mL of ethanol to produce a stock solution according to the protocol set out in Example 17. One 1.5 mL portion of the stock solution was taken to calculate the baseline CBD concentration at t=0 by diluting with 3.5 mL of ethanol.

One 7.5 ml portion stock solution above was transferred a glass reactor vessel called a peptide synthesis vessel, with an internal separating wall of sintered glass and a closed tap below, containing a stir bar and 100 mg of the cross-linked polymer, and held at a 45 degree angle. Water (17.5 mL) was then combined with this portion over the course of 17.5 minutes at a rate of 1 mL/minute. The mixture was then stirred at room temperature for 30 minutes.

The reaction mixture was then filtered at t=30 to retrieve the cross-linked polymer, by attaching the reaction vessel to Buchner flask under vacuum, and opening the rector tap. An aliquot was taken from the filtrate to calculate the CBD concentration at t=30. The reactor vessel tap was closed and the cross-linked polymer was resuspended in 25 mL DMSO and stirred for 30 minutes before filtering in the same manner. An aliquot was then taken from the filtrate to determine released CBD concentration t=rel.

FIG. 100 shows the total CBD captured and released resulting from Example 42.

### Example 43

Thirty milligrams of CBD were dissolved in 4.5 mL of glucose syrup and stirring until dissolution occurred. This stock solution was divided into 3 portions of 1.5 mL. One portion of the stock solution was taken to calculate the baseline CBD concentration at t=0 by diluting with 3.5 mL of ethanol.

One portion of the stock solution was transferred to vial containing a stir bar and 100 mg of the cross-linked polymer. Water (3.5 mL) was added over the course of 7 minutes at a rate of 0.5 mL/minute. The mixtures was then stirred at 30 minutes with continued heating.

An aliquot was taken and filtered using a syringe filter then used to calculate the CBD concentration at t=30. The reaction mixture was then filtered to retrieve the cross-linked polymer. The cross-linked polymer retrieved after filtration each suspended in 5 mL ethanol. An aliquot was taken to determine released CBD concentration at release.

FIG. 95, in addition to Example 36, shows the mass of CBD resulting from Example 43. This data demonstrate that deep eutectic solvents including a commercial sugar syrup can be employed as the first solvent.

### Example 44

Two hundred and fifty milligrams samples of cross-linked polymer were suspended in a series of buffer solutions and strong acids and bases: pH0 1M HCl, pH1 0.1M HCl/KCl buffer, pH3 0.1M Glycine/HCl buffer, pH4 0.1M citrate buffer, pH5 0.1M acetate buffer, pH7 0.1M phosphate buffer, pH9 0.1M Glycine/NaOH buffer, pH10 0.1M carbonate/bicarbonate buffer, pH13 0.1M NaOH/NaCl buffer, pH14 1M NaOH.

In each case, the polymer was stirred in a vial containing 25 mL of buffer solution for seven days. Samples were then filtered, washed with water and dried, and then analyzed by Fourier transform infrared spectroscopy for structural or chemical changes. No substantial differences were found between the FTIR spectra of the exposed polymers and that of the untreated polymer across the range of pHs and concentrations investigated.

A standard capture and release protocol as demonstrated in Claim 17 was performed on the exposed sampled, which performed to the same standard as the untreated polymer, such as shown in FIG. 85. These results imply that a capturing device can be used to recover lipophilic compounds following exposure to acidic and basic conditions.

### Example 45

250 mg cross-linked polymer was heated in an oven at 120 degrees Celsius for 24 hours. The sample was cooled and analyzed by FTIR spectroscopy for structural or chemical changes. No substantial differences were found between the FTIR spectra of the heated polymer and that of the unheated polymer.

A standard capture and release protocol as demonstrated in Claim 17 was performed on the heated sample, which performed to the same standard as the unheated polymer, such as shown in FIG. 85.

### Example 46

Dried cannabis hemp plant material (2.02 g, 8.5% moisture content; 4.49% CBDA; 0.26% CBD; 0.19% THCA; <0.02% THC) was heated to 110° C. for 40 minutes in a convection oven. The recovered plant material (1.81 g) was transferred to a centrifuge tube containing activated charcoal (200 mg) and a stir bar. Ethanol (30 mL) was added and the mixture stirred vigorously for 3 hours at room temperature. The mixture was centrifuged for 30 minutes at 350 rpm and the amber colored liquid decanted by pipette transfer to a separate container.

From the above stock solution, 6.0 mL was transferred to a vial containing 200 mg of the insoluble polysaccharide and a stir bar. Brine (1.0 M, 14.0 mL) was then combined with this portion over the course of 28 minutes at a rate of 0.5 mL/minute. The mixture was then stirred at room temperature for 30 minutes.

The reaction mixture was then filtered to retrieve the cross-linked polymer. The cross-linked polymer retrieved after filtration was suspended in 6 mL ethanol. An aliquot was taken to determine released phytocannabinoid concentration and composition with comparison to the phytocannabinoid composition of the ethanolic cannabis extract.

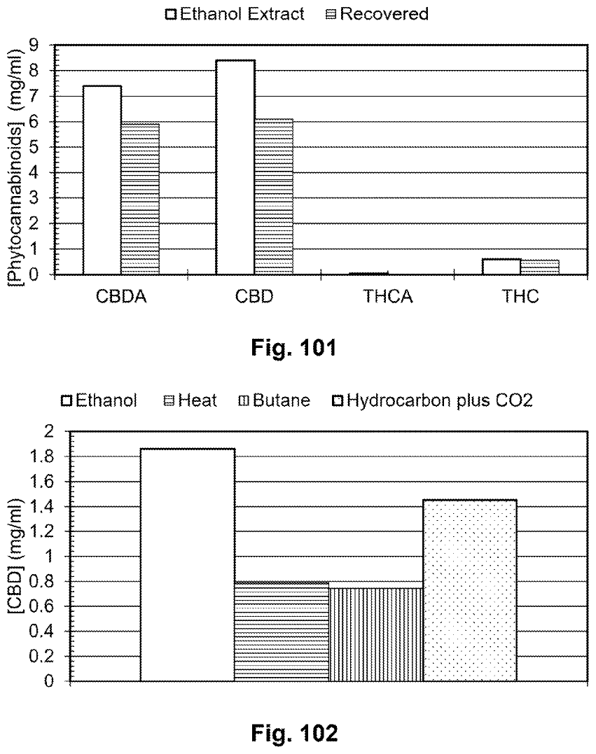

FIG. 101 shows recovery of CBDA, CBD, THCA and THC of Example 46. This data demonstrates that partially decarboxylated cannabis hemp can be recovered using insoluble polysaccharides. This data also demonstrates that CBD and THC can be captured using insoluble polysaccharides and that some selectivity of capture is observed compared with the respective phytocannabinoid acid forms.

### Example 47

130 milligrams of CBD were dissolved in 19.5 mL of ethanol according to the standard protocol. Two portions of 7.5 mL of the stock solution were transferred to two vials each containing 0.5 g of the insoluble polysaccharide polymer and a stir bar.

To each portion, water (17.5 mL) was added over the course of 17 minutes at a rate of 0.5 mL/minute. The mixtures were then stirred at room temperature for 30 minutes. Each reaction mixture was then filtered to retrieve the cross-linked polymers. Each polymer was transferred to a pipette plugged with cotton wool and purged of residual water using a flow of argon for 1 minute.

To the first pipette, butane gas was passed through in a constant stream that was maintained for 10 minutes. The butane having passed through the polymer was collected using a round bottomed flask and spontaneously evaporated under atmospheric pressure to provide the recovered CBD. The mass of the collected CBD was measured (18.6 mg) and the sample dissolved in ethanol (25.0 mL) to verify CBD quantity by HPLC.

To the second pipette, mixture of liquidized gases containing various linear, branched, cyclic, and aromatic hydrocarbons as well as carbon dioxide was passed through in a constant stream that was maintained for 10 minutes. The gases having passed through the polymer were collected using a round bottomed flask and evaporated rapidly under atmospheric pressure to provide the recovered CBD. The mass of the collected CBD was measured (36.3 mg) and the sample dissolved in 25.0 mL ethanol to verify CBD quantity by HPLC.

One 1.5 mL portion of the stock solution was subjected to the standard protocol for slurry batch capture and release using ethanol (5.0 mL) as the releasing solvent. The determined CBD concentration used as a reference comparison.

FIG. 102 shows concentrations of CBD recovered using solvent driven or gas-driven CBD release in Example 47. This data demonstrates that liquidized gases with hydrophobic properties can be employed to promote the release of lipophilic compounds bound to the insoluble polysaccharide. Furthermore, the rapid evaporation of the liquidized can be allow isolation of the lipophilic compound from the polymeric polysaccharide in a solventless form.

### Example 48

130 milligrams of CBD were dissolved in 19.5 mL of ethanol according to the standard protocol. 15 mL of the stock solution was transferred to a vial containing 1.0 g of the insoluble polysaccharide polymer and a stir bar.

Water (45.5 mL) was combined with this portion over the course of 45 minutes at a rate of 1.0 mL/minute. The mixture was then stirred at room temperature for 30 minutes. The reaction mixture was then filtered to retrieve the cross-linked polymer.

The polymer was transferred to a round bottomed flask fitted with a short-path distillation receiving bulb. The system was evacuated under reduced pressure and rotation was initiated. Conventional distillation treatment was applied, whereby the flask containing the polymer was heated and the receiving flask cooled until condensate was observed in the receiving flask.

The mass of the collected CBD was measured (39.4 mg) and the sample dissolved in 50.0 mL ethanol to verify CBD quantity by HPLC. One 1.5 mL portion of the stock solution was subjected to the standard protocol for slurry batch capture and release using ethanol (5.0 mL) as the releasing solvent. The determined CBD concentration used as a reference comparison.

FIG. 102 shows concentrate of CBD recovered using solvent driven or heat-driven CBD release in Example 48. This data also demonstrates that heat can be used to promote the release of lipophilic compounds bound to the insoluble polysaccharide polymer. Furthermore, a conventional short path distillation setup can be employed to isolate the lipophilic compound from the polymeric polysaccharide in a solventless form.

### Example 49

FTIR and capture-release data demonstrate that recovery of lipophilic target compounds after exposure of the insoluble polysaccharide to temperatures up to 120° C. performed to the same standard as the unheated polymer, such as shown in FIG. 85.

### Example 50

FIG. 79 shows the amount in milligrams of CBD captured and released in Example 20. Samples of the captured lipophilic target compounds were resolved on HPLC and measured using UV absorption at 254 nm.

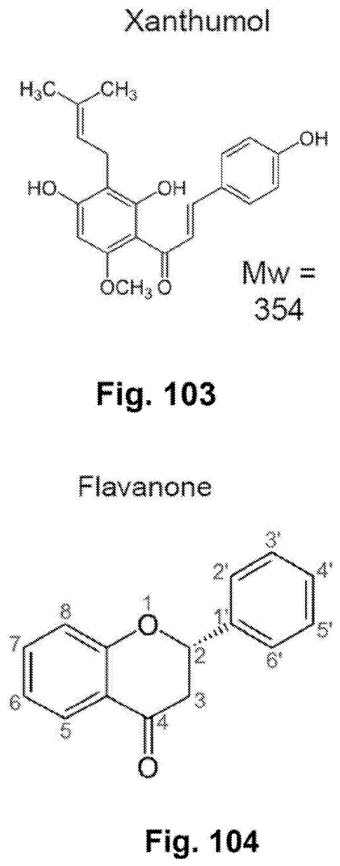

FIGS. 103 and 104 show the chemical structures of xanthumol and flavanone, respectively. Xanthumol and flavanone are structural isomers and have the same molecular weight.

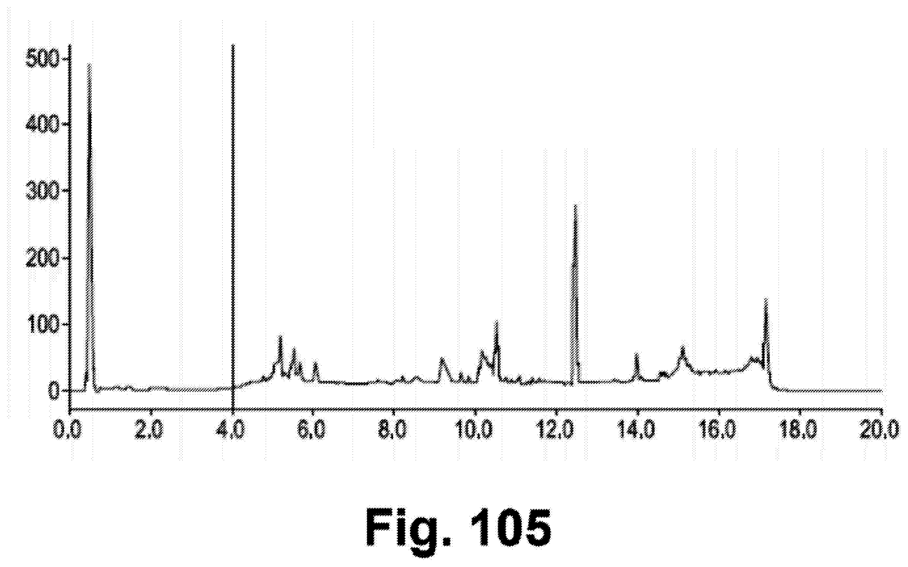

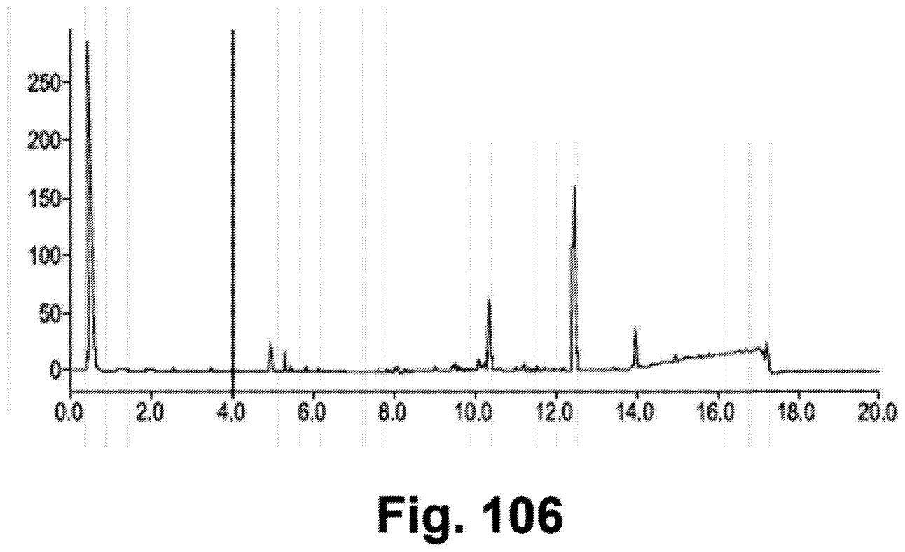

FIGS. 105 and 106 are time-course UV absorption graphs of the reaction mixture before the addition of the cross-linked polymer, and after filtration and flushing of the cross-linked polymer, respectively. The time-course UV spectra show resolution of compounds by HPLC.

In the UV spectra, CBD was visible prior to capture and release at about 12.6 min (FIG. 105). In addition to CBD, other peaks were lowered in FIG. 106, showing collection of other compounds by the polymer as shown by the presence of a larger xanthumol/flavanone peak at about 10.3 min in FIG. 105 than in FIG. 106.

### Example 51

Two-hundred and fifty milligrams of CBD were dissolved in 37.5 mL of ethanol to produce a stock solution according to the protocol set out in Example 17. The stock solution was divided into 25 portions of 1.5 mL. A first portion of the stock solution was taken to calculate the baseline CBD concentration at t=0 by diluting with 3.5 mL of ethanol.

Twenty-two portions of the stock solution were each transferred into twenty-two respective vials, each containing 100 mg of adsorbent with a different adsorbent in every vial. To each portion, water (3.5 mL) was then combined with this portion over the course of 7 minutes at a rate of 0.5 mL/minute. To one of the portions containing cyclodextrin-HDI, brine (3.5 mL) was added in place of water. The mixture was then stirred at room temperature for 30 minutes.

The adsorbents were then retrieved and dried by filtration. The adsorbents were transferred to separate vials and suspended in 5 mL ethanol. From each portion, an aliquot was taken to taken to determine released CBD concentration t=rel.

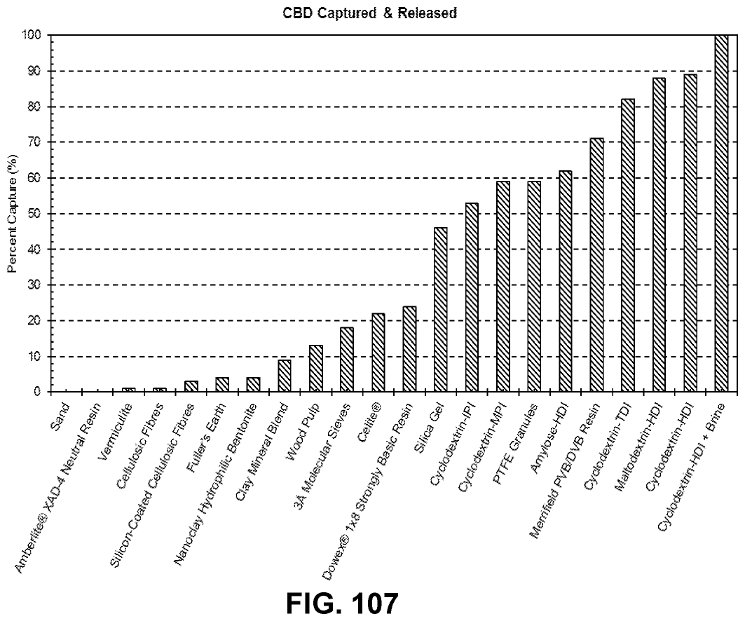

FIG. 107 shows the concentration of released CBD resulting from Example 51 for each of the tested adsorbents. These results demonstrate that one or more cross-linked polymers can be used while physically separated and housed within separate immersion filters. As shown in FIG. 107, adsorbents tested following this procedure and the resulting recovery of CBD may be grouped into a first category that recovered at least 40% of the CBD present in the sample and a second category that recovered less than 25% of the CBD present in the sample. Table 1 shows a breakdown of the same data values displayed in FIG. 107

TABLE 1
Recovery of CBD according to different adsorbents

| Over 40% Recovered | | Less than 25% Recovered | |
|---|---|---|---|
| Cyclodextrin-HDI and brine | 100% | Dowex ® 1 × 8 strongly basic resin | 24% |
| Cyclodextrin-HDI | 89% | Celite ® | 22% |
| Maltodextrin-HDI | 88% | 3 Å molecular sieves | 18% |
| Cyclodextrin-TDI | 82% | Wood pulp | 13% |
| Merrifield PVB/DVB resin | 71% | Clay Mineral Blend | 9% |
| Amylose-HDI | 62% | Nanoclay hydrophilic bentonite | 4% |
| PTFE Granules | 59% | Fuller's earth | 4% |
| Cyclodextrin-MPI | 59% | Silicon-coated cellulosic fibres | 3% |
| Cyclodextrin-IPI | 53% | Cellulosic fibres | 1% |
| Silica gel | 46% | Vermiculite | 1% |
| — | — | Amberlite ® XAD-4 neutral resin | 0% |
| | | Sand | 0% |

Adsorbent Added Prior to Sample

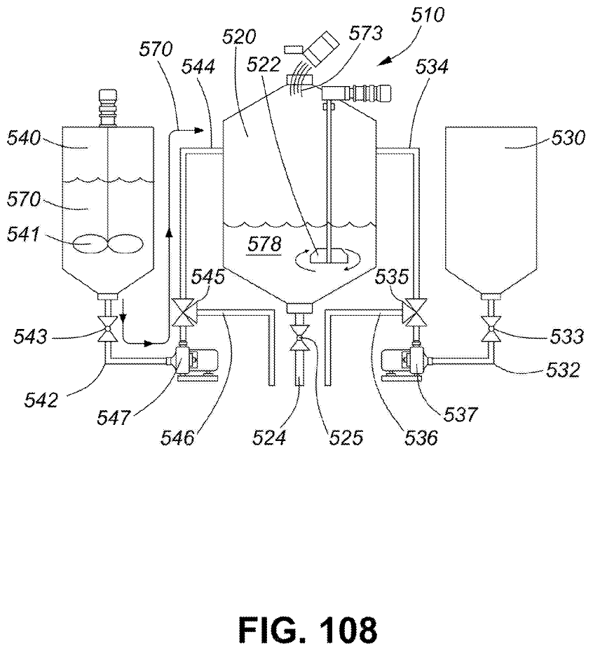

FIGS. 108 to 114 show a system 510 in use to purify a lipophilic target compound from a sample 554 using the insoluble adsorbent 573, the lipophilic solvent 560 and the hydrophilic solvent 570. The lipophilic solvent 560 is stored in and sourced from the lipophilic solvent vessel 530. The hydrophilic solvent 570 is stored in and sourced from the hydrophilic solvent vessel 540. For simplicity of review of FIGS. 108 to 114, the lipophilic solvent 560 and the agitator 531 are shown in the lipophilic solvent vessel 530 only when the lipophilic solvent 560 is being supplied to the slurry tank 520. Similarly, and also for simplicity of review of FIGS. 108 to 114, the hydrophilic solvent 570 and the agitator 541 are shown in the hydrophilic solvent vessel 540 only when the hydrophilic solvent 570 is being supplied to the slurry tank 520. In figures where these solvents are not being supplied to the slurry tank 520, the lipophilic solvent vessel 530 and the hydrophilic solvent vessel 540 are shown without detail. The system 510 facilitates providing the insoluble adsorbent 573 in the hydrophilic solvent 570 rather than in the lipophilic solvent 540.

FIG. 108 shows the insoluble adsorbent 573 being added to the slurry tank 520 and the hydrophilic solvent 570 being added to the slurry tank 520 from the lipophilic solvent vessel 530. The hydrophilic solvent 570 may be added to the slurry vessel 520 via the upstream hydrophilic solvent flow line 542 and the downstream hydrophilic solvent flow line 544, and combined with the insoluble adsorbent 573 to provide a primed slurry 578. The hydrophilic solvent 570 may be provided in a ratio of 75% of the insoluble adsorbent 573 to 25% hydrophilic solvent 570. Depending on the insoluble adsorbent 573 and the hydrophilic solvent 570 that are used, ratios of the insoluble adsorbent 573:hydrophilic solvent 570 may range from 10:90, 9:91, 8:92, 7:93, 6:94, 5:95, 4:96, 3:97, 2:98 or 1:99. Alternatively, either a portion of the insoluble adsorbent 573 or all of the insoluble adsorbent 573 may be added to the slurry vessel 520 after adding the hydrophilic solvent 570 to the slurry vessel 520 (not shown).

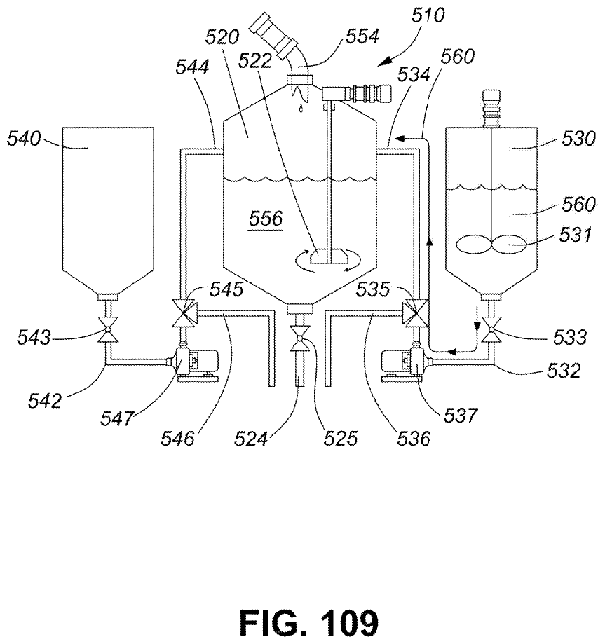

FIG. 109 shows the sample 554 being loaded into the slurry vessel 520 and combined with the primed slurry 578 and the lipophilic solvent 560 being loaded into the slurry vessel 520 to provide the binding slurry 556. The lipophilic solvent 560 may be provided to the slurry vessel 520 from the lipophilic solvent vessel 530 via the upstream lipophilic solvent flow line 532 and the downstream lipophilic solvent flow line 534. The binding slurry 556 may be about 75% lipophilic solvent 560 to 25% hydrophilic solvent 570 where the lipophilic solvent 560 is ethanol and the hydrophilic solvent 570 is water. Depending on the lipophilic solvent 5603 and the hydrophilic solvent 570 that are used, ratios of lipophilic solvent 560:hydrophilic solvent 570 may range from 95:5, 90:10, 85:15, 80:20, 75:25, 70:30, 65:35, 60:40, 55:45, 50:50, 45:55, 40:60, 35:65, 30:70, 25:75, 20:80, 15:85, 10:90 and 5:95. The slurry vessel 520 may be chilled to between 3° C. and room temperature, such as 4° C., when the sample 554 and the lipophilic solvent 560 are added to the slurry vessel 520. In some cases, lower temperatures may also facilitate maintaining a liquid state in a low boiling gaseous solvent, such as butane or other shorter hydrocarbon solvents with boiling points below or close to −1° C. In some cases, lower temperatures may also improve the stability of temperature-sensitive lipophilic target compounds. In some cases, higher temperatures may be applied to decrease solvent viscosity. In some cases, higher temperatures may be used to facilitate in situ decarboxylation of phytocannabinoids, if decarboxylated phytocannabinoids are the target molecule and where decarboxylation was not previous carried out on the sample 554. Temperature may also be modulated to maintain a temperature range at which supercritical fluids have the appropriate physical properties.

The sample 554 includes at least one lipophilic target compound. The sample 554 may include for example an extract or other sample from a biological source (e.g. a plant, animal tissue fungi, yeast, bacteria, or other microorganism), mineral samples (e.g. gold salts, gold complexes, copper salts, copper complexes, etc.), chemical waste samples (e.g. hydrocarbon extraction and processing effluent, mining tailings, etc.). The lipophilic target compound may include any compound that complexes with, binds with or otherwise adheres to the insoluble adsorbent 573. The lipophilic target compound may adhere with the insoluble adsorbent 573 by coordinating within a torus formed by the molecular structure of the insoluble adsorbent 573, or by binding with the insoluble adsorbent 573 outside of the torus.

Where the lipophilic target compound are phytocannabinoids, the sample 554 may be an ethanolic extract of C. sativa flowers or other trichome-bearing biomass, the lipophilic solvent 560 may be ethanol and the hydrophilic solvent 570 may be water, the binding slurry 560 may target a ratio of 30:70 lipophilic solvent 560 to hydrophilic solvent 570 for driving the lipophilic target compounds into the insoluble adsorbent 73 polymer core. Other ratios of lipophilic solvent 60 to hydrophilic solvent 570 for the binding slurry 556 may be selected for other lipophilic solvents 60, hydrophilic solvents 570, samples 554 or target lipophilic compounds. Together, the lipophilic solvent 560 and the hydrophilic solvent 570 in a ratio that pushes the target lipophilic target substance into the insoluble polysaccharide 550 provide a binding solvent 558. The binding solvent 558 may include miscible lipophilic solvent 60 and hydrophilic solvent 570 or immiscible lipophilic solvent 560 and hydrophilic solvent 570 separated into two layers.

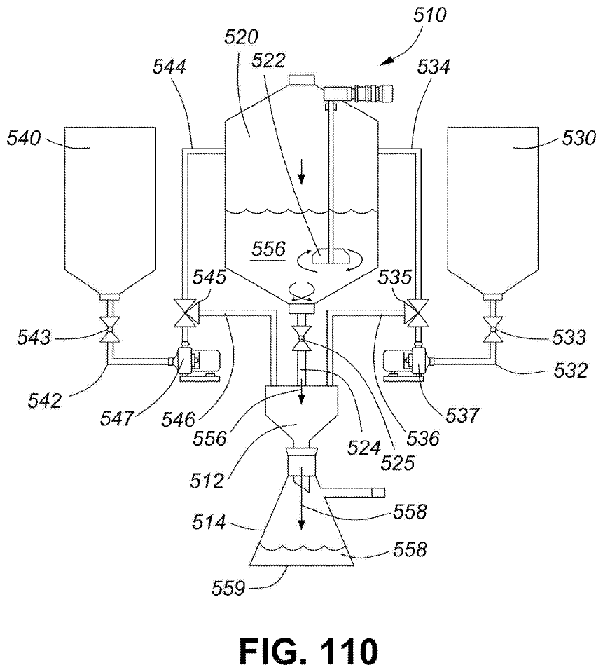

FIG. 110 shows the binding slurry 556 being run through the filter 512 for filtering and retaining the insoluble adsorbent 573 with captured lipophilic target compounds. The binding solvent 558 runs through the filter 512 into the recovery vessel 514. The filter 512 may comprise paramagnetic or other magnetic qualities for magnetically attracting or retaining embodiments of the insoluble adsorbent 573 bound to a magnetic particle or a magnetic nanoparticle on the filter 512.

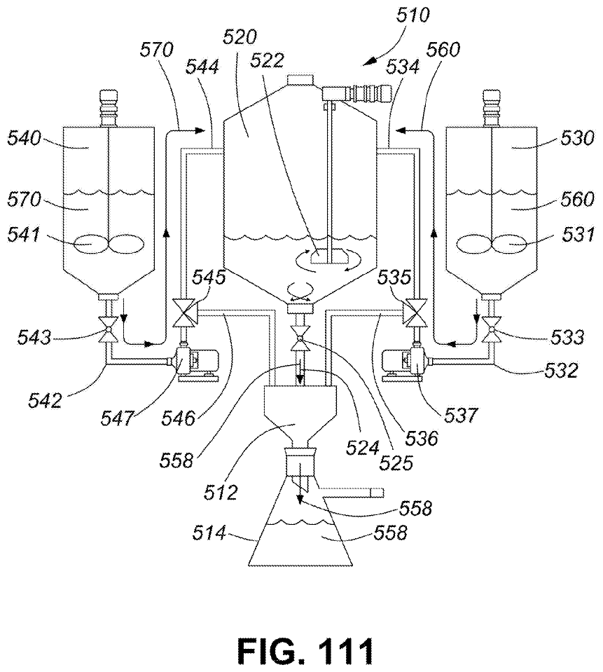

FIG. 111 shows rinsing of the filter 512 with the binding solvent 558 or other ratios of the lipophilic solvent 560 and the hydrophilic solvent 570 to wash the filter 512. Rinsing with the binding solvent 558 may remove some material (e.g. chlorophyll, CBDA, etc.) that water by itself may not remove. This step may also recover some valuable material that binds less strongly than a target hydrophobic material, such as recovery of CBDA when decarboxylated CBD is the primary lipophilic target compound. Such valuable material may be repurified through the system 510. Providing the binding solvent 558 to the filter 512 through the downstream lipophilic solvent flow line 534 and the downstream hydrophilic solvent flow line 544 may rinse out the slurry tank 520. The binding solvent 558 may be provided to the filter 512 by direct application of the lipophilic solvent 560 and the hydrophilic solvent 570 to the filter 512 through the lipophilic solvent rinse flow line 536 and the hydrophilic solvent rinse flow line 546.

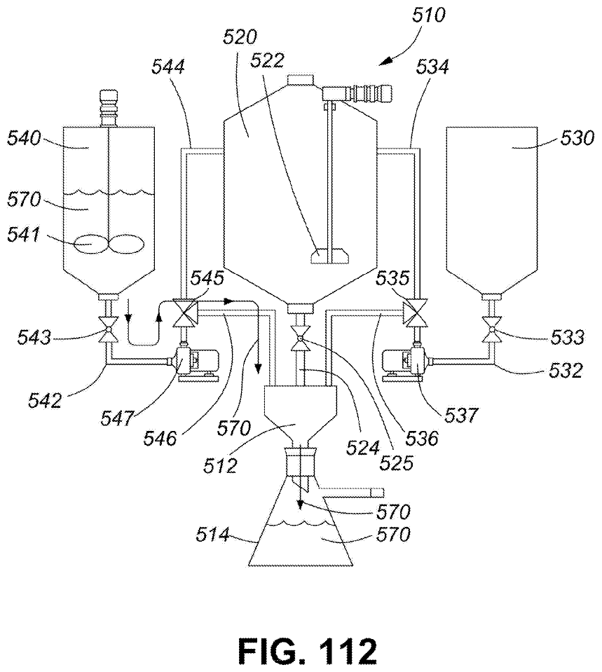

FIG. 112 shows rinsing of the filter 512 with hydrophilic solvent 570 to wash the filter 512 via the upstream hydrophilic solvent flow line 542 and the hydrophilic solvent rinse flow line 546. An amount of hydrophilic solvent 570 used to wash the filter 512 may be about 3 or 4 times the volume of the binding slurry 556 that was passed through the filter 512.

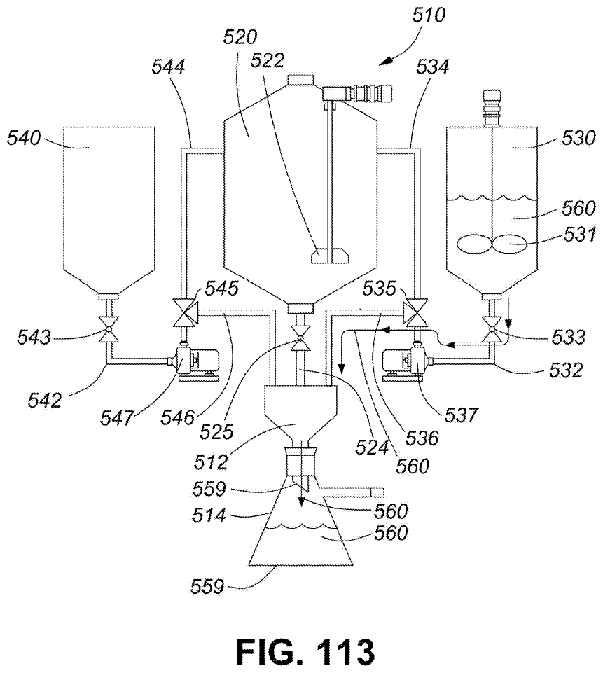

FIG. 113 shows dissolution of the lipophilic target compounds by flowing the lipophilic solvent 560 over the filter 512 to dissociate the lipophilic target compounds from the insoluble adsorbent 573 and solubilize the lipophilic target compounds in the lipophilic solvent 560. A recovered lipophilic target compound 559 is recovered in the lipophilic solvent 560 from the recovery vessel 514 The amount of lipophilic solvent 560 used to recover the recovered lipophilic target compound 559 may be selected to provide the recovered lipophilic target compound 559 at a defined concentration. A lipophilic solvent other than the lipophilic solvent 560 may be used to recover the recovered lipophilic target compound 559.

The insoluble adsorbent 573 may then be regenerated for reuse by washing the insoluble adsorbent 573 with a detergent solution, for example 0.1% Triton X-100 at 37° C. for one minute. Solvents that are able to dissociate any lipophilic compounds from the insoluble adsorbent 573, such as DMSO, may also be applied for regeneration. Exposure to the detergent solution, to solvent or other regeneration may be followed by re-equilibration with 3 to 5 volumes of ethanol.

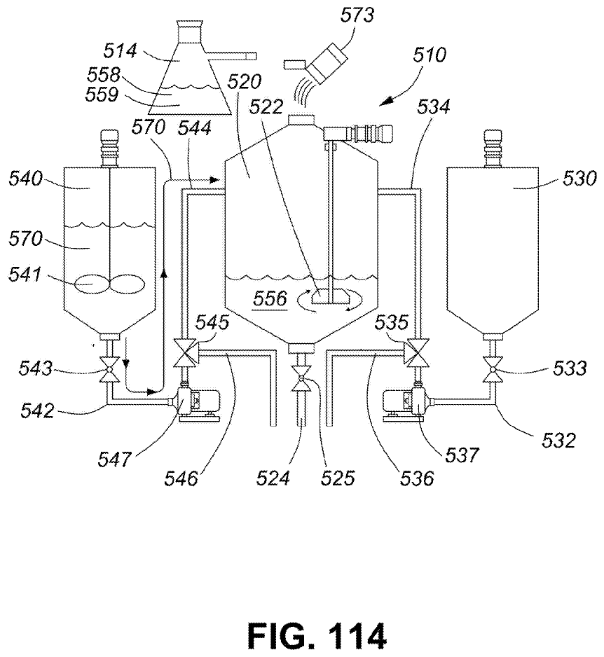

FIG. 114 shows that the contents of the recovery vessel 514 after washing of the ethanol extract may then be loaded into the chilled slurry vessel 520 to repeat the batch slurry, with a second adsorbent. The second adsorbent may be selected based on a structural property or physicochemical property corresponding to a structural property or physiochemical property of a secondary target substance, which may be a hydrophobic or hydrophilic target substance. The structural property or physicochemical property may be surface chemistry, pore size, cavity dimension, stereoelectronic environment and polarity. The second adsorbent may be selected to preferentially adsorb to the secondary target substance.

A similar approach to the application of the system 510 may include use of a Soxhlet apparatus or other distillation apparatus, in which the sample may be obtained from extraction of biomass in the distillation apparatus. A suitable ratio of hydrophilic and lipophilic solvents may be included in the distillation apparatus, such as a 70:30 ratio of water to ethanol may be included in the distillation apparatus and heated to evaporation, leaving a majority of water in the distillation apparatus while hot ethanol evaporates. The hot ethanol may drain through biomass back into the water, mix with the water and bind with the adsorbent that is in the distillation apparatus, whether as insoluble powder, bound to an immersion filter or otherwise localized in the distillation apparatus.

### REFERENCES

Ahmed, S. A.; Ross, S. A.; Slade, D.; Radwan, M. M.; Zulfigar, F.; ElSohly, M. A. J. Nat. Prod. 2008, 71(4), 536-42.
Ameh, S. J.; Obodozie, O. O.; Babalola, P. C.; Gamaniel, K. S. Br. J. Pharmacol. Res. 2011, 1(4), 99-123.
Anderson, R. P.; Zechar, K. Respir. Med. Case Rep. 2019, 26, 171-173; Al-Zouabi, I.: Stogner, J. M.; Miller, B. L.; Lane, E. S. Subst. Abuse Rehabil. 2018, 9, 91-101.
Arslan, M.; Sayin, S.; Yilmaz, M. Tetrahedron: Asymmetry 2013, 24, 982-989.
Azmir, J.; Zaidul, I. S. M.; Rahman, M. M.; Sharif, K. M.; Mohammed, A.; Sahena, F.; Jahurul, M. H. A.; Ghafoor, K.; Norulaini, N. A. N.; Omar, A. K. M. J. Food Eng. 2013, 117, 426-436.
Berman, P.; Futoran, K.; Lewitus, G. M.; Mukha, D.; Benami, M.; Shlomi, T.; Meiri, D. Sci. Rep. 2018, 8(1), 1-15.
Braganca de Carvalho, L.; Carvalho, T. G.; Margriotis, Z. M.; de Castro Ramalho, T.; de Maltos Alves Pinto, L. J. Incl. Phenom. Macrocycl. Chem. 2014, 78, 77-87.
Cai, C.; Yu, W.; Wang, C.; Liu, L.; Li, F.; Tan, Z. J. Mol. Liq. 2019, 287, 110957.
Cordier, C. J.; Morton, D.; Murrison, S.; Nelson, A. S.; O'Leary-Steele, C. Nat. Prod. Rep. 2008, 25, 719-737; (b) Morton, D.; Leach, S.; Cordier, C. J.; Warriner, S.; Nelson, A. Angew. Chem. Int. Ed. 2008, 48, 104-109.
Claude, B.; Morin, P.; Lafosse, M.; Belmont, A.-S.; Haupt, K. Talanta, 2008, 75, 344-350.
Crini, G. Chem. Rev. 2014, 114(21), 10940-10975.
Davison, E. K.; Brimble, M. A. Curr. Opin. Chem. Biol. 2019, 52, 1-8.
Del Valle, E. M. M. Process Biochem. 2004, 39(9), 1033-1046.
Doorenbos, N. J.; Fetterman, P. S.; Quimby, M. W.; Turner, C. E. Ann. N. Y. Acad. Sci. 1971, 191, 3-14; (b) Sexton, M.; Shelton, K.; Haley, P.; West, M. Planta Med. 2018, 84(4), 234-41.
Gaoni, Y.; Mechoulam, R. J. Am. Chem. Soc. 1971, 93, 217-224.
Gilbert, B.; Alves, L. F. Curr. Med. Chem. 2003, 10(1), 13-20.
Grof, C. P. L. Br. J. Clin. Pharmacol. 2018, 84, 2463-2467.
Hazekamp, A.; Verpoorte, R. Eur. J. Pharm. Sci. 2006, 29, 340-47.
Krizek, T.; Bursova, M.; Horsley, R.; Kuchar, M.; Tuma, P.; Cabala, R.; Hlozek, T. J. Cleaner Production 2018, 193, 391-396.
Lewis, M. M.; Yang, Y.; Wasilewski, E.; Clarke, H. A.; Kotra, L. P. ACS Omega 2017, 2, 6091-6103.
Li, G.; Lou, H.-X. Med. Res. Rev. 2017, 38(4), 1255-1294.
Loftsson, T.; Brewster, M. E. J. Pharm. Sci. 1996, 85(10), 1017-1025.
Loomis, W. D.; Battaile, J. Phytochemistry, 1966, 5, 423-438.
Machado, B. A. S.; Pereira, C. G.; Nunes, S. B.; Padilha, F. F.; Umsza-Guez, M. A. Sep. Sci. Technol. 2013, 48(18), 2741-2760.
Mannila, J.; Jarvinen, T.; Jsrvinen, K.; Jarho, P. J. Pharm. Sci. 2007, 96, 312-319.
Morin-Crini, N.; Winterton, P.; Fourmentin, S.; Wilson, L. D.; Fenyvesi, E.; Crini, G. Progress in Polymer Science, 2018, 78, 1-23.
Moulahcene, L.; Skiba, M.; Bounoure, F.; Benamor, M.; Milon, N.; Hallouard, F.; Lahiana-Skiba, M. Int. J. Environ. Res. Public Health 2019, 16, 414.
Ogawa, N.; Takahashi, C.; Yamamoto, H. J. Pharm. Sci. 2015, 104(3), 942-954.
Otta K., Zsadon B., Faragó J., Szejtli J., Tüdös F. (1988) Cyclodextrin—Cellulose Copolymers. In: Huber O., Szejtli J. (eds) Proceedings of the Fourth International Symposium on Cyclodextrins. Advances in Inclusion Science, vol 5. Springer, Dordrecht.
Peng, X.; Duan, M.-H.; Yao, X.-H.; Zhao, C.-J.; Zu, Y.-G.; Fu, Y.-J. Separation and Purification Technol. 2016, 157, 249-257.
Philippova, O.; Barabanova, A.; Molchanov, V.; Khokhlov A. Eur. Pol. J. 2011, 47, 42-559.
Pushpangadan, P.; George, V. Handbook of Herbs and Spices (Second Edition), Volume 1, 2012.
Radosevic, K.; Curko, N.; Srcek, V. G.; Bubalo, M. C.; Tomasevic, M.; Ganic, K. K.; Redovnikovic, I. R. LWT 2016, 73, 45-51.
Rajewski, R. A.; Stella, V. J. J. Pharm. Sci. 1996, 85(11), 1142-1169.
Rates, S. M. K. Toxicon 2001, 39, 603-613.
Rovetto, L. J.; Aieta, N. V. J. Supercrit. Fluids 2017, 129, 16-27.
Ruesgas-Ramon, M.; Figueroa-Espinoza, M. C.; Durand, E. J. Agric. Food Chem. 2017, 65, 3591-3601.
Russo, E. B. Br. J. Pharm. 2011, 163(7), 1344-1364.
Starmans, D. A. J.; Nijhuis, H. H. Trends in Food Science and Technology 1996, 7(6), 191-197.
Still, C. W.; Kahn, M.; Mitra, A. J. Org. Chem. 1978, 14, 2923-2925.
Turner, C. E.; Cheng, P. C.; Lewis, G. S.; Russell, M. H.; Sharma, G. K. Planta Med. 1979, 37(11), 217-225.
Ventura, S. P. M.; e Silva, F. A.; Quental, M. V.; Mondal, D.; Freire, M. G.; Coutinho, J. A. P. Chem. Rev. 2017, 117, 6984-7052.
Yamasaki, H.; Makihata, Y.; Fukunaga, K. J. Chem. Technol. Biotechnol. 2006, 81, 1271-1276.
Yamasaki, H.; Makihata, Y.; Fukunaga, K. J. Chem. Technol. Biotechnol. 2008, 83, 991-997.
Ying, Z.; Shufen, L.; Xiwen, W.; Xing, Z. Chin. J. Chem. Eng. 2007, 15(6), 872-876.
Yue, D.; Yang, L.; Liu, S.; Li, J.; Li, W.; Ma, C. Molecules, 2016, 21, 204-217.
Zainal-Abidin, M. H.; Hayyan, M.; Hayyan, A.; Jayakumar, N. S. Anal, Chim. Acta. 2017, 979, 1-23.
Zulfigar, F.; Ross, S. A.; Slade, D.; Ahmed, S. A.; Radwan, M. M.; Ali, Z.; Khan, I. A.; ElSohly, M. A. Tetrahedron Lett. 2012, 53(28), 3560-3562.

In the preceding description, for purposes of explanation, numerous details are set forth in order to provide a thorough understanding of the embodiments. However, it will be apparent to one skilled in the art that these specific details are not required.

The above-described embodiments are intended to be examples only. Alterations, modifications and variations can be effected to the particular embodiments by those of skill in the art. The scope of the claims should not be limited by the particular embodiments set forth herein, but should be construed in a manner consistent with the specification as a whole.

1-171. (canceled)

172. A method of selectively recovering a phytocannabinoid, the method comprising:
providing a solution comprising the phytocannabinoid in a lipophilic solvent;
combining an adsorbent with the solution;
combining an adsorption solvent with the solution, the adsorption solvent being less hydrophobic than the lipophilic solvent to facilitate binding of the adsorbent with the phytocannabinoid; and
isolating the adsorbent from the solution;
wherein the adsorbent is insoluble in the solution; and
the adsorbent is selected from the group consisting of amylose, maltodextrin, and acetylated cellulose.

173. The method of claim 172 wherein providing the solution comprises combining bulk plant material with the lipophilic solvent and separating the bulk plant material from the lipophilic solvent.

174. The method of claim 173 wherein the bulk plant material comprises material from Cannabis sativa.

175. The method of claim 172 wherein the lipophilic solvent comprises an organic solvent.

176. The method of claim 175 wherein the organic solvent is selected from the group consisting of acetone, acetonitrile, tetrahydrofuran, glycerol, DMSO, dichloromethane and chloroform.

177. The method of claim 175 wherein the organic solvent comprises an alcohol.

178. The method of claim 177 wherein the alcohol is selected from the group consisting of methanol, ethanol, n-propyl alcohol and isopropyl alcohol.

179. The method of claim 175 wherein the organic solvent comprises a hydrocarbon.

180. The method of claim 179 wherein the hydrocarbon is selected from the group consisting of n-hexane, butane and propane.

181. The method of claim 172 wherein the adsorption solvent comprises water.

182. The method of claim 172 wherein combining the adsorption solvent with the solution comprises evaporating at least a portion of the lipophilic solvent prior to combining the adsorption solvent with the solution.

183. The method of claim 172 wherein the adsorbent comprises amylose or maltodextrin and is cross-linked with a cross-linker selected from the group consisting of hexamethylene diisocyanate, isophorone diisocyanate, 4,4′-methylenebis(phenyl isocyanate) and tolylene-2,4-diisocyanate.

184. The method of claim 172 wherein the adsorbent comprises a powder that is insoluble in the solution.

185. The method of claim 172 wherein the adsorbent comprises a gel matrix that is insoluble in the solution.

186. The method of claim 172 wherein the adsorbent is sequestered within a permeable material.

187. The method of claim 172 wherein the adsorbent is added to the solution before combining the adsorption solvent with the solution.

188. The method of claim 172 wherein the adsorbent is added to the solution after combining the adsorption solvent with the solution.

189. The method of claim 172 wherein the phytocannabinoid is ionizable in the solution, and further comprising combining a solute with the solution for competing with the phytocannabinoid for binding on the adsorbent to dissociate the phytocannabinoid from the adsorbent into the solution, for recovering the phytocannabinoid.

190. The method of claim 172 further comprising increasing a temperature of the adsorbent for dissociating the phytocannabinoid from the adsorbent and recovering the phytocannabinoid.

191. The method of claim 172 further comprising combining a dissociation fluid with the adsorbent for dissociating the phytocannabinoid from the adsorbent and recovering the phytocannabinoid.

192. The method of claim 191 wherein the dissociation fluid comprises a fluid selected from the group consisting of methanol, ethanol, n-propyl alcohol and isopropyl alcohol, other alcohols, acetone, acetonitrile, tetrahydrofuran, glycerol, DMSO, dichloromethane, chloroform, other organic solvents, n-hexane, butane, propane, other hydrocarbons, glucose syrup, acetic acid mixed with menthol, other eutectic solvents, 1-butyl-3-methylimidazolium tetrafluoroborate, other ionic liquids, a heated gas, a pressurized gas, subcritical CO₂, other subcritical fluids, supercritical CO₂ or other supercritical fluids.

193. The method of claim 191 wherein the dissociation fluid has a lower volume than the lipophilic solvent for concentrating the phytocannabinoid relative to the concentration of the phytocannabinoid in the solution.

194. The method of claim 191 wherein the dissociation fluid is more hydrophobic than the lipophilic solvent.

195. The method of claim 172 further comprising combining a secondary adsorbent with the solution for binding of the secondary adsorbent with a secondary target substance;
combining a secondary adsorption solvent with the solution to facilitate binding of the secondary adsorbent with the secondary target substance; and
isolating the secondary adsorbent from the solution; wherein
the secondary adsorbent is insoluble in the solution; and
the secondary adsorbent carries a structural property or physicochemical property corresponding to a structural property or physiochemical property of the secondary target substance to preferentially adsorb to the secondary target substance over the phytocannabinoid.

196. The method of claim 195 wherein the structural property or physicochemical property is selected from the group consisting of surface chemistry, pore size, cavity dimension, stereoelectronic environment and polarity.

197. The method of claim 195 wherein the secondary target substance is a secondary lipophilic target substance, the secondary adsorption solvent is more hydrophilic than the solution and the secondary adsorbent is selected from the group consisting of a cyclic polysaccharide, amylose, maltodextrin, silica gel, polytetrafluoroethylene granules, chloromethylpolystyrene-divinylbenzene resin, polyvinyl butyral resin, acetylated cellulose, branched polysaccharides, linear polysaccharides, oligosaccharides, peptides, proteins, polymerized adducts of amino acids, polyphenolic scaffolds, polymeric isoprenes, fatty acid polyesters, alumina, zeolitic molecular sieves and silicon dioxide.

198. The method of claim 197 wherein the secondary adsorbent comprises a secondary cyclodextrin is selected from the group consisting of α-cyclodextrin, β-cyclodextrin and γ-cyclodextrin.

199. The method of claim 197 wherein the secondary adsorbent comprises amylose or maltodextrin.

200. The method of claim 195 wherein the secondary target substance is ionizable in the solution, and further comprising combining a solute with the solution for competing with the secondary target substance for binding on the secondary adsorbent to dissociate the secondary target substance from the secondary adsorbent into the solution, for recovering the secondary target substance.

201. The method of claim 195 further comprising combining a secondary dissociation fluid with the secondary adsorbent for dissociating the secondary target substance from the secondary adsorbent and recovering the secondary target substance.
